(19) United States
(12) Patent Application Publication
(10) Pub. No.: US 2007/0191334 A1
(43) Pub. Date: Aug. 16, 2007

(54) Tricyclic imidazopyridines

(75) Inventors: Andreas Palmer, Singen (DE); Wilm Buhr, Konstanz (DE)

Correspondence Address: NATH & ASSOCIATES PLLC, 112 South West Street, Alexandria, VA, 22314, US

(73) Assignee: ALTANA PHARMA AG, KONSTANZ DE (DE)

(21) Appl. No.: 10/591,957

(22) PCT Filed: Mar. 16, 2005
(86) PCT No.: PCT/EP05/51211

§ 371 Date: Sep. 8, 2006

(30) Foreign Application Priority Data
Mar. 17, 2004 (EP) 04101092.7
Dec. 14, 2004 (EP) 04106577.2

(51) Int. Cl. A61K 31/5377; A61K 31/496; A61K 31/4745; C07D 491/14

(52) U.S. Cl. 514/210.21; 514/233.8; 514/253.03; 514/292; 544/127; 544/361; 546/81

(57) ABSTRACT

The invention provides compounds of the formula I 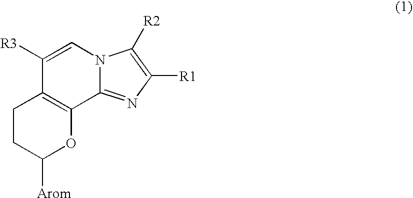 in which the substitutents and symbols are as defined in the description. The compounds inhibit the secretion of gastric acid.

### TECHNICAL FIELD

The invention relates to novel compounds, which are used in the pharmaceutical industry as active compounds for preparing medicaments.

### PRIOR ART

U.S. Pat. No. 4,468,400 describes tricyclic imidazo[1,2-a]pyridines having different ring systems fused to the imidazopyridine skeleton, which compounds are said to be suitable for treating peptide ulcer disorders. The International Patent Applications WO 95/27714, WO 98/42707, WO 98/54188, WO 00/17200, WO 00/26217, WO 00/50037, WO 00/63211, WO 01/72756, WO 01/72754, WO 01/72755, WO 01/72757, WO 02/34749, WO 03/014120, WO 03/016310, WO 03/014123, WO 03/068774 and WO 03/091253 disclose tricyclic imidazopyridine derivatives having a very specific substitution pattern, which compounds are likewise said to be suitable for treating gastrointestinal disorders.

J. J. Kaminski et al., J. Med. Chem. 1985, 28, 876-892, describe the cytoprotective properties of certain imidazopyridines.

### DESCRIPTION OF THE INVENTION

The invention provides compounds of the formula 1 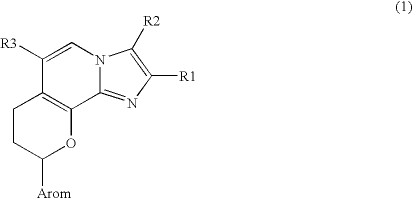
where
R1 is hydrogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl or hydroxy-1-4C-alkyl,
R2 is hydrogen, 1-4C-alkyl, 3-7C-cycloalkyl, 3-7C-cycloalkyl-1-4C-alkyl, 1-4C-alkoxycarbonyl, hydroxy-1-4C-alkyl, hydroxy-3-4-C-alkenyl, hydroxy-3-4C-alkinyl, halogen, 2-4C-alkenyl, 2-4C-alkynyl, fluoro-1-4C-alkyl, cyanomethyl, hydroxy, 1-4C-alkoxy, amino, mono- or di-1-4C-alkylamino, 1-4C-alkylcarbonylamino, 1-4C-alkoxycarbonylamino, 1-4C-alkoxy-1-4C-alkoxycarbonylamino, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR21R22,
   where
   R21 is hydrogen, 1-7C-alkyl, hydroxy-1-4C-alkyl, 1-4C-alkoxy-1-4C-alkyl or 3-7C-cycloalkyl and
   R22 is hydrogen, 1-7C-alkyl, hydroxy-1-4C-alkyl, 1-4C-alkoxy-1-4C-alkyl or 3-7C-cycloalkyl,
   or where
   R21 and R22 together and including the nitrogen atom to which they are attached form a pyrrolidino, piperidino, morpholino, aziridino or azetidino radical,
R3 is 1-4C-alkylcarbonyl, hydroxy-1-4C-alkyl, 1-4C-alkoxy-1-4C-alkyl, 1-4C-alkoxy-1-4C-alkoxy-1-4C-alkyl, 1-4C-alkoxycarbonyl, fluoro-1-4C-alkoxy-1-4C-alkyl, cyano, the radical —CO—NR31R32, the radical —SO₂—NR31R32, the radical —CS—NR31R32, the radical —C═N(OH)—NR31R32 or the group Het
   where
   R31 is hydrogen, amino, 1-7C-alkyl, hydroxy, hydroxy-1-4C-alkyl, 1-4C-alkoxy, 1-4C-alkoxy-1-4C-alkyl, 3-7C-cycloalkyl, 1-4C-alkylsulfonyl, arylsulfonyl, aryl-1-4C-alkylsulfonyl or aryl and
   R32 is hydrogen, 1-7C-alkyl, hydroxy-1-4C-alkyl, 1-4C-alkoxy-1-4C-alkyl or 3-7C-cycloalkyl,
   or where
   R31 and R32 together and including the nitrogen atom to which they are attached form a cyclic residue, substituted by R33, R34 and R35, selected from the group consisting of pyrrolidino, piperidino, piperazino, morpholino, aziridino or azetidino, and
   Het is a heterocyclic residue, substituted by R33, R34 and R35, selected from the group consisting of oxadiazol, dihydrooxazol, dihydroimidazol, oxazol, imidazol, isoxazol, dihydroisoxazol, pyrazol, and tetrazol
   where
   R33 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R34 is hydrogen, 1-4C-alkyl, 1-4C-alkoxy, 1-4C-alkoxycarbonyl, halogen, trifluormethyl or hydroxy,
   R35 is hydrogen, 1-4C-alkyl, 1-4C-alkoxy, 1-4C-alkoxycarbonyl, halogen, trifluormethyl or hydroxy,
Arom is a R4-, R5-, R6- and R7-substituted mono- or bicyclic aromatic radical selected from the group consisting of phenyl, naphthyl, pyrrolyl, pyrazolyl, imidazolyl, 1,2,3-triazolyl, indolyl, benzimidazolyl, furanyl (furyl), benzofuranyl (benzofuryl), thiophenyl (thienyl), benzothiophenyl (benzothienyl), thiazolyl, isoxazolyl, pyridinyl, pyrimidinyl, quinolinyl and isoquinolinyl, where
   R4 is hydrogen, 1-4C-alkyl, hydroxy-1-4C-alkyl, 1-4C-alkoxy, 2-4C-alkenyloxy, 1-4C-alkylcarbonyl, carboxyl, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryloxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R5 is hydrogen, 1-4C-alkyl, 1-4C-alkoxy, 1-4C-alkoxycarbonyl, halogen, trifluoromethyl or hydroxy,
   R6 is hydrogen, 1-4C-alkyl or halogen and
   R7 is hydrogen, 1-4C-alkyl or halogen,
   where
   aryl is phenyl or substituted phenyl having one, two or three identical or different substitutents from the group consisting of 1-4C-alkyl, 1-4C-alkoxy, carboxyl, 1-4C-alkoxycarbonyl, halogen, trifluoromethyl, nitro, trifluoromethoxy, hydroxy and cyano,
with the proviso that,
when
R2 is hydrogen, 1-4C-alkyl, 3-7C-cycloalkyl, 3-7C-cycloalkyl-1-4C-alkyl, 1-4C-alkoxycarbonyl, hydroxy-1-4C-alkyl, halogen, 2-4C-alkenyl, 2-4C-alkynyl, fluoro-1-4C-alkyl or cyanomethyl,
then
R3 is 1-4C-alkylcarbonyl, cyano, the radical —CO—NR31R32, the radical —SO₂—NR31R32, the radical —CS—NR31R32, the radical C═N(OH)—NR31R32 or the group Het
   where for the radical —CO—NR31R32
   R31 is amino, hydroxy, 1-4C-alkoxy, 3-7C-cycloalkyl, 1-4C-alkylsulfonyl, arylsulfonyl, aryl-1-4C-alkylsulfonyl, aryl and
   R32 is hydrogen, 1-7C-alkyl, hydroxy-1-4C-alkyl, 1-4C-alkoxy-1-4C-alkyl or 3-7C-cycloalkyl, and for the radicals —SO₂—NR31R32, CS—NR31R32, and C═N(OH)—NR31R32
   R31 is hydrogen, amino, 1-7C-alkyl, hydroxy, hydroxy-1-4C-alkyl, 1-4C-alkoxy, 1-4C-alkoxy-1-4C-alkyl, 3-7C-cycloalkyl, 1-4C-alkylsulfonyl, arylsulfonyl, aryl-1-4C-alkylsulfonyl or aryl and
   R32 is hydrogen, 1-7C-alkyl, hydroxy-1-4C-alkyl, 1-4C-alkoxy-1-4C-alkyl or 3-7C-cycloalkyl,
   or where
   R31 and R32 together and including the nitrogen atom to which they are attached form a cyclic residue, substituted by R33, R34 and R35, selected from the group consisting of pyrrolidino, piperidino, piperazino, morpholino, aziridino or azetidino where in the case of pyrrolidino, piperidino, or morpholino, at least one of the substitutents R33, R34, or R35 has to be different from hydrogen, and
   Het is a heterocyclic residue, substituted by R33, R34 and R35, selected from the group consisting of oxadiazol, dihydrooxazol, dihydroimidazol, oxazol, imidazol, isoxazol, dihydroisoxazol, pyrazol, and tetrazol
   where
   R33 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R34 is hydrogen, 1-4C-alkyl, 1-4C-alkoxy, 1-4C-alkoxycarbonyl, halogen, trifluormethyl or hydroxy,
   R35 is hydrogen, 1-4C-alkyl, 1-4C-alkoxy, 1-4C-alkoxycarbonyl, halogen, trifluormethyl or hydroxy,
      and their salts.

1-4C-Alkyl denotes straight-chain or branched alkyl radicals having 1 to 4 carbon atoms. Examples which may be mentioned are the butyl, isobutyl, sec-butyl, tert-butyl, propyl, isopropyl, ethyl and methyl radicals.

3-7C-Cycloalkyl denotes cyclopropyl, cyclobutyl, cyclopentyl, cyclohexyl and cycloheptyl, among which cyclopropyl, cyclobutyl and cyclopentyl are preferred.

3-7C-Cycloalkyl-1-4C-alkyl denotes one of the abovementioned 1-4C-alkyl radicals which is substituted by one of the abovementioned 3-7C-cycloalkyl radicals. Examples which may be mentioned are the cyclopropylmethyl, the cyclohexylmethyl and the cyclohexylethyl radicals.

1-4C-Alkoxy denotes radicals which, in addition to the oxygen atom, contain a straight-chain or branched alkyl radical having 1 to 4 carbon atoms. Examples which may be mentioned are the butoxy, iso butoxy, sec-butoxy, tert-butoxy, propoxy, isopropoxy and preferably the ethoxy and methoxy radicals.

1-4C-Alkoxy-1-4C-alkyl denotes one of the abovementioned 1-4C-alkyl radicals which is substituted by one of the abovementioned 1-4C-alkoxy radicals. Examples which may be mentioned are the methoxymethyl, the methoxyethyl and the butoxyethyl radicals.

1-4C-Alkoxycarbonyl (—CO-1-4C-alkoxy) denotes a carbonyl group to which is attached one of the abovementioned 1-4C-alkoxy radicals. Examples which may be mentioned are the methoxycarbonyl (CH₃O—C(O)—) and the ethoxycarbonyl (CH₃CH₂O—C(O)—) radicals.

2-4C-Alkenyl denotes straight-chain or branched alkenyl radicals having 2 to 4 carbon atoms. Examples which may be mentioned are the 2-butenyl, 3-butenyl, 1-propenyl and the 2-propenyl (allyl) radicals.

2-4C-Alkynyl denotes straight-chain or branched alkynyl radicals having 2 to 4 carbon atoms. Examples which may be mentioned are the 2-butynyl, the 3-butynyl and, preferably, the 2-propynyl (propargyl radicals).

Fluoro-1-4C-alkyl denotes one of the abovementioned 1-4C-alkyl radicals which is substituted by one or more fluorine atoms. An example which may be mentioned is the trifluoromethyl radical.

Hydroxy-1-4C-alkyl denotes abovementioned 1-4C-alkyl radicals which are substituted by a hydroxy group. Examples which may be mentioned are the hydroxymethyl, the 2-hydroxyethyl and the 3-hydroxypropyl radicals.

3-4C-Alkenyl denotes straight-chain or branched alkenyl radicals having 3 to 4 carbon atoms. Examples which may be mentioned are the 2-butenyl, 3-butenyl, 1-propenyl and the 2-propenyl (allyl) radicals.

3-4C-Alkynyl denotes straight-chain or branched alkynyl radicals having 3 to 4 carbon atoms. Examples which may be mentioned are the 2-butynyl, the 3-butynyl and, preferably, the 2-propynyl (propargyl radicals).

Hydroxy-3-4-C-alkenyl denotes abovementioned 3-4-C-alkenyl radicals which are substituted by a hydroxy group. Examples which may be mentioned are the 1-hydroxypropenyl or the 1-hydroxy-2-butenyl radical.

Hydroxy-3-4-C-alkinyl denotes abovementioned 3-4C-alkinyl radicals which are substituted by a hydroxy group. Examples which may be mentioned are the 1-hydroxypropinyl or the 1-hydroxy-2-butinyl radical.

For the purpose of the invention, halogen is bromine, chlorine and fluorine.

1-4C-Alkoxy-1-4C-alkoxy denotes one of the abovementioned 1-4C-alkoxy radicals which is substituted by a further 1-4C-alkoxy radical. Examples which may be mentioned are the radicals 2-(methoxy)ethoxy (CH₃—O—CH₂—CH₂—O—) and 2-(ethoxy)ethoxy (CH₃—CH₂—O—CH₂—CH₂—O—).

1-4C-Alkoxy-1-4C-alkoxy-1-4C-alkyl denotes one of the abovementioned 1-4C-alkoxy-1-4C-alkyl radicals which is substituted by one of the abovementioned 1-4C-alkoxy radicals. An example which may be mentioned is the radical 2-(methoxy)ethoxymethyl (CH₃—O—CH₂—CH₂—O—CH₂—).

Fluoro-1-4C-alkoxy-1-4C-alkyl denotes one of the abovementioned 1-4C-alkyl radicals which is substituted by a fluoro-1-4C-alkoxy radical. Here, fluoro-1-4C-alkoxy denotes one of the abovementioned 1-4C-alkoxy radicals which is fully or predominantly substituted by fluorine. Examples of fully or predominantly fluorine-substituted 1-4C-alkoxy which may be mentioned are the 1,1,1,3,3,3-hexafluoro-2-propoxy, the 2-trifluoromethyl-2-propoxy, the 1,1,1-trifluoro-2-propoxy, the perfluoro-tert-butoxy, the 2,2,3,3,4,4,4-heptafluoro-1-butoxy, the 4,4,4-trifluoro-1-butoxy, the 2,2,3,3,3-pentafluoropropoxy, the perfluoroethoxy, the 1,2,2-trifluoroethoxy, in particular the 1,1,2,2-tetrafluoroethoxy, the 2,2,2-trifluoroethoxy, the trifluoromethoxy and preferably the difluoromethoxy radicals.

1-7C-Alkyl denotes straight-chain or branched alkyl radicals having 1 to 7 carbon atoms. Examples which may be mentioned are the heptyl, isoheptyl-(5-methylhexyl), hexyl, isohexyl-(4-methylpentyl), neohexyl-(3,3-dimethylbutyl), pentyl, isopentyl-(3-methylbutyl), neopentyl-(2,2-dimethylpropyl), butyl, isobutyl, sec-butyl, tert-butyl, propyl, isopropyl, ethyl and methyl radicals.

1-4C-Alkylcarbonyl denotes a radical which, in addition to the carbonyl group, contains one of the abovementioned 1-4C-alkyl radicals. An example which may be mentioned is the acetyl radical.

2-4-C-Alkenylcarbonyl denotes a radical which, in addition to the carbonyl group, contains one of the abovementioned 2-4C-alkenyl radicals. An example which may be mentioned is the ethenylcarbonyl or the 2-propenylcarbonyl radical.

2-4-C-Alkinylcarbonyl denotes a radical which, in addition to the carbonyl group, contains one of the abovementioned 2-4C-alkinyl radicals. An example which may be mentioned is the ethinylcarbonyl or the 2-propinylcarbonyl radical.

Carboxy-1-4C-alkyl denotes, for example, the carboxymethyl (—CH₂COOH) or the carboxyethyl (—CH₂CH₂COOH) radical.

1-4C-Alkoxycarbonyl-1-4C-alkyl denotes one of the abovementioned 1-4C-alkyl radicals which is substituted by one of the abovementioned 1-4C-alkoxycarbonyl radicals. An example which may be mentioned is the ethoxycarbonylmethyl (CH₃CH₂OC(O)CH₂—) radical.

Di-1-4C-alkylamino denotes an amino radical which is substituted by two identical or different of the abovementioned 1-4C-alkyl radicals. Examples which may be mentioned are the dimethylamino, the diethylamino and the diisopropylamino radicals.

1-4C-Alkoxycarbonylamino denotes an amino radical which is substituted by one of the abovementioned 1-4C-alkoxycarbonyl radicals. Examples which may be mentioned are the ethoxycarbonylamino and the methoxycarbonylamino radicals.

1-4C-Alkoxy-1-4C-alkoxycarbonyl denotes a carbonyl group to which one of the abovementioned 1-4C-alkoxy-1-4C-alkoxy radicals is attached. Examples which may be mentioned are the 2-(methoxy)ethoxycarbonyl (CH₃—O—CH₂CH₂—O—CO—) and the 2-(ethoxy)ethoxycarbonyl (CH₃CH₂—O—CH₂CH₂—O—CO—) radicals.

1-4C-Alkoxy-1-4C-alkoxycarbonylamino denotes an amino radical which is substituted by one of the abovementioned 1-4C-alkoxy-1-4C-alkoxycarbonyl radicals. Examples which may be mentioned are the 2-(methoxy)ethoxycarbonylamino and the 2-(ethoxy)ethoxycarbonylamino radicals.

2-4C-Alkenyloxy denotes a radical which, in addition to the oxygen atom, contains a 2-4C-alkenyl radical. An example which may be mentioned is the allyloxy radical.

Aryl-1-4C-alkyl denotes an aryl-substituted 1-4C-alkyl radical. An example which may be mentioned is the benzyl radical.

Aryl-1-4C-alkoxy denotes an aryl-substituted 1-4C-alkoxy radical. An example which may be mentioned is the benzyloxy radical.

Mono- or di-1-4C-alkylamino radicals contain, in addition to the nitrogen atom, one or two of the abovementioned 1-4C-alkyl radicals. Preference is given to di-1-4C-alkylamino and in particular to dimethyl-, diethyl- or diisopropylamino.

Mono- or di-1-4C-alkylamino-1-4C-alkyl denotes one of the abovementioned 1-4C-alkyl radicals which is substituted by one of the abovementioned mono- or di-1-4C-alkylamino radicals. Preferred mono- or di-1-4C-alkylamino-1-4C-alkyl radicals are the mono- or di-1-4C-alkylaminomethyl radicals. An Example which may be mentioned is the dimethylaminomethyl (CH₃)₂N—CH₂ radical.

1-4C-Alkylcarbonylamino denotes an amino group to which a 1-4C-alkylcarbonyl radical is attached. Examples which may be mentioned are the propionylamino (C₃H₇C(O)NH—) and the acetylamino (acetamido, CH₃C(O)NH—) radicals.

Imidazolyl denotes an imidazole, dihydroimidazole or an imidazolidine radical, tetrazolyl denotes a tetrazolyl, dihydrotetrazolyl or tetrazolidine radical and oxazolyl denotes an 1,3-oxazole, dihydro-1,3-oxazole or a 1,3-oxazolidine radical.

1-4C-alkylsulfonyl denotes a sulfonyl radical to which one of the abovementioned 1-4C-alkyl radicals is attached. Examples which may be mentioned are the methylsulfonyl CH₃—S(O₂)—, the CH₃CH₂—S(O₂)— ethylsulfonyl and the CH₃CH₂CH₂—S(O₂)— propylsulfonyl radicals.

Arylsulfonyl denotes a sulfonyl radical to which one of the abovementioned aryl radicals is attached. Examples which may be mentioned are the phenylsulfonyl C₆H₅—S(O₂)— or substituted phenylsulfonyl radicals.

Aryl-1-4C-alkylsulfonyl denotes a sulfonyl radical to which one of the abovementioned aryl-1-4C-alkyl radicals is attached. An example which may be mentioned is the benzylsulfonyl C₆H₅—CH₂—S(O₂)— radical.

Mono- or di-1-4C-alkylaminocarbonyl denotes a carbonyl radical to which a mono- or di-1-4C-alkylamino radical is attached. Examples which may be mentioned are the dimethylaminocarbonyl, diethylaminocarbonyl and diisopropylaminocarbonyl radicals.

Radicals Arom which may be mentioned are, for example, the following substitutents: 4-acetoxyphenyl, 4-acetamidophenyl, 2-methoxyphenyl, 3-methoxyphenyl, 4-methoxyphenyl, 3-benzyloxyphenyl, 4-benzyloxyphenyl, 3-benzyloxy-4-methoxyphenyl, 4-benzyloxy-3-methoxyphenyl, 3,5-bis(trifluoromethyl)phenyl, 4-butoxyphenyl, 2-chlorophenyl, 3-chlorophenyl, 4-chlorophenyl, 2-chloro-6-fluorophenyl, 3-chloro-4-fluorophenyl, 2-chloro-5-nitrophenyl, 4-chloro-3-nitrophenyl, 3-(4-chlorophenoxy)phenyl, 2,4-dichlorophenyl, 3,4-difluorophenyl, 2,4-dihydroxyphenyl, 2,6-dimethoxyphenyl, 3,4-dimethoxy-5-hydroxyphenyl, 2,5-dimethylphenyl, 3-ethoxy-4-hydroxyphenyl, 2-fluorophenyl, 4-fluorophenyl, 4-hydroxyphenyl, 2-hydroxy-5-nitrophenyl, 3-methoxy-2-nitrophenyl, 3-nitrophenyl, 2,3,5-trichlorophenyl, 2,4,6-trihydroxyphenyl, 2,3,4-trimethoxyphenyl, 2-hydroxy-1-naphthyl, 2-methoxy-1-naphthyl, 4-methoxy-1-naphthyl, 1-methyl-2-pyrrolyl, 2-pyrrolyl, 3-methyl-2-pyrrolyl, 3,4-dimethyl-2-pyrrolyl, 4-(2-methoxycarbonylethyl)-3-methyl-2-pyrrolyl, 5-ethoxycarbonyl-2,4-dimethyl-3-pyrrolyl, 3,4-dibromo-5-methyl-2-pyrrolyl, 2,5-dimethyl-1-phenyl-3-pyrrolyl, 5-carboxy-3-ethyl-4-methyl-2-pyrrolyl, 3,5-dimethyl-2-pyrrolyl, 2,5-dimethyl-1-(4-trifluoromethylphenyl)-3-pyrrolyl, 1-(2,6-dichloro-4-trifluoromethylphenyl)-2-pyrrolyl, 1-(2-nitrobenzyl)-2-pyrrolyl, 1-(2-fluorophenyl)-2-pyrrolyl, 1-(4-trifluoromethoxyphenyl)-2-pyrrolyl, 1-(2-nitrobenzyl)-2-pyrrolyl, 1-(4-ethoxycarbonyl)-2,5-dimethyl-3-pyrrolyl, 5-chloro-1,3-dimethyl-4-pyrazolyl, 5-chloro-1-methyl-3-trifluoromethyl-4-pyrazolyl, 1-(4-chlorobenzyl)-5-pyrazolyl, 1,3-dimethyl-5-(4-chlorophenoxy)-4-pyrazolyl, 1-methyl-3-trifluoromethyl-5-(3-trifluoromethylphenoxy)-4-pyrazolyl, 4-methoxycarbonyl-1-(2,6-dichlorophenyl)-5-pyrazolyl, 5-allyloxy-1-methyl-3-trifluoromethyl-4-pyrazolyl, 5-chloro-1-phenyl-3-trifluoromethyl-4-pyrazolyl, 3,5-dimethyl-1-phenyl-4-imidazolyl, 4-bromo-1-methyl-5-imidazolyl, 2-butylimidazolyl, 1-phenyl-1,2,3-triazol-4-yl, 3-indolyl, 4-indolyl, 7-indolyl, 5-methoxy-3-indolyl, 5-benzyloxy-3-indolyl, 1-benzyl-3-indolyl, 2-(4-chlorophenyl)-3-indolyl, 7-benzyloxy-3-indolyl, 6-benzyloxy-3-indolyl, 2-methyl-5-nitro-3-indolyl, 4,5,6,7-tetrafluoro-3-indolyl, 1-(3,5-difluorobenzyl)-3-indolyl, 1-methyl-2-(4-trifluorophenoxy)-3-indolyl, 1-methyl-2-benzimidazolyl, 5-nitro-2-furyl, 5-hydroxymethyl-2-furyl, 2-furyl, 3-furyl, 5-(2-nitro-4-trifluoromethylphenyl)-2-furyl, 4-ethoxycarbonyl-5-methyl-2-furyl, 5-(2-trifluoromethoxyphenyl)-2-furyl, 5-(4-methoxy-2-nitrophenyl)-2-furyl, 4-bromo-2-furyl, 5-dimethylamino-2-furyl, 5-bromo-2-furyl, 5-sulfo-2-furyl, 2-benzofuryl, 2-thienyl, 3-thienyl, 3-methyl-2-thienyl, 4-bromo-2-thienyl, 5-bromo-2-thienyl, 5-nitro-2-thienyl, 5-methyl-2-thienyl, 5-(4-methoxyphenyl)-2-thienyl, 4-methyl-2-thienyl, 3-phenoxy-2-thienyl, 5-carboxy-2-thienyl, 2,5-dichloro-3-thienyl, 3-methoxy-2-thienyl, 2-benzothienyl, 3-methyl-2-benzothienyl, 2-bromo-5-chloro-3-benzothienyl, 2-thiazolyl, 2-amino-4-chloro-5-thiazolyl, 2,4-dichloro-5-thiazolyl, 2-diethylamino-5-thiazolyl, 3-methyl-4-nitro-5-isoxazolyl, 2-pyridyl, 3-pyridyl, 4-pyridyl, 6-methyl-2-pyridyl, 3-hydroxy-5-hydroxymethyl-2-methyl-4-pyridyl, 2,6-dichloro-4-pyridyl, 3-chloro-5-trifluoromethyl-2-pyridyl, 4,6-dimethyl-2-pyridyl, 4-(4-chlorophenyl)-3-pyridyl, 2-chloro-5-methoxycarbonyl-6-methyl-4-phenyl-3-pyridyl, 2-chloro-3-pyridyl, 6-(3-trifluoromethylphenoxy)-3-pyridyl, 2-(4-chlorophenoxy)-3-pyridyl, 2,4-dimethoxy-5-pyrimidine, 2-quinolinyl, 3-quinolinyl, 4-quinolinyl, 2-chloro-3-quinolinyl, 2-chloro-6-methoxy-3-quinolinyl, 8-hydroxy-2-quinolinyl and 4-isoquinolinyl.

Suitable salts of compounds of the formula 1 are—depending on the substitution—in particular all acid addition salts. Particular mention may be made of the pharmacologically acceptable salts of the inorganic and organic acids customarily used in pharmacy. Those suitable are water-soluble and water-insoluble acid addition salts with acids such as, for example, hydrochloric acid, hydrobromic acid, phosphoric acid, nitric acid, sulfuric acid, acetic acid, citric acid, D-gluconic acid, benzoic acid, 2-(4-hydroxybenzoyl)benzoic acid, butyric acid, sulfosalicylic acid, maleic acid, lauric acid, malic acid, fumaric acid, succinic acid, oxalic acid, tartaric acid, embonic acid, stearic acid, toluenesulfonic acid, methanesulfonic acid or 3-hydroxy-2-naphthoic acid, where the acids are employed in the salt preparation in an equimolar ratio or in a ratio differing therefrom, depending on whether the acid is a mono- or polybasic acid and on which salt is desired.

Pharmacologically unacceptable salts, which can be initially obtained, for example, as process products in the preparation of the compounds according to the invention on an industrial scale, are converted into pharmacologically acceptable salts by processes known to the person skilled in the art.

It is known to the person skilled in the art that the compounds according to the invention and their salts can, for example when they are isolated in crystalline form, comprise varying amounts of solvents. The invention therefore also embraces all solvates and, in particular, all hydrates of the compounds of the formula 1, and all solvates and, in particular, all hydrates of the salts of the compounds of the formula 1.

The compounds of the formula 1 have at least one center of chirality in the skeleton. The invention thus provides all feasible enantiomers in any mixing ratio, including the pure enantiomers, which are the preferred subject matter of the invention.

One aspect of the invention (aspect a) relates to compounds of the formula 1, in which
R2 is hydrogen, 1-4C-alkyl, 3-7C-cycloalkyl, 3-7C-cycloalkyl-1-4C-alkyl, 1-4C-alkoxycarbonyl, hydroxy-1-4C-alkyl, halogen, 2-4C-alkenyl, 2-4C-alkynyl, fluoro-1-4C-alkyl or cyanomethyl,
R1, R3 and Arom have the meanings as indicated in the outset,
   and the salts of these compounds.

Another aspect of the invention (aspect b) relates to compounds of the formula 1, in which
R2 is hydroxy-3-4C-alkenyl, hydroxy-3-4C-alkinyl, hydroxy, 1-4C-alkoxy, amino, mono- or di-1-4C-alkylamino, 1-4C-alkylcarbonylamino, 1-4C-alkoxycarbonylamino, 1-4C-alkoxy-1-4C-alkoxycarbonylamino, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR21R22,
   where
   R21 is hydrogen, 1-7C-alkyl, hydroxy-1-4C-alkyl, 1-4C-alkoxy-1-4C-alkyl or 3-7C-cycloalkyl and
   R22 is hydrogen, 1-7C-alkyl, hydroxy-1-4C-alkyl, 1-4C-alkoxy-1-4C-alkyl or 3-7C-cycloalkyl,
   or where
   R21 and R22 together and including the nitrogen atom to which they are attached form a pyrrolidino, piperidino, morpholino, aziridino or azetidino radical,
   R1, R3 and Arom have the meanings as indicated in the outset,
      and the salts of these compounds.

Another aspect of the invention (aspect c) relates to compounds of the formula 1, in which
R3 is hydroxy-1-4C-alkyl, 1-4C-alkoxy-1-4C-alkyl, 1-4C-alkoxy-1-4C-alkoxy-1-4C-alkyl, 1-4C-alkoxycarbonyl, fluoro-1-4C-alkoxy-1-4C-alkyl or the radical —CO—NR31R32,
   where
   R31 is hydrogen, 1-7C-alkyl, hydroxy-1-4C-alkyl, 1-4C-alkoxy-1-4C-alkyl and
   R32 is hydrogen, 1-7C-alkyl, hydroxy-1-4C-alkyl, 1-4C-alkoxy-1-4C-alkyl,
   or where
   R31 and R32 together and including the nitrogen atom to which they are attached form a pyrrolidino, piperidino or morpholino radical,
R1, R2 and Arom have the meanings as indicated in the outset,
   and the salts of these compounds.

Another aspect of the invention (aspect d1) relates to compounds of the formula 1, in which
R3 is 1-4C-alkylcarbonyl, cyano, the radical —CO—NR31R32, the radical —SO₂—NR31R32, the radical —CS—NR31R32, the radical —C═N(OH)—NR31R32 or the group Het
   where for the radical —CO—NR31R32
   R31 is amino, hydroxy, 1-4-C-alkoxy, 3-7C-cycloalkyl, 1-4C-alkylsulfonyl, arylsulfonyl, aryl-1-4C-alkylsulfonyl, aryl and
   R32 is hydrogen, 1-7C-alkyl, hydroxy-1-4C-alkyl, 1-4C-alkoxy-1-4C-alkyl or 3-7C-cycloalkyl, and for the radicals —SO₂—NR31R32, —CS—NR31R32, and C═N(OH)—NR31R32
   R31 is hydrogen, amino, 1-7C-alkyl, hydroxy, hydroxy-1-4C-alkyl, 1-4C-alkoxy, 1-4C-alkoxy-1-4C-alkyl, 3-7C-cycloalkyl, 1-4C-alkylsulfonyl, arylsulfonyl, aryl-1-4C-alkylsulfonyl or aryl and
   R32 is hydrogen, 1-7C-alkyl, hydroxy-1-4C-alkyl, 1-4C-alkoxy-1-4C-alkyl or 3-7C-cycloalkyl,
   or where
   R31 and R32 together and including the nitrogen atom to which they are attached form a cyclic residue, substituted by R33, R34 and R35, selected from the group consisting of pyrrolidino, piperidino, piperazino, morpholino, aziridino or azetidino where in the case of pyrrolidino, piperidino, or morpholino, at least one of the substitutents R33, R34, or R35 has to be different from hydrogen, and
   Het is a heterocyclic residue, substituted by R33, R34 and R35, selected from the group consisting of oxadiazol, dihydrooxazol, dihydroimidazol, oxazol, imidazol, isoxazol, dihydroisoxazol, pyrazol, and tetrazol
   where
   R33 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R34 is hydrogen, 1-4C-alkyl, 1-4C-alkoxy, 1-4C-alkoxycarbonyl, halogen, trifluormethyl or hydroxy,
   R35 is hydrogen, 1-4C-alkyl, 1-4C-alkoxy, 1-4C-alkoxycarbonyl, halogen, trifluormethyl or hydroxy,
   where
   aryl is phenyl or substituted phenyl having one, two or three identical or different substitutents from the group consisting of 1-4C-alkyl, 1-4C-alkoxy, carboxyl, 1-4C-alkoxycarbonyl, halogen, trifluoromethyl, nitro, trifluoromethoxy, hydroxy and cyano,
R1, R2 and Arom have the meanings as indicated in the outset,
   and the salts of these compounds.

Another aspect of the invention (aspect d2) relates to compounds of the formula 1, in which
R3 is a imidazolyl, tetrazolyl or oxazolyl radical or the radical —CO—NR31R32,
   where
   R31 is 3-7C-cycloalkyl and
   R32 is hydrogen, 1-7C-alkyl, hydroxy-1-4C-alkyl, 1-4C-alkoxy-1-4C-alkyl or 3-7C-cycloalkyl,
   or where R31 and R32 together and including the nitrogen atom to which they are attached form a aziridino or azetidino radical,
R1, R2 and Arom have the meanings as indicated in the outset,
   and the salts of these compounds.

Another aspect of the invention (aspect e) relates to compounds of the formula 1, in which
R2 has the meaning according to aspect a,
R3 has the meaning according to aspect d1 or d2,
R1 and Arom have the meanings as indicated in the outset,
   and the salts of these compounds.

Another aspect of the invention (aspect f) relates to compounds of the formula 1, in which
R2 has the meaning according to aspect b,
R3 has the meaning according to aspect c,
R1 and Arom have the meanings as indicated in the outset,
   and the salts of these compounds.

Another aspect of the invention (aspect g) relates to compounds of the formula 1, in which
R2 has the meaning according to aspect b,
R3 has the meaning according to aspect d1 or d2,
R1 and Arom have the meanings as indicated in the outset,
   and the salts of these compounds.

A particular aspect of the invention (aspect h) relates to compounds of the formula 1, in which
Arom is phenyl
R1, R2 and R3 have the meanings as indicated in the outset.

The invention also relates to compounds of the formula 1,

where

R1 is hydrogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl or hydroxy-1-4C-alkyl,
R2 is hydrogen, 1-4C-alkyl, 3-7C-cycloalkyl, 3-7C-cycloalkyl-1-4C-alkyl, 1-4C-alkoxycarbonyl, hydroxy-1-4C-alkyl, hydroxy-3-4C-alkenyl, hydroxy-3-4C-alkinyl, halogen, 2-4C-alkenyl, 2-4C-alkynyl, fluoro-1-4C-alkyl, cyanomethyl, hydroxy, 1-4C-alkoxy, amino, mono- or di-1-4C-alkylamino, 1-4C-alkylcarbonylamino, 1-4C-alkoxycarbonylamino, 1-4C-alkoxy-1-4C-alkoxycarbonylamino, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR21R22,
   where
   R21 is hydrogen, 1-7C-alkyl, hydroxy-1-4C-alkyl, 1-4C-alkoxy-1-4C-alkyl or 3-7C-cycloalkyl and
   R22 is hydrogen, 1-7C-alkyl, hydroxy-1-4C-alkyl, 1-4C-alkoxy-1-4C-alkyl or 3-7C-cycloalkyl,
   or where
   R21 and R22 together and including the nitrogen atom to which they are attached form a pyrrolidino, piperidino, morpholino, aziridino or azetidino radical,
R3 is hydroxy-1-4C-alkyl, 1-4C-alkoxy-1-4C-alkyl, 1-4C-alkoxy-1-4C-alkoxy-1-4C-alkyl, 1-4C-alkoxycarbonyl, fluoro-1-4C-alkoxy-1-4C-alkyl, a imidazolyl, tetrazolyl or oxazolyl radical or the radical —CO—NR31R32,
   where
   R31 is hydrogen, 1-7C-alkyl, hydroxy-1-4C-alkyl, 1-4C-alkoxy-1-4C-alkyl or 3-7C-cycloalkyl and
   R32 is hydrogen, 1-7C-alkyl, hydroxy-1-4C-alkyl, 1-4C-alkoxy-1-4C-alkyl or 3-7C-cycloalkyl,
   or where
   R31 and R32 together and including the nitrogen atom to which they are attached form a pyrrolidino, piperidino, morpholino, aziridino or azetidino radical,
Arom is a R4-, R5-, R6- and R7-substituted mono- or bicyclic aromatic radical selected from the group consisting of phenyl, naphthyl, pyrrolyl, pyrazolyl, imidazolyl, 1,2,3-triazolyl, indolyl, benzimidazolyl, furanyl (furyl), benzofuranyl (benzofuryl), thiophenyl (thienyl), benzothiophenyl (benzothienyl), thiazolyl, isoxazolyl, pyridinyl, pyrimidinyl, quinolinyl and isoquinolinyl,
   where
   R4 is hydrogen, 1-4C-alkyl, hydroxy-1-4C-alkyl, 1-4C-alkoxy, 2-4C-alkenyloxy, 1-4C-alkylcarbonyl, carboxyl, 1-4C-alkoxycarbonyl, carboxy-1-4C-alkyl, 1-4C-alkoxycarbonyl-1-4C-alkyl, halogen, hydroxyl, aryl, aryl-1-4C-alkyl, aryloxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R5 is hydrogen, 1-4C-alkyl, 1-4C-alkoxy, 1-4C-alkoxycarbonyl, halogen, trifluoromethyl or hydroxyl,
   R6 is hydrogen, 1-4C-alkyl or halogen and
   R7 is hydrogen, 1-4C-alkyl or halogen,
   where
   aryl is phenyl or substituted phenyl having one, two or three identical or different substitutents from the group consisting of 1-4C-alkyl, 1-4C-alkoxy, carboxyl, 1-4C-alkoxycarbonyl, halogen, trifluoromethyl, nitro, trifluoromethoxy, hydroxyl and cyano,
with the proviso that,
when
R2 is hydrogen, 1-4C-alkyl, 3-7C-cycloalkyl, 3-7C-cycloalkyl-1-4C-alkyl, 1-4C-alkoxycarbonyl, hydroxy-1-4C-alkyl, halogen, 2-4C-alkenyl, 2-4C-alkynyl, fluoro-1-4C-alkyl or cyanomethyl,
then
R3 is a imidazolyl, tetrazolyl or oxazolyl radical or the radical —CO—NR31R32,
   where
   R31 is 3-7C-cycloalkyl and
   R32 is hydrogen, 1-7C-alkyl, hydroxy-1-4C-alkyl, 1-4C-alkoxy-1-4C-alkyl or 3-7C-cycloalkyl,
   or where R31 and R32 together and including the nitrogen atom to which they are attached form a aziridino or azetidino radical,
      and their salts.

Compounds of the formula 1 which are to be mentioned are those, where
R1 is hydrogen, 1-4C-alkyl, 3-7C-cycloalkyl
R2 is hydrogen, 1-4C-alkyl, 3-7C-cycloalkyl, 3-7C-cycloalkyl-1-4C-alkyl, 1-4C-alkoxycarbonyl, hydroxy-1-4C-alkyl, hydroxy-3-4C-alkenyl, hydroxy-3-4C-alkinyl, halogen, 2-4C-alkenyl, 2-4C-alkynyl, fluoro-1-4C-alkyl, cyanomethyl, hydroxy, 1-4C-alkoxy, amino, mono- or di-1-4C-alkylamino, 1-4C-alkylcarbonylamino, 1-4C-alkoxycarbonylamino, 1-4C-alkoxy-1-4C-alkoxycarbonylamino, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR21R22,
   where
   R21 is hydrogen, 1-7C-alkyl, hydroxy-1-4C-alkyl, 1-4C-alkoxy-1-4C-alkyl or 3-7C-cycloalkyl and
   R22 is hydrogen, 1-7C-alkyl, hydroxy-1-4C-alkyl, 1-4C-alkoxy-1-4C-alkyl or 3-7C-cycloalkyl,
   or where
   R21 and R22 together and including the nitrogen atom to which they are attached form a pyrrolidino, piperidino, morpholino, aziridino or azetidino radical,
R3 is hydroxy-1-4C-alkyl, 1-4C-alkoxy-1-4C-alkyl, 1-4C-alkoxy-1-4C-alkoxy-1-4C-alkyl, 1-4C-alkoxycarbonyl, fluoro-1-4C-alkoxy-1-4C-alkyl, a imidazolyl, tetrazolyl or oxazolyl radical or the radical —CO—NR31R32,
   where
   R31 is hydrogen, 1-7C-alkyl, hydroxy-1-4C-alkyl, 1-4C-alkoxy-1-4C-alkyl or 3-7C-cycloalkyl and
   R32 is hydrogen, 1-7C-alkyl, hydroxy-1-4C-alkyl, 1-4C-alkoxy-1-4C-alkyl or 3-7C-cycloalkyl,
   or where
   R31 and R32 together and including the nitrogen atom to which they are attached form a pyrrolidino, piperidino, morpholino, aziridino or azetidino radical,
Arom is a R4-, R5-, R6- and R7-substituted phenyl
   where
   R4 is hydrogen or 1-4C-alkyl, halogen, 1-4C-alkoxy, trifluoromethyl
   R5 is hydrogen or 1-4C-alkyl, halogen
   R6 is hydrogen and
   R7 is hydrogen
with the proviso that,
when
R2 is hydrogen, 1-4C-alkyl, 3-7C-cycloalkyl, 3-7C-cycloalkyl-1-4C-alkyl, 1-4C-alkoxycarbonyl, hydroxy-1-4C-alkyl, halogen, 2-4C-alkenyl, 2-4C-alkynyl, fluoro-1-4C-alkyl or cyanomethyl,
then
R3 is a imidazolyl, tetrazolyl or oxazolyl radical or the radical —CO—NR31R32,
   where
   R31 is 3-7C-cycloalkyl and
   R32 is hydrogen, 1-7C-alkyl, hydroxy-1-4C-alkyl, 1-4C-alkoxy-1-4C-alkyl or 3-7C-cycloalkyl,
   or where R31 and R32 together and including the nitrogen atom to which they are attached form a aziridino or azetidino radical,
      and their salts.

Particular mention may be made of those compounds of the formula 1, where
R1 is hydrogen, 1-4C-alkyl or 3-7C-cycloalkyl,
R2 is hydrogen, 1-4C-alkyl, hydroxy-3-4C-alkenyl, hydroxy-3-4C-alkinyl, hydroxy, 1-4C-alkoxy, amino, mono- or di-1-4C-alkylamino, 1-4C-alkylcarbonylamino, 1-4C-alkoxycarbonylamino, 1-4C-alkoxy-1-4C-alkoxycarbonylamino, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR21R22,
   where
   R21 is hydrogen, 1-7C-alkyl, hydroxy-1-4C-alkyl, 1-4C-alkoxy-1-4C-alkyl or 3-7C-cycloalkyl and
   R22 is hydrogen, 1-7C-alkyl, hydroxy-1-4C-alkyl, 1-4C-alkoxy-1-4C-alkyl or 3-7C-cycloalkyl,
   or where
   R21 and R22 together and including the nitrogen atom to which they are attached form a pyrrolidino, piperidino, morpholino, aziridino or azetidino radical,
R3 is hydroxy-1-4C-alkyl, 1-4C-alkoxy-1-4C-alkyl, 1-4C-alkoxy-1-4C-alkoxy-1-4C-alkyl, 1-4C-alkoxycarbonyl, fluoro-1-4C-alkoxy-1-4C-alkyl, a imidazolyl, tetrazolyl or oxazolyl radical or the radical —CO—NR31R32,
   where
   R31 is hydrogen, 1-7C-alkyl, hydroxy-1-4C-alkyl, 1-4C-alkoxy-1-4C-alkyl or 3-7C-cycloalkyl and
   R32 is hydrogen, 1-7C-alkyl, hydroxy-1-4C-alkyl, 1-4C-alkoxy-1-4C-alkyl or 3-7C-cycloalkyl,
   or where
   R31 and R32 together and including the nitrogen atom to which they are attached form a pyrrolidino, piperidino, morpholino, aziridino or azetidino radical,
Arom is a R4-, R5-, R6- and R7-substituted phenyl
   where
   R4 is hydrogen or 1-4C-alkyl, halogen, 1-4C-alkoxy, trifluoromethyl
   R5 is hydrogen or 1-4C-alkyl, halogen
   R6 is hydrogen and
   R7 is hydrogen
with the proviso that,
when
R2 is hydrogen or 1-4C-alkyl,
then
R3 is a imidazolyl, tetrazolyl or oxazolyl radical or the radical —CO—NR31R32,
   where
   R31 is 3-7C-cycloalkyl and
   R32 is hydrogen, 1-7C-alkyl, hydroxy-1-4C-alkyl, 1-4C-alkoxy-1-4C-alkyl or 3-7C-cycloalkyl,
   or where R31 and R32 together and including the nitrogen atom to which they are attached form a aziridino or azetidino radical,
      and their salts.

Emphasis is given to compounds of the formula 1, where
R1 is 1-4C-alkyl,
R2 is hydroxy-3-4C-alkenyl, hydroxy-3-4C-alkinyl, hydroxy, 1-4C-alkoxy, amino, mono- or di-1-4C-alkylamino, 1-4C-alkylcarbonylamino, 1-4C-alkoxycarbonylamino, 1-4C-alkoxy-1-4C-alkoxycarbonylamino, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR21R22,
   where
   R21 is hydrogen, 1-4C-alkyl, hydroxy-1-4C-alkyl, 1-4C-alkoxy-1-4C-alkyl or 3-7C-cycloalkyl and
   R22 is hydrogen, 1-4C-alkyl, hydroxy-1-4C-alkyl, 1-4C-alkoxy-1-4C-alkyl or 3-7C-cycloalkyl,
   or where
   R21 and R22 together and including the nitrogen atom to which they are attached form a pyrrolidino, piperidino, morpholino, aziridino or azetidino radical,
R3 is a imidazolyl, tetrazolyl or oxazolyl radical or the radical —CO—NR31R32,
   where
   R31 is hydrogen, 1-4C-alkyl or 3-7C-cycloalkyl
   R32 is hydrogen, 1-4C-alkyl or 3-7C-cycloalkyl,
   or where
   R31 and R32 together and including the nitrogen atom to which they are attached form a pyrrolidino, piperidino, morpholino, aziridino or azetidino radical,
Arom is phenyl
   and their salts.

Emphasis is also given to compounds of the formula 1, where
R1 is 1-4C-alkyl
R2 is hydroxy-3-4C-alkenyl, hydroxy-3-4C-alkinyl, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR21R22,
   where
   R21 is hydrogen, 1-4C-alkyl or 1-4C-alkoxy-1-4C-alkyl and
   R22 is hydrogen, 1-4C-alkyl or 1-4C-alkoxy-1-4C-alkyl,
R3 is the radical —CO—NR31R32,
   where
   R31 is 1-4C-alkyl and
   R32 is 1-4C-alkyl
Arom is Phenyl
   and their salts.

Emphasis is also given to compounds of the formula 1, where
R1 is 1-4C-alkyl,
R2 is 1-4C-alkyl,
R3 is a imidazolyl, tetrazolyl or oxazolyl radical or the radical —CO—NR31R32,
   where
   R31 is 3-7C-cycloalkyl
   R32 is hydrogen, 1-4C-alkyl or 3-7C-cycloalkyl,
   or where
   R31 and R32 together and including the nitrogen atom to which they are attached form a pyrrolidino, piperidino, morpholino, aziridino or azetidino radical,
Arom is phenyl,
   and their salts.

Compounds of the formula 1 which are also to be mentioned are those, where
R1 is hydrogen, 1-4C-alkyl, 3-7C-cycloalkyl
R2 is hydrogen, 1-4C-alkyl, 3-7C-cycloalkyl, 3-7C-cycloalkyl-1-4C-alkyl, 1-4C-alkoxycarbonyl, hydroxy-1-4C-alkyl, hydroxy-3-4C-alkenyl, hydroxy-3-4C-alkinyl, halogen, 2-4C-alkenyl, 2-4C-alkynyl, fluoro-1-4C-alkyl, cyanomethyl, hydroxy, 1-4C-alkoxy, amino, mono- or di-1-4C-alkylamino, 1-4C-alkylcarbonylamino, 1-4C-alkoxycarbonylamino, 1-4C-alkoxy-1-4C-alkoxycarbonylamino, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR21R22,
   where
   R21 is hydrogen, 1-7C-alkyl, hydroxy-1-4C-alkyl, 1-4C-alkoxy-1-4C-alkyl or 3-7C-cycloalkyl and
   R22 is hydrogen, 1-7C-alkyl, hydroxy-1-4C-alkyl, 1-4C-alkoxy-1-4C-alkyl or 3-7C-cycloalkyl,
   or where
   R21 and R22 together and including the nitrogen atom to which they are attached form a pyrrolidino, piperidino, morpholino, aziridino or azetidino radical,
R3 is 1-4C-alkylcarbonyl, hydroxy-1-4C-alkyl, 1-4C-alkoxy-1-4C-alkyl, 1-4C-alkoxy-1-4C-alkoxy-1-4C-alkyl, 1-4C-alkoxycarbonyl, fluoro-1-4C-alkoxy-1-4C-alkyl, cyano, the radical —CO—NR31R32, the radical —SO₂—NR31R32, the radical —CS—NR31R32, the radical C═N(OH)—NR31R32 or the group Het
   where
   R31 is hydrogen, amino, 1-7C-alkyl, hydroxy, hydroxy-1-4C-alkyl, 1-4C-alkoxy, 1-4C-alkoxy-1-4C-alkyl or 3-7C-cycloalkyl, 1-4C-alkylsulfonyl, arylsulfonyl, aryl-1-4C-alkylsulfonyl, aryl and
   R32 is hydrogen, 1-7C-alkyl, hydroxy-1-4C-alkyl, 1-4C-alkoxy-1-4C-alkyl or 3-7C-cycloalkyl,
   or where
   R31 and R32 together and including the nitrogen atom to which they are attached form a cyclic residue, substituted by R33, R34 and R35, selected from the group consisting of pyrrolidino, piperidino, piperazino, morpholino, aziridino or azetidino, and
   Het is a heterocyclic residue, substituted by R33, R34 and R35, selected from the group consisting of oxadiazol, dihydrooxazol, dihydroimidazol, oxazol, imidazol, isoxazol, dihydroisoxazol, pyrazol, and tetrazol
   where
   R33 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R34 is hydrogen, 1-4C-alkyl, 1-4C-alkoxy, 1-4C-alkoxycarbonyl, halogen, trifluormethyl or hydroxy,
   R35 is hydrogen, 1-4C-alkyl, 1-4C-alkoxy, 1-4C-alkoxycarbonyl, halogen, trifluormethyl or hydroxy,
   where
   aryl is phenyl or substituted phenyl having one, two or three identical or different substitutents from the group consisting of 1-4C-alkyl, 1-4C-alkoxy, carboxyl, 1-4C-alkoxycarbonyl, halogen, trifluoromethyl, nitro, trifluoromethoxy, hydroxy and cyano,
Arom is a R4- and R5-substituted phenyl, pyrrolyl, furanyl (furyl), thiophenyl (thienyl)
   where
   R4 is hydrogen or 1-4C-alkyl, halogen, 1-4C-alkoxy, trifluoromethyl
   R5 is hydrogen or 1-4C-alkyl, halogen
with the proviso that,
when
R2 is hydrogen, 1-4C-alkyl, 3-7C-cycloalkyl, 3-7C-cycloalkyl-1-4C-alkyl, 1-4C-alkoxycarbonyl, hydroxy-1-4C-alkyl, halogen, 2-4C-alkenyl, 2-4C-alkynyl, fluoro-1-4C-alkyl or cyanomethyl,
then
R3 is 1-4C-alkylcarbonyl, cyano, the radical —CO—NR31R32, the radical —SO₂—NR31R32, the radical —NR31R32, the radical C═N(OH)—NR31R32 or the group Het
   where for the radical —CO—NR31R32
   R31 is amino, hydroxy, 1-4C-alkoxy, 3-7C-cycloalkyl, 1-4C-alkylsulfonyl, arylsulfonyl, aryl-1-4C-alkylsulfonyl, aryl and
   R32 is hydrogen, 1-7C-alkyl, hydroxy-1-4C-alkyl, 1-4C-alkoxy-1-4C-alkyl or 3-7C-cycloalkyl, and for the radicals —SO₂—NR31R32, —CS—NR31R32, and C═N(OH)—NR31R32
   R31 is hydrogen, amino, 1-7C-alkyl, hydroxy, hydroxy-1-4C-alkyl, 1-4C-alkoxy, 1-4C-alkoxy-1-4C-alkyl or 3-7C-cycloalkyl, 1-4C-alkylsulfonyl, arylsulfonyl, aryl-1-4C-alkylsulfonyl, aryl and
   R32 is hydrogen, 1-7C-alkyl, hydroxy-1-4C-alkyl, 1-4C-alkoxy-1-4C-alkyl or 3-7C-cycloalkyl,
   or where
   R31 and R32 together and including the nitrogen atom to which they are attached form a cyclic residue, substituted by R33, R34 and R35, selected from the group consisting of pyrrolidino, piperidino, piperazino, morpholino, aziridino or azetidino where in the case of pyrrolidino, piperidino, or morpholino, at least one of the substitutents R33, R34, or R35 has to be different from hydrogen, and
   Het is a heterocyclic residue, substituted by R33, R34 and R35, selected from the group consisting of oxadiazol, dihydrooxazol, dihydroimidazol, oxazol, imidazol, isoxazol, dihydroisoxazol, pyrazol, and tetrazol
   where
   R33 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R34 is hydrogen, 1-4C-alkyl, 1-4C-alkoxy, 1-4C-alkoxycarbonyl, halogen, trifluormethyl or hydroxy,
   R35 is hydrogen, 1-4C-alkyl, 1-4C-alkoxy, 1-4C-alkoxycarbonyl, halogen, trifluormethyl or hydroxy,
   where
   aryl is phenyl or substituted phenyl having one, two or three identical or different substitutents from the group consisting of 1-4C-alkyl, 1-4C-alkoxy, carboxyl, 1-4C-alkoxycarbonyl, halogen, trifluoromethyl, nitro, trifluoromethoxy, hydroxy and cyano,
      and their salts.

Particular mention may also be made of those compounds of the formula 1, where
R1 is hydrogen, 1-4C-alkyl or 3-7C-cycloalkyl,
R2 is hydrogen, 1-4C-alkyl, hydroxy-3-4C-alkenyl, hydroxy-3-4C-alkinyl, hydroxy, 1-4C-alkoxy, amino, mono- or di-1-4C-alkylamino, 1-4C-alkylcarbonylamino, 1-4C-alkoxycarbonylamino, 1-4C-alkoxy-1-4C-alkoxycarbonylamino, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR21R22,
   where
   R21 is hydrogen, 1-7C-alkyl, hydroxy-1-4C-alkyl, 1-4C-alkoxy-1-4C-alkyl or 3-7C-cycloalkyl and
   R22 is hydrogen, 1-7C-alkyl, hydroxy-1-4C-alkyl, 1-4C-alkoxy-1-4C-alkyl or 3-7C-cycloalkyl,
   or where
   R21 and R22 together and including the nitrogen atom to which they are attached form a pyrrolidino, piperidino, morpholino, aziridino or azetidino radical,
R3 is 1-4C-alkylcarbonyl, hydroxy-1-4C-alkyl, 1-4C-alkoxy-1-4C-alkyl, 1-4C-alkoxy-1-4C-alkoxy-1-4C-alkyl, 1-4C-alkoxycarbonyl, fluoro-1-4C-alkoxy-1-4C-alkyl, cyano, the radical —CO—NR31R32, the radical —SO₂—NR31R32, the radical CS—NR31R32, the radical C═N(OH)—NR31R32 or the group Het
   where
   R31 is hydrogen, amino, 1-7C-alkyl, hydroxy, hydroxy-1-4C-alkyl, 1-4C-alkoxy, 1-4C-alkoxy-1-4C-alkyl, 3-7C-cycloalkyl, 1-4C-alkylsulfonyl, arylsulfonyl, aryl-1-4C-alkylsulfonyl or aryl and
   R32 is hydrogen, 1-7C-alkyl, hydroxy-1-4C-alkyl, 1-4C-alkoxy-1-4C-alkyl or 3-7C-cycloalkyl,
   or where
   R31 and R32 together and including the nitrogen atom to which they are attached form a cyclic residue, substituted by R33, R34 and R35, selected from the group consisting of pyrrolidino, piperidino, piperazino, morpholino, aziridino or azetidino, and
   Het is a heterocyclic residue, substituted by R33, R34 and R35, selected from the group consisting of oxadiazol, dihydrooxazol, dihydroimidazol, oxazol, imidazol, isoxazol, dihydroisoxazol, pyrazol, and tetrazol
   where
   R33 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R34 is hydrogen, 1-4C-alkyl, 1-4C-alkoxy, 1-4C-alkoxycarbonyl, halogen, trifluormethyl or hydroxy,
   R35 is hydrogen, 1-4C-alkyl, 1-4C-alkoxy, 1-4C-alkoxycarbonyl, halogen, trifluormethyl or hydroxy,
   where
   aryl is phenyl or substituted phenyl having one, two or three identical or different substitutents from the group consisting of 1-4C-alkyl, 1-4C-alkoxy, carboxyl, 1-4C-alkoxycarbonyl, halogen, trifluoromethyl, nitro, trifluoromethoxy, hydroxy and cyano,
Arom is a R4- and R5-substituted phenyl, pyrrolyl, furanyl (furyl), thiophenyl (thienyl)
   where
   R4 is hydrogen or 1-4C-alkyl, halogen, 1-4C-alkoxy, trifluoromethyl
   R5 is hydrogen or 1-4C-alkyl, halogen
with the proviso that,
when
R2 is hydrogen or 1-4C-alkyl,
then
R3 is 1-4C-alkylcarbonyl, cyano, the radical —CO—NR31R32, the radical —SO₂—NR31R32, the radical —CS—NR31R32, the radical C═N(OH)—NR31R32 or the group Het
   where for the radical —CO—NR31R32
   R31 is amino, hydroxy, 1-4C-alkoxy, 3-7C-cycloalkyl, 1-4C-alkylsulfonyl, arylsulfonyl, aryl-1-4C-alkylsulfonyl, aryl and
   R32 is hydrogen, 1-7C-alkyl, hydroxy-1-4C-alkyl, 1-4C-alkoxy-1-4C-alkyl or 3-7C-cycloalkyl, and for the radicals —SO₂—NR31R32, —CS—NR31R32, and C═N(OH)—NR31R32
   R31 is hydrogen, amino, 1-7C-alkyl, hydroxy, hydroxy-1-4C-alkyl, 1-4C-alkoxy, 1-4C-alkoxy-1-4C-alkyl, 3-7C-cycloalkyl, 1-4C-alkylsulfonyl, arylsulfonyl, aryl-1-4C-alkylsulfonyl or aryl and
   R32 is hydrogen, 1-7C-alkyl, hydroxy-1-4C-alkyl, 1-4C-alkoxy-1-4C-alkyl or 3-7C-cycloalkyl,
   or where
   R31 and R32 together and including the nitrogen atom to which they are attached form a cydic residue, substituted by R33, R34 and R35, selected from the group consisting of pyrrolidino, piperidino, piperazino, morpholino, aziridino or azetidino where in the case of pyrrolidino, piperidino, or morpholino, at least one of the substitutents R33, R34, or R35 has to be different from hydrogen, and
   Het is a heterocyclic residue, substituted by R33, R34 and R35, selected from the group consisting of oxadiazol, dihydrooxazol, dihydroimidazol, oxazol, imidazol, isoxazol, dihydroisoxazol, pyrazol, and tetrazol
   where
   R33 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R34 is hydrogen, 1-4C-alkyl, 1-4C-alkoxy, 1-4C-alkoxycarbonyl, halogen; trifluormethyl or hydroxy,
   R35 is hydrogen, 1-4C-alkyl, 1-4C-alkoxy, 1-4C-alkoxycarbonyl, halogen, trifluormethyl or hydroxy,
   where
   aryl is phenyl or substituted phenyl having one, two or three identical or different substitutents from the group consisting of 1-4C-alkyl, 1-4C-alkoxy, carboxyl, 1-4C-alkoxycarbonyl, halogen, trifluoromethyl, nitro, trifluoromethoxy, hydroxy and cyano,
      and their salts.

Emphasis is also given to compounds of the formula 1, where
R1 is 1-4C-alkyl,
R2 is 1-4C-alkyl, hydroxy-3-4C-alkenyl, hydroxy-3-4C-alkinyl, hydroxy, 1-4C-alkoxy, amino, mono- or di-1-4C-alkylamino, 1-4C-alkylcarbonylamino, 1-4C-alkoxycarbonylamino, 1-4C-alkoxy-1-4C-alkoxycarbonylamino, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR21R22,
   where
   R21 is hydrogen, 1-4C-alkyl, hydroxy-1-4C-alkyl, 1-4C-alkoxy-1-4C-alkyl or 3-7C-cycloalkyl and
   R22 is hydrogen, 1-4C-alkyl, hydroxy-1-4C-alkyl, 1-4C-alkoxy-1-4C-alkyl or 3-7C-cycloalkyl,
   or where
   R21 and R22 together and including the nitrogen atom to which they are attached form a pyrrolidino, piperidino, morpholino, aziridino or azetidino radical,
R3 is cyano, the radical —CO—NR31R32, the radical —CS—NR31R32, or the group Het
   where
   R31 is hydrogen, 1-7C-alkyl, 1-4C-alkoxy, 3-7C-cycloalkyl, 1-4C-alkylsulfonyl, arylsulfonyl, aryl-1-4C-alkylsulfonyl, aryl and
   R32 is hydrogen, 1-7C-alkyl, or 3-7C-cycloalkyl,
   or where
   R31 and R32 together and including the nitrogen atom to which they are attached form a cyclic residue, substituted by R33, selected from the group consisting of pyrrolidino, piperidino, piperazino, morpholino, aziridino or azetidino, and
   Het is a heterocyclic residue, substituted by R33, selected from the group consisting of oxadiazol, dihydrooxazol, dihydroimidazol, oxazol, imidazol, isoxazol, dihydroisoxazol, pyrazol, and tetrazol
   where
   R33 is hydrogen, 1-4C-alkyl, 1-4C-alkoxy, 1-4C-alkylcarbonyl, 1-4C-alkoxycarbonyl, halogen, hydroxy
   where
   aryl is phenyl or substituted phenyl having one, two or three identical or different substitutents from the group consisting of 1-4C-alkyl, 1-4C-alkoxy, carboxyl, 1-4C-alkoxycarbonyl, halogen, trifluoromethyl, nitro, trifluoromethoxy, hydroxy and cyano,
Arom is a R4- and R5-substituted phenyl, pyrrolyl, furanyl (furyl), thiophenyl (thienyl)
   where
   R4 is hydrogen or 1-4C-alkyl, halogen, 1-4C-alkoxy, trifluoromethyl
   R5 is hydrogen or 1-4C-alkyl, halogen
with the proviso that,
when
R2 is 1-4C-alkyl,
then
R3 is cyano, the radical —CO—NR31R32, the radical —CS—NR31R32, or the group Het
   where for the radical —CO—NR31R32
   R31 is 1-4C-alkoxy, 3-7C-cycloalkyl, 1-4C-alkylsulfonyl, arylsulfonyl, aryl-1-4C-alkylsulfonyl, aryl and
   R32 is hydrogen, 1-7C-alkyl, or 3-7C-cycloalkyl,
   and for the radical —CS—NR31R32
   R31 is hydrogen, 1-7C-alkyl, 1-4C-alkoxy, 3-7C-cycloalkyl, 1-4C-alkylsulfonyl, arylsulfonyl, aryl-1-4C-alkylsulfonyl, aryl and
   R32 is hydrogen, 1-7C-alkyl, or 3-7C-cycloalkyl,
   or where
   R31 and R32 together and including the nitrogen atom to which they are attached form a cyclic residue, substituted by R33, selected from the group consisting of pyrrolidino, piperidino, piperazino, morpholino, aziridino or azetidino where in the case of pyrrolidino, piperidino, or morpholino, the substitutent R33 has to be different from hydrogen, and
   Het is a heterocyclic residue, substituted by R33, selected from the group consisting of oxadiazol, dihydrooxazol, dihydroimidazol, oxazol, imidazol, isoxazol, dihydroisoxazol, pyrazol, and tetrazol
   where
   R33 is hydrogen, 1-4C-alkyl, 1-4C-alkoxy, 1-4C-alkylcarbonyl, 1-4C-alkoxycarbonyl, halogen, hydroxy,
   where
   aryl is phenyl or substituted phenyl having one, two or three identical or different substitutents from the group consisting of 1-4C-alkyl, 1-4C-alkoxy, carboxyl, 1-4C-alkoxycarbonyl, halogen, trifluoromethyl, nitro, trifluoromethoxy, hydroxy and cyano,
      and their salts.

Emphasis is also given to compounds of the formula 1, where
R1 is 1-4C-alkyl,
R2 is 1-4C-alkyl, hydroxy-3-4C-alkenyl, hydroxy-3-4C-alkinyl, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR21R22,
   where
   R21 is hydrogen, 1-4C-alkyl, 1-4C-alkoxy-1-4C-alkyl and
   R22 is hydrogen, 1-4C-alkyl, 1-4C-alkoxy-1-4C-alkyl,
R3 is cyano, the radical —CO—NR31R32, the radical —CS—NR31R32, or the group Het
   where
   R31 is hydrogen, 1-7C-alkyl, 1-4C-alkoxy, 3-7C-cycloalkyl, 1-4C-alkylsulfonyl, arylsulfonyl, aryl-1-4C-alkylsulfonyl, aryl and
   R32 is hydrogen, 1-7C-alkyl, or 3-7C-cycloalkyl,
   or where
   R31 and R32 together and including the nitrogen atom to which they are attached form a cyclic residue, substituted by R33, selected from the group consisting of pyrrolidino, piperazino, aziridino or azetidino, and
   Het is a heterocyclic residue, substituted by R33, selected from the group consisting of dihydrooxazol, dihydroimidazol, oxazol, imidazol, isoxazol, dihydroisoxazol, and tetrazol
   where
   R33 is hydrogen, 1-4C-alkyl, 1-4C-alkoxy, 1-4C-alkylcarbonyl, 1-4C-alkoxycarbonyl, halogen, hydroxy
   where
   aryl is phenyl or substituted phenyl having one, two or three identical or different substitutents from the group consisting of 1-4C-alkyl, 1-4C-alkoxy, 1-4C-alkoxycarbonyl, halogen, hydroxy,
Arom is a R4-substituted phenyl, pyrrolyl, furanyl (furyl), thiophenyl (thienyl)
   where
   R4 is hydrogen or 1-4C-alkyl, halogen, 1-4C-alkoxy, trifluoromethyl
with the proviso that,
when
R2 is 1-4C-alkyl,
then
R3 is cyano, the radical —CO—NR31R32, the radical CS—NR31R32, or the group Het
   where for —CO—NR31R32
   R31 is 1-4C-alkoxy, 3-7C-cycloalkyl, 1-4C-alkylsulfonyl, arylsulfonyl, aryl-1-4C-alkylsulfonyl, aryl and
   R32 is hydrogen, 1-7C-alkyl, or 3-7C-cycloalkyl,
   and for CS—NR31R32
   R31 is hydrogen, 1-7C-alkyl, 1-4C-alkoxy, 3-7C-cycloalkyl, 1-4C-alkylsulfonyl, arylsulfonyl, aryl-1-4C-alkylsulfonyl, aryl and
   R32 is hydrogen, 1-7C-alkyl, or 3-7C-cycloalkyl,
   or where
   R31 and R32 together and including the nitrogen atom to which they are attached form a cyclic residue, substituted by R33, selected from the group consisting of pyrrolidino, piperazino, aziridino or azetidino where in the case of pyrrolidino, the substitutent R33 has to be different from hydrogen, and
   Het is a heterocyclic residue, substituted by R33, selected from the group consisting of dihydrooxazol, dihydroimidazol, oxazol, imidazol, isoxazol, dihydroisoxazol, and tetrazol
   where
   R33 is hydrogen, 1-4C-alkyl, 1-4C-alkoxy, 1-4C-alkylcarbonyl, 1-4C-alkoxycarbonyl, halogen, hydroxy
   where
   aryl is phenyl or substituted phenyl having one, two or three identical or different substitutents from the group consisting of 1-4C-alkyl, 1-4C-alkoxy, 1-4C-alkoxycarbonyl, halogen, hydroxy,
      and their salts.

Emphasis is also given to compounds of the formula 1, where
R1 is 1-4C-alkyl
R2 is hydroxy-3-4C-alkenyl, hydroxy-3-4C-alkinyl, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR21R22,
   where
   R21 is hydrogen, 1-4C-alkyl or 1-4C-alkoxy-1-4C-alkyl and
   R22 is hydrogen, 1-4C-alkyl or 1-4C-alkoxy-1-4C-alkyl,
R3 is cyano, the radical —CO—NR31R32, the radical —CS—NR31R32, or the group Het
   where
   R31 is hydrogen, 1-7C-alkyl, 1-4C-alkoxy, 3-7C-cycloalkyl, 1-4C-alkylsulfonyl, arylsulfonyl, aryl-1-4C-alkylsulfonyl, aryl and
   R32 is hydrogen, 1-7C-alkyl, or 3-7C-cycloalkyl,
   or where
   R31 and R32 together and including the nitrogen atom to which they are attached form a cyclic residue, substituted by R33, selected from the group consisting of pyrrolidino, piperazino, aziridino or azetidino, and
   Het is a heterocyclic residue, substituted by R33, selected from the group consisting of dihydrooxazol, dihydroimidazol, oxazol, imidazol, isoxazol, dihydroisoxazol, and tetrazol
   where
   R33 is hydrogen, 1-4C-alkyl, 1-4C-alkoxy, 1-4C-alkylcarbonyl, 1-4C-alkoxycarbonyl, halogen, hydroxy
   where
   aryl is phenyl or substituted phenyl having one, two or three identical or different substitutents from the group consisting of 1-4C-alkyl, 1-4C-alkoxy, 1-4C-alkoxycarbonyl, halogen, hydroxy,
Arom is a R4-substituted phenyl, pyrrolyl, furanyl (furyl), thiophenyl (thienyl)
   where
   R4 is hydrogen or 1-4C-alkyl, halogen, 1-4C-alkoxy, trifluoromethyl
      and their salts.

Emphasis is also given to compounds of the formula 1, where
R1 is 1-4C-alkyl,
R2 is 1-4C-alkyl,
R3 is cyano, the radical —CO—NR31R32, the radical —CS—NR31R32, or the group Het
   where for the radical —CO—NR31R32
   R31 1-4-C-alkoxy, 3-7C-cycloalkyl, 1-4C-alkylsulfonyl, arylsulfonyl, aryl-1-4C-alkylsulfonyl, aryl and
   R32 is hydrogen, 1-7C-alkyl, or 3-7C-cycloalkyl,
   for the radical —CS—NR31R32
   R31 is hydrogen, 1-7C-alkyl, 1-4C-alkoxy, 3-7C-cycloalkyl, 1-4C-alkylsulfonyl, arylsulfonyl, aryl-1-4C-alkylsulfonyl, aryl and
   R32 is hydrogen, 1-7C-alkyl, or 3-7C-cycloalkyl,
   or where
   R31 and R32 together and including the nitrogen atom to which they are attached form a cyclic residue, substituted by R33, selected from the group consisting of pyrrolidino, piperazino, aziridino or azetidino where in the case of pyrrolidino, the substitutent R33 has to be different from hydrogen, and
   Het is a heterocyclic residue, substituted by R33, selected from the group consisting of dihydrooxazol, dihydroimidazol, oxazol, imidazol, isoxazol, dihydroisoxazol, and tetrazol
   where
   R33 is hydrogen, 1-4C-alkyl, 1-4C-alkoxy, 1-4C-alkylcarbonyl, 1-4C-alkoxycarbonyl,
   where
   aryl is phenyl or substituted phenyl having one, two or three identical or different substitutents from the group consisting of 1-4C-alkyl, 1-4C-alkoxy, 1-4C-alkoxycarbonyl, halogen, hydroxy,
Arom is a R4-substituted phenyl, pyrrolyl, furanyl (furyl), thiophenyl (thienyl)
   where
   R4 is hydrogen or 1-4C-alkyl, halogen, 1-4C-alkoxy, trifluoromethyl
      and their salts.

Particular emphasis is also given to compounds of the formula 1, where
R1 is 1-4C-alkyl,
R2 is 1-4C-alkyl, hydroxy-3-4C-alkinyl, carboxyl, mono- or di-1-4C-alkylamino-1-4C-alkyl, 1-4C-alkylcarbonyl, 2-4C-alkinylcarbonyl or the radical —CO—NR21R22,
   where
   R21 is 1-4C-alkyl or 1-4C-alkoxy-1-4C-alkyl and
   R22 is hydrogen or 1-4C-alkyl,
R3 is cyano, a oxazolyl radical, the radical —CO—NR31R32, or the radical —CS—NR31R32,
   where
   R31 is 1-4C-alkyl, 3-7C-cycloalkyl, 1-4C-alkylsulfonyl, aryl, 1-4C-alkoxy,
   R32 is hydrogen or 1-4C-alkyl
   or where
   R31 and R32 together and including the nitrogen atom to which they are attached form a aziridino, azetidino, hydroxyazetidino, or piperazino radical,
   where aryl is phenyl or phenyl substituted with 1-4C-alkoxy,
Arom is phenyl,
with the proviso that
when
R2 is 1-4C-alkyl
then
R3 is cyano, a oxazolyl radical, the radical —CO—NR31R32, or the radical CS—NR31R32,
   where for —CO—NR31R32
   R31 is 3-7C-cycloalkyl, 1-4C-alkylsulfonyl, aryl, 1-4C-alkoxy,
   R32 is hydrogen or 1-4C-alkyl
   and for —CS—NR31R32
   R31 is 1-4C-alkyl
   R32 is 1-4C-alkyl
   or where
   R31 and R32 together and including the nitrogen atom to which they are attached form a aziridino, azetidino, hydroxyazetidino, or piperazino radical,
      and their salts.

Particular emphasis is also given to compounds of the formula 1, where
R1 is 1-4C-alkyl,
R2 is hydroxy-3-4C-alkinyl, carboxyl, mono- or di-1-4C-alkylamino-1-4C-alkyl, 1-4C-alkylcarbonyl, 2-4C-alkinylcarbonyl or the radical —CO—NR21R22,
   where
   R21 is 1-4C-alkyl or 1-4C-alkoxy-1-4C-alkyl and
   R22 is hydrogen or 1-4C-alkyl
R3 is the radical —CO—NR31R32,
   where
   R31 is 1-4C-alkyl,
   R32 is 1-4C-alkyl,
Arom is phenyl,
   and their salts.

Particular emphasis is also given to compounds of the formula 1, where
R1 is 1-4C-alkyl,
R2 is 1-4C-alkyl,
R3 is cyano, a oxazolyl radical, the radical —CO—NR31R32, or the radical —CS—NR31R32,
   where for —CO—NR31R32
   R31 is 3-7C-cycloalkyl, 1-4C-alkylsulfonyl, aryl, 1-4C-alkoxy,
   R32 is hydrogen, 1-4C-alkyl
   and for —CS—NR31R32
   R31 is 1-4C-alkyl
   R32 is 1-4C-alkyl
   or where
   R31 and R32 together and including the nitrogen atom to which they are attached form a aziridino, azetidino, hydroxyazetidino, or piperazino radical,
   where aryl is phenyl or phenyl substituted with 1-4C-alkoxy,
Arom is phenyl,
   and their salts.

Particular emphasis is also given to compounds of the formula 1, where
R1 is 1-4C-alkyl,
R2 is 1-4C-alkyl, hydroxy-3-4C-alkinyl, carboxyl, mono- or di-1-4C-alkylamino-1-4C-alkyl, 1-4C-alkylcarbonyl, 2-4C-alkinylcarbonyl or the radical —CO—NR21R22,
   where
   R21 is hydrogen, 1-4C-alkyl or 1-4C-alkoxy-1-4C-alkyl and
   R22 is hydrogen, 1-4C-alkyl or 1-4C-alkoxy-1-4C-alkyl,
R3 is a oxazolyl radical or the radical —CO—NR31R32,
   where
   R31 is 1-4C-alkyl or 3-7C-cycloalkyl
   R32 is hydrogen or 1-4C-alkyl,
   or where
   R31 and R32 together and including the nitrogen atom to which they are attached form a aziridino or azetidino radical,
Arom is phenyl,
with the proviso that
when
R2 is 1-4C-alkyl
then
R3 is a oxazolyl radical or the radical —CO—NR31R32,
   where
   R31 is 3-7C-cycloalkyl
   R32 is hydrogen
   or where
   R31 and R32 together and including the nitrogen atom to which they are attached form a aziridino or azetidino radical,
      and their salts.

Particular emphasis is also given to compounds of the formula 1, where
R1 is 1-4C-alkyl,
R2 is hydroxy-3-4C-alkinyl, carboxyl, mono- or di-1-4C-alkylamino-1-4C-alkyl, 1-4C-alkylcarbonyl, 2-4C-alkinylcarbonyl or the radical —CO—NR21R22,
   where
   R21 is hydrogen, 1-4C-alkyl or 1-4C-alkoxy-1-4C-alkyl and
   R22 is hydrogen, 1-4C-alkyl or 1-4C-alkoxy-1-4C-alkyl,
R3 is the radical —CO—NR31R32,
   where
   R31 is 1-4C-alkyl,
   R32 is 1-4C-alkyl,
Arom is phenyl,
   and their salts.

Particular emphasis is also given to compounds of the formula 1, where
R1 is 1-4C-alkyl,
R2 is 1-4C-alkyl,
R3 is a oxazolyl radical or the radical —CO—NR31R32,
   where
   R31 is 3-7C-cycloalkyl
   R32 is hydrogen,
   or where
   R31 and R32 together and including the nitrogen atom to which they are attached form a aziridino or azetidino radical,
Arom is phenyl,
   and their salts.

Particular emphasis is also given to compounds of the formula 1, where
R1 is 1-4C-alkyl
R2 is carboxyl, mono- or di-1-4C-alkylamino-1-4C-alkyl or the radical —CO—NR21R22,
   where
   R21 is hydrogen, 1-4C-alkyl or 1-4C-alkoxy-1-4C-alkyl and
   R22 is hydrogen, 1-4C-alkyl or 1-4C-alkoxy-1-4C-alkyl,
R3 is the radical —CO—NR31R32,
   where
   R31 is 1-4C-alkyl and
   R32 is 1-4C-alkyl
Arom is Phenyl
   and their salts.

Among the compounds of the formula 1 according to the invention including the compounds according to the aspects a to h, and those to be mentioned, particularly mentioned and to which emphasis and particular emphasis is given, the optically pure compounds of the formula 1-a are preferred

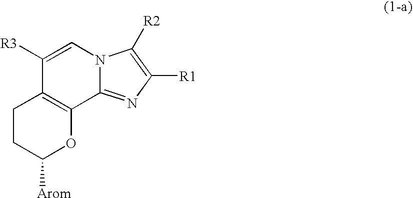

The invention also relates to compounds of the formula 1a,

where

R1 is hydrogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl or hydroxy-1-4C-alkyl,
R2 is hydrogen, 1-4C-alkyl, 3-7C-cycloalkyl, 3-7C-cycloalkyl-1-4C-alkyl, 1-4C-alkoxycarbonyl, hydroxy-1-4C-alkyl, hydroxy-3-4C-alkenyl, hydroxy-3-4C-alkinyl, halogen, 2-4C-alkenyl, 2-4C-alkynyl, fluoro-1-4C-alkyl, cyanomethyl, hydroxy, 1-4C-alkoxy, amino, mono- or di-1-4C-alkylamino, 1-4C-alkylcarbonylamino, 1-4C-alkoxycarbonylamino, 1-4C-alkoxy-1-4C-alkoxycarbonylamino, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR21R22,
   where
   R21 is hydrogen, 1-7C-alkyl, hydroxy-1-4C-alkyl, 1-4C-alkoxy-1-4C-alkyl or 3-7C-cycloalkyl and
   R22 is hydrogen, 1-7C-alkyl, hydroxy-1-4C-alkyl, 1-4C-alkoxy-1-4C-alkyl or 3-7C-cycloalkyl,
   or where
   R21 and R22 together and including the nitrogen atom to which they are attached form a pyrrolidino, piperidino, morpholino, aziridino or azetidino radical,
R3 is hydroxy-1-4C-alkyl, 1-4C-alkoxy-1-4C-alkyl, 1-4C-alkoxy-1-4C-alkoxy-1-4C-alkyl, 1-4C-alkoxycarbonyl, fluoro-1-4C-alkoxy-1-4C-alkyl, a imidazolyl, tetrazolyl or oxazolyl radical or the radical —CO—NR31R32,
   where
   R31 is hydrogen, 1-7C-alkyl, hydroxy-1-4C-alkyl, 1-4C-alkoxy-1-4C-alkyl or 3-7C-cycloalkyl and
   R32 is hydrogen, 1-7C-alkyl, hydroxy-1-4C-alkyl, 1-4C-alkoxy-1-4C-alkyl or 3-7C-cycloalkyl,
   or where
   R31 and R32 together and including the nitrogen atom to which they are attached form a pyrrolidino, piperidino, morpholino, aziridino or azetidino radical,
Arom is a R4-, R5-, R6- and R7-substituted mono- or bicyclic aromatic radical selected from the group consisting of phenyl, naphthyl, pyrrolyl, pyrazolyl, imidazolyl, 1,2,3-triazolyl, indolyl, benzimidazolyl, furanyl (furyl), benzofuranyl (benzofuryl), thiophenyl (thienyl), benzothiophenyl (benzothienyl), thiazolyl, isoxazolyl, pyridinyl, pyrimidinyl, quinolinyl and isoquinolinyl,
   where
   R4 is hydrogen, 1-4C-alkyl, hydroxy-1-4C-alkyl, 1-4C-alkoxy, 2-4C-alkenyloxy, 1-4C-alkylcarbonyl, carboxyl, 1-4C-alkoxycarbonyl, carboxy-1-4C-alkyl, 1-4C-alkoxycarbonyl-1-4C-alkyl, halogen, hydroxyl, aryl, aryl-1-4C-alkyl, aryloxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R5 is hydrogen, 1-4C-alkyl, 1-4C-alkoxy, 1-4C-alkoxycarbonyl, halogen, trifluoromethyl or hydroxyl,
   R6 is hydrogen, 1-4C-alkyl or halogen and
   R7 is hydrogen, 1-4C-alkyl or halogen,
   where
   aryl is phenyl or substituted phenyl having one, two or three identical or different substitutents from the group consisting of 1-4C-alkyl, 1-4C-alkoxy, carboxyl, 1-4C-alkoxycarbonyl, halogen, trifluoromethyl, nitro, trifluoromethoxy, hydroxyl and cyano,
with the proviso that,
when
R2 is hydrogen, 1-4C-alkyl, 3-7C-cycloalkyl, 3-7C-cycloalkyl-1-4C-alkyl, 1-4C-alkoxycarbonyl, hydroxy-1-4C-alkyl, halogen, 2-4C-alkenyl, 2-4C-alkynyl, fluoro-1-4C-alkyl or cyanomethyl,
then
R3 is a imidazolyl, tetrazolyl or oxazolyl radical or the radical —CO—NR31R32,
   where
   R31 is 3-7C-cycloalkyl and
   R32 is hydrogen, 1-7C-alkyl, hydroxy-1-4C-alkyl, 1-4C-alkoxy-1-4C-alkyl or 3-7C-cycloalkyl,
   or where R31 and R32 together and including the nitrogen atom to which they are attached form a aziridino or azetidino radical,
      and their salts.

Compounds of the formula 1a which are to be mentioned are those, where
R1 is hydrogen, 1-4C-alkyl, 3-7C-cycloalkyl
R2 is hydrogen, 1-4C-alkyl, 3-7C-cycloalkyl, 3-7C-cycloalkyl-1-4C-alkyl, 1-4C-alkoxycarbonyl, hydroxy-1-4C-alkyl, hydroxy-3-4C-alkenyl, hydroxy-3-4C-alkinyl, halogen, 2-4C-alkenyl, 2-4C-alkynyl, fluoro-1-4C-alkyl, cyanomethyl, hydroxy, 1-4C-alkoxy, amino, mono- or di-1-4C-alkylamino, 1-4C-alkylcarbonylamino, 1-4C-alkoxycarbonylamino, 1-4C-alkoxy-1-4C-alkoxycarbonylamino, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR21R22,
   where
   R21 is hydrogen, 1-7C-alkyl, hydroxy-1-4C-alkyl, 1-4C-alkoxy-1-4C-alkyl or 3-7C-cycloalkyl and
   R22 is hydrogen, 1-7C-alkyl, hydroxy-1-4C-alkyl, 1-4C-alkoxy-1-4C-alkyl or 3-7C-cycloalkyl,
   or where
   R21 and R22 together and including the nitrogen atom to which they are attached form a pyrrolidino, piperidino, morpholino, aziridino or azetidino radical,
R3 is hydroxy-1-4C-alkyl, 1-4C-alkoxy-1-4C-alkyl, 1-4C-alkoxy-1-4C-alkoxy-1-4C-alkyl, 1-4C-alkoxycarbonyl, fluoro-1-4C-alkoxy-1-4C-alkyl, a imidazolyl, tetrazolyl or oxazolyl radical or the radical —CO—NR31R32,
   where
   R31 is hydrogen, 1-7C-alkyl, hydroxy-1-4C-alkyl, 1-4C-alkoxy-1-4C-alkyl or 3-7C-cycloalkyl and
   R32 is hydrogen, 1-7C-alkyl, hydroxy-1-4C-alkyl, 1-4C-alkoxy-1-4C-alkyl or 3-7C-cycloalkyl,
   or where
   R31 and R32 together and including the nitrogen atom to which they are attached form a pyrrolidino, piperidino, morpholino, aziridino or azetidino radical,
Arom is a R4-, R5-, R6- and R7-substituted phenyl
   where
   R4 is hydrogen or 1-4C-alkyl, halogen, 1-4C-alkoxy, trifluoromethyl
   R5 is hydrogen or 1-4C-alkyl, halogen
   R6 is hydrogen and
   R7 is hydrogen
with the proviso that,
when
R2 is hydrogen, 1-4C-alkyl, 3-7C-cycloalkyl, 3-7C-cycloalkyl-1-4C-alkyl, 1-4C-alkoxycarbonyl, hydroxy-1-4C-alkyl, halogen, 2-4C-alkenyl, 2-4C-alkynyl, fluoro-1-4C-alkyl or cyanomethyl,
then
R3 is a imidazolyl, tetrazolyl or oxazolyl radical or the radical —CO—NR31R32,
   where
   R31 is 3-7C-cycloalkyl and
   R32 is hydrogen, 1-7C-alkyl, hydroxy-1-4C-alkyl, 1-4C-alkoxy-1-4C-alkyl or 3-7C-cycloalkyl,
   or where R31 and R32 together and including the nitrogen atom to which they are attached form a aziridino or azetidino radical,
      and their salts.

Particular mention may be made of those compounds of the formula 1a, where
R1 is hydrogen, 1-4C-alkyl or 3-7C-cycloalkyl,
R2 is hydrogen, 1-4C-alkyl, hydroxy-3-4C-alkenyl, hydroxy-3-4C-alkinyl, hydroxy, 1-4C-alkoxy, amino, mono- or di-1-4C-alkylamino, 1-4C-alkylcarbonylamino, 1-4C-alkoxycarbonylamino, 1-4C-alkoxy-1-4C-alkoxycarbonylamino, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR21R22,
   where
   R21 is hydrogen, 1-7C-alkyl, hydroxy-1-4C-alkyl, 1-4C-alkoxy-1-4C-alkyl or 3-7C-cycloalkyl and
   R22 is hydrogen, 1-7C-alkyl, hydroxy-1-4C-alkyl, 1-4C-alkoxy-1-4C-alkyl or 3-7C-cycloalkyl,
   or where
   R21 and R22 together and including the nitrogen atom to which they are attached form a pyrrolidino, piperidino, morpholino, aziridino or azetidino radical,
R3 is hydroxy-1-4C-alkyl, 1-4C-alkoxy-1-4C-alkyl, 1-4C-alkoxy-1-4C-alkoxy-1-4C-alkyl, 1-4C-alkoxycarbonyl, fluoro-1-4C-alkoxy-1-4C-alkyl, a imidazolyl, tetrazolyl or oxazolyl radical or the radical —CO—NR31R32,
   where
   R31 is hydrogen, 1-7C-alkyl, hydroxy-1-4C-alkyl, 1-4C-alkoxy-1-4C-alkyl or 3-7C-cycloalkyl and
   R32 is hydrogen, 1-7C-alkyl, hydroxy-1-4C-alkyl, 1-4C-alkoxy-1-4C-alkyl or 3-7C-cycloalkyl,
   or where
   R31 and R32 together and including the nitrogen atom to which they are attached form a pyrrolidino, piperidino, morpholino, aziridino or azetidino radical,
Arom is a R4-, R5-, R6- and R7-substituted phenyl
   where
   R4 is hydrogen or 1-4C-alkyl, halogen, 1-4C-alkoxy, trifluoromethyl
   R5 is hydrogen or 1-4C-alkyl, halogen
   R6 is hydrogen and
   R7 is hydrogen
with the proviso that,
when
R2 is hydrogen or 1-4C-alkyl,
then
R3 is a imidazolyl, tetrazolyl or oxazolyl radical or the radical —CO—NR31R32,
   where
   R31 is 3-7C-cycloalkyl and
   R32 is hydrogen, 1-7C-alkyl, hydroxy-1-4C-alkyl, 1-4C-alkoxy-1-4C-alkyl or 3-7C-cycloalkyl,
   or where R31 and R32 together and including the nitrogen atom to which they are attached form a aziridino or azetidino radical,
      and their salts.

Emphasis is given to compounds of the formula 1a, where
R1 is 1-4C-alkyl,
R2 is hydroxy-3-4C-alkenyl, hydroxy-3-4C-alkinyl, hydroxy, 1-4C-alkoxy, amino, mono- or di-1-4C-alkylamino, 1-4C-alkylcarbonylamino, 1-4C-alkoxycarbonylamino, 1-4C-alkoxy-1-4C-alkoxycarbonylamino, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR21R22,
   where
   R21 is hydrogen, 1-4C-alkyl, hydroxy-1-4C-alkyl, 1-4C-alkoxy-1-4C-alkyl or 3-7C-cycloalkyl and
   R22 is hydrogen, 1-4C-alkyl, hydroxy-1-4C-alkyl, 1-4C-alkoxy-1-4C-alkyl or 3-7C-cycloalkyl,
   or where
   R21 and R22 together and including the nitrogen atom to which they are attached form a pyrrolidino, piperidino, morpholino, aziridino or azetidino radical,
R3 is a imidazolyl, tetrazolyl or oxazolyl radical or the radical —CO—NR31R32,
   where
   R31 is hydrogen, 1-4C-alkyl or 3-7C-cycloalkyl
   R32 is hydrogen, 1-4C-alkyl or 3-7C-cycloalkyl,
   or where
   R31 and R32 together and including the nitrogen atom to which they are attached form a pyrrolidino, piperidino, morpholino, aziridino or azetidino radical,
Arom is phenyl
   and their salts.

Emphasis is also given to compounds of the formula 1a, where
R1 is 1-4C-alkyl
R2 is hydroxy-3-4C-alkenyl, hydroxy-3-4C-alkinyl, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR21R22,
   where
   R21 is hydrogen, 1-4C-alkyl or 1-4C-alkoxy-1-4C-alkyl and
   R22 is hydrogen, 1-4C-alkyl or 1-4C-alkoxy-1-4C-alkyl,
R3 is the radical —CO—NR31R32,
   where
   R31 is 1-4C-alkyl and
   R32 is 1-4C-alkyl
Arom is phenyl
   and their salts.

Emphasis is also given to compounds of the formula 1a, where
R1 is 1-4C-alkyl,
R2 is 1-4C-alkyl,
R3 is a imidazolyl, tetrazolyl or oxazolyl radical or the radical —CO—NR31R32,
   where
   R31 is 3-7C-cycloalkyl
   R32 is hydrogen, 1-4C-alkyl or 3-7C-cycloalkyl,
   or where
   R31 and R32 together and including the nitrogen atom to which they are attached form a pyrrolidino, piperidino, morpholino, aziridino or azetidino radical,
Arom is phenyl,
   and their salts.

Compounds of the formula 1a which are also to be mentioned are those, where
R1 is hydrogen, 1-4C-alkyl, 3-7C-cycloalkyl
R2 is hydrogen, 1-4C-alkyl, 3-7C-cycloalkyl, 3-7C-cycloalkyl-1-4C-alkyl, 1-4C-alkoxycarbonyl, hydroxy-1-4C-alkyl, hydroxy-3-4C-alkenyl, hydroxy-3-4C-alkinyl, halogen, 2-4C-alkenyl, 2-4C-alkynyl, fluoro-1-4C-alkyl, cyanomethyl, hydroxy, 1-4C-alkoxy, amino, mono- or di-1-4C-alkylamino, 1-4C-alkylcarbonylamino, 1-4C-alkoxycarbonylamino, 1-4C-alkoxy-1-4C-alkoxycarbonylamino, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR21R22,
   where
   R21 is hydrogen, 1-7C-alkyl, hydroxy-1-4C-alkyl, 1-4C-alkoxy-1-4C-alkyl or 3-7C-cycloalkyl and
   R22 is hydrogen, 1-7C-alkyl, hydroxy-1-4C-alkyl, 1-4C-alkoxy-1-4C-alkyl or 3-7C-cycloalkyl,
   or where
   R21 and R22 together and including the nitrogen atom to which they are attached form a pyrrolidino, piperidino, morpholino, aziridino or azetidino radical,
R3 is 1-4C-alkylcarbonyl, hydroxy-1-4C-alkyl, 1-4C-alkoxy-1-4C-alkyl, 1-4C-alkoxy-1-4C-alkoxy-1-4C-alkyl, 1-4C-alkoxycarbonyl, fluoro-1-4C-alkoxy-1-4C-alkyl, cyano, the radical —CO—NR31R32, the radical SO₂—NR31R32, the radical —CS—NR31R32, the radical C═N(OH)—NR31R32 or the group Het
   where
   R31 is hydrogen, amino, 1-7C-alkyl, hydroxy, hydroxy-1-4C-alkyl, 1-4C-alkoxy, 1-4C-alkoxy-1-4C-alkyl or 3-7C-cycloalkyl, 1-4C-alkylsulfonyl, arylsulfonyl, aryl-1-4C-alkylsulfonyl, aryl and
   R32 is hydrogen, 1-7C-alkyl, hydroxy-1-4C-alkyl, 1-4C-alkoxy-1-4C-alkyl or 3-7C-cycloalkyl,
   or where
   R31 and R32 together and including the nitrogen atom to which they are attached form a cyclic residue, substituted by R33, R34 and R35, selected from the group consisting of pyrrolidino, piperidino, piperazino, morpholino, aziridino or azetidino, and
   Het is a heterocyclic residue, substituted by R33, R34 and R35, selected from the group consisting of oxadiazol, dihydrooxazol, dihydroimidazol, oxazol, imidazol, isoxazol, dihydroisoxazol, pyrazol, and tetrazol
   where
   R33 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R34 is hydrogen, 1-4C-alkyl, 1-4C-alkoxy, 1-4C-alkoxycarbonyl, halogen, trifluormethyl or hydroxy,
   R35 is hydrogen, 1-4C-alkyl, 1-4C-alkoxy, 1-4C-alkoxycarbonyl, halogen, trifluormethyl or hydroxy,
   where
   aryl is phenyl or substituted phenyl having one, two or three identical or different substitutents from the group consisting of 1-4C-alkyl, 1-4C-alkoxy, carboxyl, 1-4C-alkoxycarbonyl, halogen, trifluoromethyl, nitro, trifluoromethoxy, hydroxy and cyano,
Arom is a R4- and R5-substituted phenyl, pyrrolyl, furanyl (furyl), thiophenyl (thienyl)
   where
   R4 is hydrogen or 1-4C-alkyl, halogen, 1-4C-alkoxy, trifluoromethyl
   R5 is hydrogen or 1-4C-alkyl, halogen
with the proviso that,
when
R2 is hydrogen, 1-4C-alkyl, 3-7C-cycloalkyl, 3-7C-cycloalkyl-1-4C-alkyl, 1-4C-alkoxycarbonyl, hydroxy-1-4C-alkyl, halogen, 2-4C-alkenyl, 2-4C-alkynyl, fluoro-1-4C-alkyl or cyanomethyl,
then
R3 is 1-4C-alkylcarbonyl, cyano, the radical —CO—NR31R32, the radical —SO₂—NR31R32, the radical —CS—NR31R32, the radical C═N(OH)—NR31R32 or the group Het
   where for the radical CO—NR31R32
   R31 is amino, hydroxy, 1-4C-alkoxy, 3-7C-cycloalkyl, 1-4C-alkylsulfonyl, arylsulfonyl, aryl-1-4C-alkylsulfonyl, aryl and
   R32 is hydrogen, 1-7C-alkyl, hydroxy-1-4C-alkyl, 1-4C-alkoxy-1-4C-alkyl or 3-7C-cycloalkyl, and for the radicals —SO₂—NR31R32, —CS—NR31R32, and C═N(OH)—NR31R32
   R31 is hydrogen, amino, 1-7C-alkyl, hydroxy, hydroxy-1-4C-alkyl, 1-4C-alkoxy, 1-4C-alkoxy-1-4C-alkyl or 3-7C-cycloalkyl, 1-4C-alkylsulfonyl, arylsulfonyl, aryl-1-4C-alkylsulfonyl, aryl and
   R32 is hydrogen, 1-7C-alkyl, hydroxy-1-4C-alkyl, 1-4C-alkoxy-1-4C-alkyl or 3-7C-cycloalkyl,
   or where
   R31 and R32 together and including the nitrogen atom to which they are attached form a cyclic residue, substituted by R33, R34 and R35, selected from the group consisting of pyrrolidino, piperidino, piperazino, morpholino, aziridino or azetidino where in the case of pyrrolidino, piperidino, or morpholino, at least one of the substitutents R33, R34, or R35 has to be different from hydrogen, and
   Het is a heterocyclic residue, substituted by R33, R34 and R35, selected from the group consisting of oxadiazol, dihydrooxazol, dihydroimidazol, oxazol, imidazol, isoxazol, dihydroisoxazol, pyrazol, and tetrazol
   where
   R33 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R34 is hydrogen, 1-4C-alkyl, 1-4C-alkoxy, 1-4C-alkoxycarbonyl, halogen, trifluormethyl or hydroxy,
   R35 is hydrogen, 1-4C-alkyl, 1-4C-alkoxy, 1-4C-alkoxycarbonyl, halogen, trifluormethyl or hydroxy,
   where
   aryl is phenyl or substituted phenyl having one, two or three identical or different substitutents from the group consisting of 1-4C-alkyl, 1-4C-alkoxy, carboxyl, 1-4C-alkoxycarbonyl, halogen, trifluoromethyl, nitro, trifluoromethoxy, hydroxy and cyano,
      and their salts.

Particular mention may also be made of those compounds of the formula 1a, where
R1 is hydrogen, 1-4C-alkyl or 3-7C-cycloalkyl,
R2 is hydrogen, 1-4C-alkyl, hydroxy-3-4C-alkenyl, hydroxy-3-4C-alkinyl, hydroxy, 1-4C-alkoxy, amino, mono- or di-1-4C-alkylamino, 1-4C-alkylcarbonylamino, 1-4C-alkoxycarbonylamino, 1-4C-alkoxy-1-4C-alkoxycarbonylamino, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR21R22,
   where
   R21 is hydrogen, 1-7C-alkyl, hydroxy-1-4C-alkyl, 1-4C-alkoxy-1-4C-alkyl or 3-7C-cycloalkyl and
   R22 is hydrogen, 1-7C-alkyl, hydroxy-1-4C-alkyl, 1-4C-alkoxy-1-4C-alkyl or 3-7C-cycloalkyl,
   or where
   R21 and R22 together and including the nitrogen atom to which they are attached form a pyrrolidino, piperidino, morpholino, aziridino or azetidino radical,
R3 is 1-4C-alkylcarbonyl, hydroxy-1-4C-alkyl, 1-4C-alkoxy-1-4C-alkyl, 1-4C-alkoxy-1-4C-alkoxy-1-4C-alkyl, 1-4C-alkoxycarbonyl, fluoro-1-4C-alkoxy-1-4C-alkyl, cyano, the radical —CO—NR31R32, the radical —SO₂—NR31R32, the radical —CS—NR31R32, the radical C═N(OH)—NR31R32 or the group Het
   where
   R31 is hydrogen, amino, 1-7C-alkyl, hydroxy, hydroxy-1-4C-alkyl, 1-4C-alkoxy, 1-4C-alkoxy-1-4C-alkyl, 3-7C-cycloalkyl, 1-4C-alkylsulfonyl, arylsulfonyl, aryl-1-4C-alkylsulfonyl or aryl and
   R32 is hydrogen, 1-7C-alkyl, hydroxy-1-4C-alkyl, 1-4C-alkoxy-1-4C-alkyl or 3-7C-cycloalkyl,
   or where
   R31 and R32 together and including the nitrogen atom to which they are attached form a cyclic residue, substituted by R33, R34 and R35, selected from the group consisting of pyrrolidino, piperidino, piperazino, morpholino, aziridino or azetidino, and
   Het is a heterocyclic residue, substituted by R33, R34 and R35, selected from the group consisting of oxadiazol, dihydrooxazol, dihydroimidazol, oxazol, imidazol, isoxazol, dihydroisoxazol, pyrazol, and tetrazol
   where
   R33 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R34 is hydrogen, 1-4C-alkyl, 1-4C-alkoxy, 1-4C-alkoxycarbonyl, halogen, trifluormethyl or hydroxy,
   R35 is hydrogen, 1-4C-alkyl, 1-4C-alkoxy, 1-4C-alkoxycarbonyl, halogen, trifluormethyl or hydroxy,
   where
   aryl is phenyl or substituted phenyl having one, two or three identical or different substitutents from the group consisting of 1-4C-alkyl, 1-4C-alkoxy, carboxyl, 1-4C-alkoxycarbonyl, halogen, trifluoromethyl, nitro, trifluoromethoxy, hydroxy and cyano,
Arom is a R4- and R5-substituted phenyl, pyrrolyl, furanyl (furyl), thiophenyl (thienyl)
   where
   R4 is hydrogen or 1-4C-alkyl, halogen, 1-4C-alkoxy, trifluoromethyl
   R5 is hydrogen or 1-4C-alkyl, halogen
with the proviso that,
when
R2 is hydrogen or 1-4C-alkyl,
then
R3 is 1-4C-alkylcarbonyl, cyano, the radical —CO—NR31R32, the radical —SO₂—NR31R32, the radical —CS—NR31R32, the radical C═N(OH)—NR31R32 or the group Het
   where for the radical —CO—NR31R32
   R31 is amino, hydroxy, 1-4C-alkoxy, 3-7C-cycloalkyl, 1-4C-alkylsulfonyl, arylsulfonyl, aryl-1-4C-alkylsulfonyl, aryl and
   R32 is hydrogen, 1-7C-alkyl, hydroxy-1-4C-alkyl, 1-4C-alkoxy-1-4C-alkyl or 3-7C-cycloalkyl, and for the radicals —SO₂—NR31R32, —CS—NR31R32, and C═N(OH)—NR31R32
   R31 is hydrogen, amino, 1-7C-alkyl, hydroxy, hydroxy-1-4C-alkyl, 1-4C-alkoxy, 1-4C-alkoxy-1-4C-alkyl, 3-7C-cycloalkyl, 1-4C-alkylsulfonyl, arylsulfonyl, aryl-1-4C-alkylsulfonyl or aryl and
   R32 is hydrogen, 1-7C-alkyl, hydroxy-1-4C-alkyl, 1-4C-alkoxy-1-4C-alkyl or 3-7C-cycloalkyl,
   or where
   R31 and R32 together and including the nitrogen atom to which they are attached form a cyclic residue, substituted by R33, R34 and R35, selected from the group consisting of pyrrolidino, piperidino, piperazino, morpholino, aziridino or azetidino where in the case of pyrrolidino, piperidino, or morpholino, at least one of the substitutents R33, R34, or R35 has to be different from hydrogen, and
   Het is a heterocyclic residue, substituted by R33, R34 and R35, selected from the group consisting of oxadiazol, dihydrooxazol, dihydroimidazol, oxazol, imidazol, isoxazol, dihydroisoxazol, pyrazol, and tetrazol
   where
   R33 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R34 is hydrogen, 1-4C-alkyl, 1-4C-alkoxy, 1-4C-alkoxycarbonyl, halogen, trifluormethyl or hydroxy,
   R35 is hydrogen, 1-4C-alkyl, 1-4C-alkoxy, 1-4C-alkoxycarbonyl, halogen, trifluormethyl or hydroxy,
   where
   aryl is phenyl or substituted phenyl having one, two or three identical or different substitutents from the group consisting of 1-4C-alkyl, 1-4C-alkoxy, carboxyl, 1-4C-alkoxycarbonyl, halogen, trifluoromethyl, nitro, trifluoromethoxy, hydroxy and cyano,
      and their salts.

Emphasis is also given to compounds of the formula 1a, where
R1 is 1-4C-alkyl,
R2 is 1-4C-alkyl, hydroxy-3-4C-alkenyl, hydroxy-3-4C-alkinyl, hydroxy, 1-4C-alkoxy, amino, mono- or di-1-4C-alkylamino, 1-4C-alkylcarbonylamino, 1-4C-alkoxycarbonylamino, 1-4C-alkoxy-1-4C-alkoxycarbonylamino, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR21R22,
   where
   R21 is hydrogen, 1-4C-alkyl, hydroxy-1-4C-alkyl, 1-4C-alkoxy-1-4C-alkyl or 3-7C-cycloalkyl and
   R22 is hydrogen, 1-4C-alkyl, hydroxy-1-4C-alkyl, 1-4C-alkoxy-1-4C-alkyl or 3-7C-cycloalkyl,
   or where
   R21 and R22 together and including the nitrogen atom to which they are attached form a pyrrolidino, piperidino, morpholino, aziridino or azetidino radical,
R3 is cyano, the radical —CO—NR31R32, the radical —CS—NR31R32, or the group Het
   where
   R31 is hydrogen, 1-7C-alkyl, 1-4C-alkoxy, 3-7C-cycloalkyl, 1-4C-alkylsulfonyl, arylsulfonyl, aryl-1-4C-alkylsulfonyl, aryl and
   R32 is hydrogen, 1-7C-alkyl, or 3-7C-cycloalkyl,
   or where
   R31 and R32 together and including the nitrogen atom to which they are attached form a cyclic residue, substituted by R33, selected from the group consisting of pyrrolidino, piperidino, piperazino, morpholino, aziridino or azetidino, and
   Het is a heterocyclic residue, substituted by R33, selected from the group consisting of oxadiazol, dihydrooxazol, dihydroimidazol, oxazol, imidazol, isoxazol, dihydroisoxazol, pyrazol, and tetrazol
   where
   R33 is hydrogen, 1-4C-alkyl, 1-4C-alkoxy, 1-4C-alkylcarbonyl, 1-4C-alkoxycarbonyl, halogen, hydroxy
   where
   aryl is phenyl or substituted phenyl having one, two or three identical or different substitutents from the group consisting of 1-4C-alkyl, 1-4C-alkoxy, carboxyl, 1-4C-alkoxycarbonyl, halogen, trifluoromethyl, nitro, trifluoromethoxy, hydroxy and cyano,
Arom is a R4- and R5-substituted phenyl, pyrrolyl, furanyl (furyl), thiophenyl (thienyl)
   where
   R4 is hydrogen or 1-4C-alkyl, halogen, 1-4C-alkoxy, trifluoromethyl
   R5 is hydrogen or 1-4C-alkyl, halogen
with the proviso that,
when
R2 is 1-4C-alkyl,
then
R3 is cyano, the radical —CO—NR31R32, the radical —CS—NR31R32, or the group Het
   where for the radical —CO—NR31R32
   R31 is 1-4C-alkoxy, 3-7C-cycloalkyl, 1-4C-alkylsulfonyl, arylsulfonyl, aryl-1-4C-alkylsulfonyl, aryl and
   R32 is hydrogen, 1-7C-alkyl, or 3-7C-cycloalkyl,
   and for the radical —CS—NR31R32
   R31 is hydrogen, 1-7C-alkyl, 1-4C-alkoxy, 3-7C-cycloalkyl, 1-4C-alkylsulfonyl, arylsulfonyl, aryl-1-4C-alkylsulfonyl, aryl and
   R32 is hydrogen, 1-7C-alkyl, or 3-7C-cycloalkyl,
   or where
   R31 and R32 together and including the nitrogen atom to which they are attached form a cyclic residue, substituted by R33, selected from the group consisting of pyrrolidino, piperidino, piperazino, morpholino, aziridino or azetidino where in the case of pyrrolidino, piperidino, or morpholino, the substitutent R33 has to be different from hydrogen, and
   Het is a heterocyclic residue, substituted by R33, selected from the group consisting of oxadiazol, dihydrooxazol, dihydroimidazol, oxazol, imidazol, isoxazol, dihydroisoxazol, pyrazol, and tetrazol
   where
   R33 is hydrogen, 1-4C-alkyl, 1-4C-alkoxy, 1-4C-alkylcarbonyl, 1-4C-alkoxycarbonyl, halogen, hydroxy,
   where
   aryl is phenyl or substituted phenyl having one, two or three identical or different substituents from the group consisting of 1-4C-alkyl, 1-4C-alkoxy, carboxyl, 1-4C-alkoxycarbonyl, halogen, trifluoromethyl, nitro, trifluoromethoxy, hydroxy and cyano,
      and their salts.

Emphasis is also given to compounds of the formula 1a, where
R1 is 1-4C-alkyl,
R2 is 1-4C-alkyl, hydroxy-3-4C-alkenyl, hydroxy-3-4C-alkinyl, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR21R22,
   where
   R21 is hydrogen, 1-4C-alkyl, 1-4C-alkoxy-1-4C-alkyl and
   R22 is hydrogen, 1-4C-alkyl, 1-4C-alkoxy-1-4C-alkyl,
R3 is cyano, the radical —CO—NR31R32, the radical CS—NR31R32, or the group Het
   where
   R31 is hydrogen, 1-7C-alkyl, 1-4C-alkoxy, 3-7C-cycloalkyl, 1-4C-alkylsulfonyl, arylsulfonyl, aryl-1-4C-alkylsulfonyl, aryl and
   R32 is hydrogen, 1-7C-alkyl, or 3-7C-cycloalkyl,
   or where
   R31 and R32 together and including the nitrogen atom to which they are attached form a cyclic residue, substituted by R33, selected from the group consisting of pyrrolidino, piperazino, aziridino or azetidino, and
   Het is a heterocyclic residue, substituted by R33, selected from the group consisting of dihydrooxazol, dihydroimidazol, oxazol, imidazol, isoxazol, dihydroisoxazol, and tetrazol
   where
   R33 is hydrogen, 1-4C-alkyl, 1-4C-alkoxy, 1-4C-alkylcarbonyl, 1-4C-alkoxycarbonyl, halogen, hydroxy
   where
   aryl is phenyl or substituted phenyl having one, two or three identical or different substitutents from the group consisting of 1-4C-alkyl, 1-4C-alkoxy, 1-4C-alkoxycarbonyl, halogen, hydroxy,
Arom is a R4-substituted phenyl, pyrrolyl, furanyl (furyl), thiophenyl (thienyl)
   where
   R4 is hydrogen or 1-4C-alkyl, halogen, 1-4C-alkoxy, trifluoromethyl
with the proviso that,
when
R2 is 1-4C-alkyl,
then
R3 is cyano, the radical —CO—NR31R32, the radical —CS—NR31R32, or the group Het
   where for —CO—NR31R32
   R31 is 1-4C-alkoxy, 3-7C-cycloalkyl, 1-4C-alkylsulfonyl, arylsulfonyl, aryl-1-4C-alkylsulfonyl, aryl and
   R32 is hydrogen, 1-7C-alkyl, or 3-7C-cycloalkyl,
   and for —CS—NR31R32
   R31 is hydrogen, 1-7C-alkyl, 1-4C-alkoxy, 3-7C-cycloalkyl, 1-4C-alkylsulfonyl, arylsulfonyl, aryl-1-4C-alkylsulfonyl, aryl and
   R32 is hydrogen, 1-7C-alkyl, or 3-7C-cycloalkyl,
   or where
   R31 and R32 together and including the nitrogen atom to which they are attached form a cyclic residue, substituted by R33, selected from the group consisting of pyrrolidino, piperazino, aziridino or azetidino where in the case of pyrrolidino, the substitutent R33 has to be different from hydrogen, and
   Het is a heterocyclic residue, substituted by R33, selected from the group consisting of dihydrooxazol, dihydroimidazol, oxazol, imidazol, isoxazol, dihydroisoxazol, and tetrazol
   where
   R33 is hydrogen, 1-4C-alkyl, 1-4C-alkoxy, 1-4C-alkylcarbonyl, 1-4C-alkoxycarbonyl, halogen, hydroxy
   where
   aryl is phenyl or substituted phenyl having one, two or three identical or different substitutents from the group consisting of 1-4C-alkyl, 1-4C-alkoxy, 1-4C-alkoxycarbonyl, halogen, hydroxy,
      and their salts.

Emphasis is also given to compounds of the formula 1a, where
R1 is 1-4C-alkyl
R2 is hydroxy-3-4C-alkenyl, hydroxy-3-4C-alkinyl, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR21R22,
   where
   R21 is hydrogen, 1-4C-alkyl or 1-4C-alkoxy-1-4C-alkyl and
   R22 is hydrogen, 1-4C-alkyl or 1-4C-alkoxy-1-4C-alkyl,
R3 is cyano, the radical —CO—NR31R32, the radical —CS—NR31R32, or the group Het
   where
   R31 is hydrogen, 1-7C-alkyl, 1-4C-alkoxy, 3-7C-cycloalkyl, 1-4C-alkylsulfonyl, arylsulfonyl, aryl-1-4C-alkylsulfonyl, aryl and
   R32 is hydrogen, 1-7C-alkyl, or 3-7C-cycloalkyl,
   or where
   R31 and R32 together and including the nitrogen atom to which they are attached form a cyclic residue, substituted by R33, selected from the group consisting of pyrrolidino, piperazino, aziridino or azetidino, and
   Het is a heterocyclic residue, substituted by R33, selected from the group consisting of dihydrooxazol, dihydroimidazol, oxazol, imidazol, isoxazol, dihydroisoxazol, and tetrazol
   where
   R33 is hydrogen, 1-4C-alkyl, 1-4C-alkoxy, 1-4C-alkylcarbonyl, 1-4C-alkoxycarbonyl, halogen, hydroxy
   where
   aryl is phenyl or substituted phenyl having one, two or three identical or different substitutents from the group consisting of 1-4C-alkyl, 1-4C-alkoxy, 1-4C-alkoxycarbonyl, halogen, hydroxy,
Arom is a R4-substituted phenyl, pyrrolyl, furanyl (furyl), thiophenyl (thienyl)
   where
   R4 is hydrogen or 1-4C-alkyl, halogen, 1-4C-alkoxy, trifluoromethyl
      and their salts.

Emphasis is also given to compounds of the formula 1a, where
R1 is 1-4C-alkyl,
R2 is 1-4C-alkyl,
R3 is cyano, the radical —CO—NR31R32, the radical CS—NR31R32, or the group Het
   where for the radical —CO—NR31R32
   R31 1-4-C-alkoxy, 3-7C-cycloalkyl, 1-4C-alkylsulfonyl, arylsulfonyl, aryl-1-4C-alkylsulfonyl, aryl and
   R32 is hydrogen, 1-7C-alkyl, or 3-7C-cycloalkyl,
   for the radical —CS—NR31R32
   R31 is hydrogen, 1-7C-alkyl, 1-4C-alkoxy, 3-7C-cycloalkyl, 1-4C-alkylsulfonyl, arylsulfonyl, aryl-1-4C-alkylsulfonyl, aryl and
   R32 is hydrogen, 1-7C-alkyl, or 3-7C-cycloalkyl,
   or where
   R31 and R32 together and including the nitrogen atom to which they are attached form a cyclic residue, substituted by R33, selected from the group consisting of pyrrolidino, piperazino, aziridino or azetidino where in the case of pyrrolidino, the substitutent R33 has to be different from hydrogen, and
   Het is a heterocyclic residue, substituted by R33, selected from the group consisting of dihydrooxazol, dihydroimidazol, oxazol, imidazol, isoxazol, dihydroisoxazol, and tetrazol
   where
   R33 is hydrogen, 1-4C-alkyl, 1-4C-alkoxy, 1-4C-alkylcarbonyl, 1-4C-alkoxycarbonyl,
   where
   aryl is phenyl or substituted phenyl having one, two or three identical or different substitutents from the group consisting of 1-4C-alkyl, 1-4C-alkoxy, 1-4C-alkoxycarbonyl, halogen, hydroxy,
Arom is a R4-substituted phenyl, pyrrolyl, furanyl (furyl), thiophenyl (thienyl)
   where
   R4 is hydrogen or 1-4C-alkyl, halogen, 1-4C-alkoxy, trifluoromethyl
      and their salts.

Particular emphasis is also given to compounds of the formula 1a, where
R1 is 1-4C-alkyl,
R2 is 1-4C-alkyl, hydroxy-3-4C-alkinyl, carboxyl, mono- or di-1-4C-alkylamino-1-4C-alkyl, 1-4C-alkylcarbonyl, 2-4C-alkinylcarbonyl or the radical —CO—NR21R22,
   where
   R21 is 1-4C-alkyl or 1-4C-alkoxy-1-4C-alkyl and
   R22 is hydrogen or 1-4C-alkyl,
R3 is cyano, a oxazolyl radical, the radical —CO—NR31R32, or the radical —CS—NR31R32,
   where
   R31 is 1-4C-alkyl, 3-7C-cycloalkyl, 1-4C-alkylsulfonyl, aryl, 1-4C-alkoxy,
   R32 is hydrogen or 1-4C-alkyl
   or where
   R31 and R32 together and including the nitrogen atom to which they are attached form a aziridino, azetidino, hydroxyazetidino, or piperazino radical,
   where aryl, is phenyl or phenyl substituted with 1-4C-alkoxy,
Arom is phenyl,
with the proviso that
when
R2 is 1-4C-alkyl
then
R3 is cyano, a oxazolyl radical, the radical —CO—NR31R32, or the radical —CS—NR31R32,
   where for —CO—NR31R32
   R31 is 3-7C-cycloalkyl, 1-4C-alkylsulfonyl, aryl, 11-4C-alkoxy,
   R32 is hydrogen or 1-4C-alkyl
   and for —CS—NR31R32
   R31 is 1-4C-alkyl
   R32 is 1-4C-alkyl
   or where
   R31 and R32 together and including the nitrogen atom to which they are attached form a aziridino, azetidino, hydroxyazetidino, or piperazino radical,
      and their salts.

Particular emphasis is also given to compounds of the formula 1a, where
R1 is 1-4C-alkyl,
R2 is hydroxy-3-4C-alkinyl, carboxyl, mono- or di-1-4C-alkylamino-1-4C-alkyl, 1-4C-alkylcarbonyl, 2-4C-alkinylcarbonyl or the radical —CO—NR21R22,
   where
   R21 is 1-4C-alkyl or 1-4C-alkoxy-1-4C-alkyl and
   R22 is hydrogen or 1-4C-alkyl
R3 is the radical —CO—NR31R32,
   where
   R31 is 1-4C-alkyl,
   R32 is 1-4C-alkyl,
Arom is phenyl,
   and their salts.

Particular emphasis is also given to compounds of the formula 1a, where
R1 is 1-4C-alkyl,
R2 is 1-4C-alkyl,
R3 is cyano, a oxazolyl radical, the radical —CO—NR31R32, or the radical CS—NR31R32,
   where for —CO—NR31R32
   R31 is 3-7C-cycloalkyl, 1-4C-alkylsulfonyl, aryl, 1-4C-alkoxy,
   R32 is hydrogen, 1-4C-alkyl
   and for —CS—NR31R32
   R31 is 1-4C-alkyl
   R32 is 1-4C-alkyl
   or where
   R31 and R32 together and including the nitrogen atom to which they are attached form a aziridino, azetidino, hydroxyazetidino, or piperazino radical,
   where aryl is phenyl or phenyl substituted with 1-4C-alkoxy,
Arom is phenyl,
   and their salts.

Particular emphasis is also given to compounds of the formula 1a, where
R1 is 1-4C-alkyl,
R2 is 1-4C-alkyl or 1-4C-alkylcarbonyl,
R3 is the radical —CO—NR31R32 or the radical CS—NR31R32,
   where
   R31 is 1-4C-alkyl or 3-7C-cycloalkyl,
   R32 is hydrogen or 1-4C-alkyl,
   or where
   R31 and R32 together and including the nitrogen atom to which they are attached form a azetidino radical,
Arom is phenyl,
with the proviso that
when
R2 is 1-4C-alkyl
then
R3 is the radical —CO—NR31R32 or the radical CS—NR31R32,
   where for —CO—NR31R32
   R31 is 3-7C-cycloalkyl,
   R32 is hydrogen,
   and for —CS—NR31R32
   R31 is 1-4C-alkyl
   R32 is 1-4C-alkyl
   or where
   R31 and R32 together and including the nitrogen atom to which they are attached form a azetidino radical,
      and their salts.

Particular emphasis is also given to compounds of the formula 1a, where
R1 is 1-4C-alkyl,
R2 is 1-4C-alkyl
R3 is the radical —CO—NR31R32 or the radical CS—NR31R32,
   where for —CO—NR31R32
   R31 is 3-7C-cycloalkyl,
   R32 is hydrogen,
   and for —CS—NR31R32
   R31 is 1-4C-alkyl
   R32 is 1-4C-alkyl
   or where
   R31 and R32 together and including the nitrogen atom to which they are attached form a azetidino radical,
Arom is phenyl,
   and their salts.

Particular emphasis is also given to compounds of the formula 1a, where
R1 is 1-4C-alkyl,
R2 is 1-4C-alkylcarbonyl,
R3 is the radical —CO—NR31R32,
   where
   R31 is 1-4C-alkyl,
   R32 is 1-4C-alkyl,
Arom is phenyl,
   and their salts.

Particular emphasis is also given to compounds of the formula 1a, where
R1 is 1-4C-alkyl,
R2 is 1-4C-alkyl, hydroxy-3-4C-alkinyl, carboxyl, mono- or di-1-4C-alkylamino-1-4C-alkyl, 1-4C-alkylcarbonyl, 2-4C-alkinylcarbonyl or the radical —CO—NR21R22,
   where
   R21 is hydrogen, 1-4C-alkyl or 1-4C-alkoxy-1-4C-alkyl and
   R22 is hydrogen, 1-4C-alkyl or 1-4C-alkoxy-1-4C-alkyl,
R3 is a oxazolyl radical or the radical —CO—NR31R32,
   where
   R31 is 1-4C-alkyl or 3-7C-cycloalkyl
   R32 is hydrogen or 1-4C-alkyl,
   or where
   R31 and R32 together and including the nitrogen atom to which they are attached form a aziridino or azetidino radical,
Arom is phenyl,
with the proviso that
when
R2 is 1-4C-alkyl
then
R3 is a oxazolyl radical or the radical —CO—NR31R32,
   where
   R31 is 3-7C-cycloalkyl
   R32 is hydrogen

or where
R31 and R32 together and including the nitrogen atom to which they are attached form a aziridino or azetidino radical,
   and their salts.

Particular emphasis is also given to compounds of the formula 1a, where
R1 is 1-4C-alkyl,
R2 is hydroxy-3-4C-alkinyl, carboxyl, mono- or di-1-4C-alkylamino-1-4C-alkyl, 1-4C-alkylcarbonyl, 2-4C-alkinylcarbonyl or the radical —CO—NR21R22,
   where
   R21 is hydrogen, 1-4C-alkyl or 1-4C-alkoxy-1-4C-alkyl and
   R22 is hydrogen, 1-4C-alkyl or 1-4C-alkoxy-1-4C-alkyl,
R3 is the radical —CO—NR31R32,
   where
   R31 is 1-4C-alkyl,
   R32 is 1-4C-alkyl,
Arom is phenyl,
   and their salts.

Particular emphasis is also given to compounds of the formula 1a, where
R1 is 1-4C-alkyl,
R2 is 1-4C-alkyl,
R3 is a oxazolyl radical or the radical —CO—NR31R32,
   where
   R31 is 3-7C-cycloalkyl
   R32 is hydrogen,
   or where
   R31 and R32 together and including the nitrogen atom to which they are attached form a aziridino or azetidino radical,
Arom is phenyl,
   and their salts.

Particular emphasis is also given to compounds of the formula 1a, where
R1 is 1-4C-alkyl
R2 is carboxyl, mono- or di-1-4C-alkylamino-1-4C-alkyl or the radical —CO—NR21R22,
   where
   R21 is hydrogen, 1-4C-alkyl or 1-4C-alkoxy-1-4C-alkyl and
   R22 is hydrogen, 1-4C-alkyl or 1-4C-alkoxy-1-4C-alkyl,
R3 is the radical —CO—NR31R32,
   where
   R31 is 1-4C-alkyl and
   R32 is 1-4C-alkyl
Arom is phenyl
   and their salts.

The compounds of the formula 1 according to the invention can be synthesized from the corresponding starting compounds, for example according to the reaction scheme 1 given below. The synthesis is carried out in a manner known to the expert, for example as described in more detail in the examples which follow the schemes.

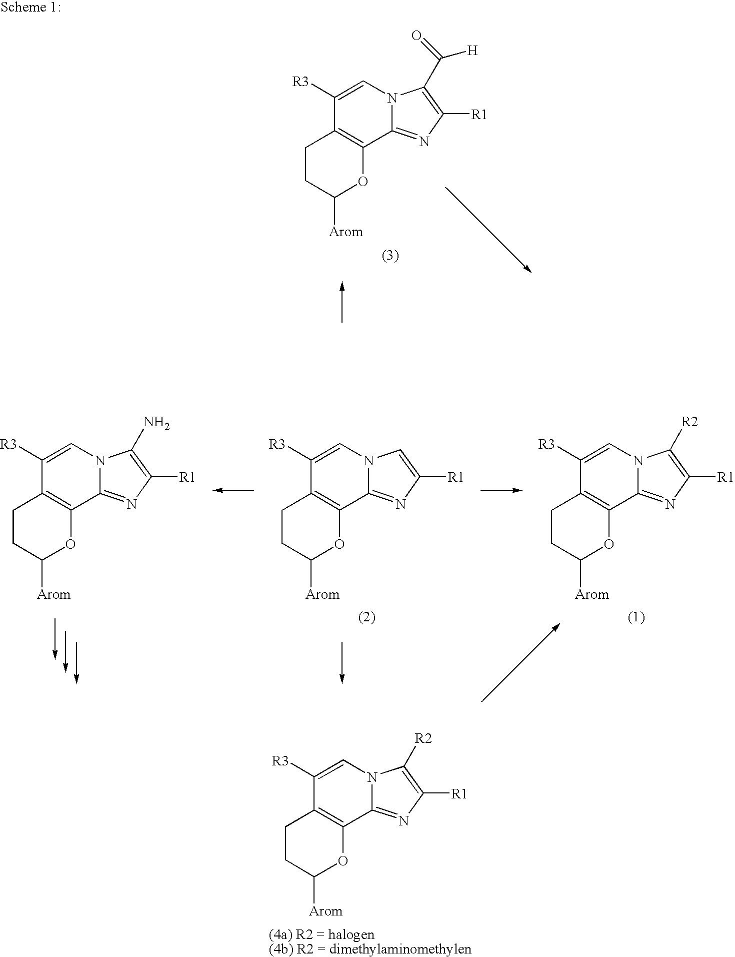

Compounds of the formula 2 can be transformed directly to compounds of the formula 1, for example by electrophilic aromatic substitution. Examples to be mentioned are aminoalkylation or halogenation reactions for the synthesis of compounds of the formula 1 with, for example, R2=mono- or di-1-4C-alkylaminomethyl or halogen.

Alternatively, compounds of the formula 2 can be first transformed, for example by a Vilsmeier formylation, to compounds of the formula 3, followed by further derivatization reactions, which are known to the expert (for example reduction of the carbonyl group, followed if desired by an etherification, or oxidation of the formyl functionality to a carboxylic acid, followed if desired by reaction with a suitable amine and formation of an amide group R2=—CO—NR21R22, or addition of Grignard reagents, followed if desired by an oxidation of the secondary hydroxy group), which lead to compounds of the formula 1.

Another possible access to compounds of the formula 1 is, for example, offered by the transformation of compounds of the formula 4a, for example by C—C-bond forming reactions, like for example Heck-, Suzuki- or Sonogashira-coupling reactions, followed, if desired, by further derivatization reactions known to the expert, like for example reduction of unsaturated substitutents R2 to the corresponding 1-4C-alkyl chains. Compounds of the formula 4a can be prepared from compounds of the formula 2 for example by a halogenation reaction, for example a bromination reaction using a bromination reagent, like for example N-bromosuccinimide.

Compounds of the formula 1 can also be obtained by treatment of compounds of the formula 4b with an alkylation agent, e.g. methyl iodide, and subsequent nucleophilic substitution of the quartary ammonium group, e.g. vs. cyanide. Compounds of the formula 4b can be prepared for example from compounds of the formula 2 by electrophilic substitution with Eschenmoser's salt.

Still another access to compounds of the formula 1 is, for example, offered by the transformation of compounds of the formula 2 to compounds of the formula 1 with R2=NH₂. This transformation can be achieved for example in analogy to the reactions described in J. Med. Chem., 1989, 32, 1686 or by nitration of compounds of the formula 2 and subsequent reduction of the nitro group. Further transformations by reactions known to the expert can then lead, if desired, to compounds of the formula 1 with R2=mono- or di-1-4C-alkylamino, 1-4C-alkylcarbonylamino, 1-4C-alkoxycarbonylamino or 1-4C-alkoxy-1-4C-alkoxycarbonylamino. Alternatively, compounds of the formula 1 with R2=NH₂ can be transformed into the corresponding diazonium salts. Further compounds of the formula 1, for example where R2 is e.g. hydroxy or 1-4C-alkoxy, can then be obtained by substitution of the diazonium group via reactions known to the expert.

Compounds of the formula 2 can be prepared, for example according to the reaction sequence outlined in scheme 2.

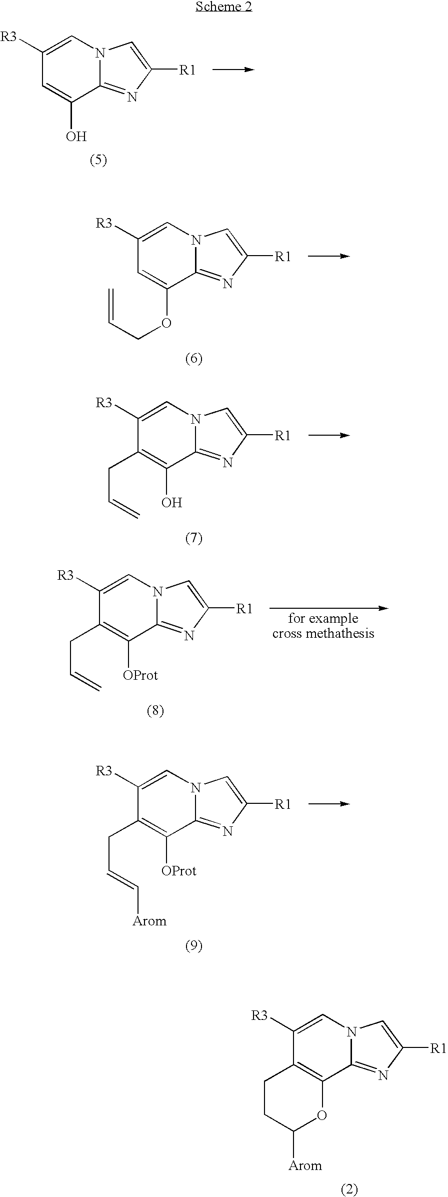

Compounds of the formula 7 can be obtained for example from compounds of the formula 5 by an O-alkylation followed by a thermally induced Claisen-rearrangement reaction of the O-alkylation product of the formula 6. Protection of the alcohol functionality in compounds of the formula 7 with a suitable protection group Prot, for example a pivaloyl group, using standard conditions leads to compounds of the formula 8, which can be subjected in a next reaction step for example to a cross metathesis reaction, for example using a suitable Grubbs catalyst, suitable for the introduction of the Arom residue. The reaction products of the formula 9 can be deprotected and the ring closure can be performed using methods known to the expert, for example under acidic conditions, which leads to the desired compounds of the formula 2.

Compounds of the formula 5 can be prepared as outlined in an exemplary manner in scheme 3.

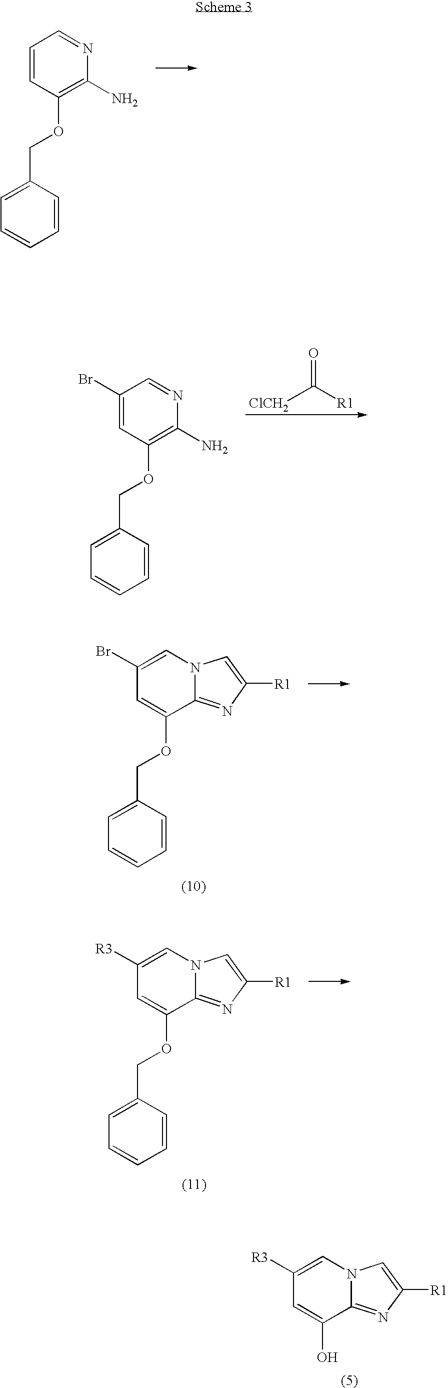

The preparation of compounds of the formula 11 from compounds of the formula 10 is carried out in a manner known per se to the person skilled in the art, for example in analogy to the reactions described in an exemplary manner in the International Patent Application WO 03/014123. Hydrogenation of compounds of the formula 11 to compounds of the formula 5 is carried out in a manner known per se to the person skilled in the art, using standard reaction conditions, like for example hydrogen/Pd(0).

Alternatively, compounds of the formula 1 can be prepared in a stereoselective way following the reaction steps as outlined generally in scheme 4. Compounds of the formula 13 can be prepared by asymmetric reduction of compounds of the formula 12. Numerous methods to perform asymmetric reduction of prochiral ketones are known (see for example E. N. Jacobsen, A. Pfaltz, Y. Yamamoto, Comprehensive Asymmetric Catalysis, Vol. I-III, Springer, Berlin, 1999) which comprise inter alia catalytic hydrogenation, catalytic transfer hydrogenation, chiral reducing agents (e.g. chiral boranes), achiral reducing agents in the presence of a chiral auxiliary or a chiral catalyst, hydrosilylation (achiral silane in combination with a chiral catalyst), and enzymatic reduction. The asymmetric catalytic hydrogenation using chiral hydrogenation catalysts of the Noyori type (RuCl₂[PP][NN]) is the preferred method for the synthesis of enantiopure diols of the formula 13. In the generic formula RuCl₂[PP][NN], PP is used as a general abbreviation for a chiral diphosphine ligand and NN is used as an abbreviation for a chiral diamine ligand. A detailed description of the method and specific examples of hydrogenation catalysts can be found for example in Angew. Chem. 2001, 113, 40-75 and in the literature cited therein. Transformation of derivatives of the formula 13 into enantiopure 7H-8,9-dihydro-pyrano[2,3-c]-imidazo[1,2-a]pyridines of the formula 1-a can be accomplished by methods which proceed under S_{N}2 conditions. For this purpose, the hydroxyl group in alpha-position to the Arom radical can be transformed into a suitable leaving group LG, e.g. by esterification with acid halides or sulfonyl chlorides. For the preparation of compounds of the formula 14a, the phenolic hydroxy group can be temporarily protected. Suitable protecting groups are described for example in T. W. Greene, P. G. M. Wuts “Protective Groups in Organic Synthesis” 3^{rd} edition, J. Wiley & Sons, New York, 1999. Alternatively, the phenolic hydroxyl group in compounds of the formula 13 can be transformed into a suitable leaving group LG using for example the reagents mentioned above leading to compounds of the formula 14b. A related procedure is disclosed in the International Patent Application WO 95/27714. Enantiopure compounds of the formula 1-a can be obtained, e.g. by heating of solutions of these intermediates 14a or 14b in dipolar aprotic solvents, like DMF or DMSO. The cyclization of compounds of the formula 14b can be carried out for example in the presence of a base, like e.g. sodium hydride. More conveniently, cyclization of the diols of the formula 13 can be accomplished under Mitsunobu conditions, e.g. using diisopropyl azodicarboxylate and triphenylphosphine.

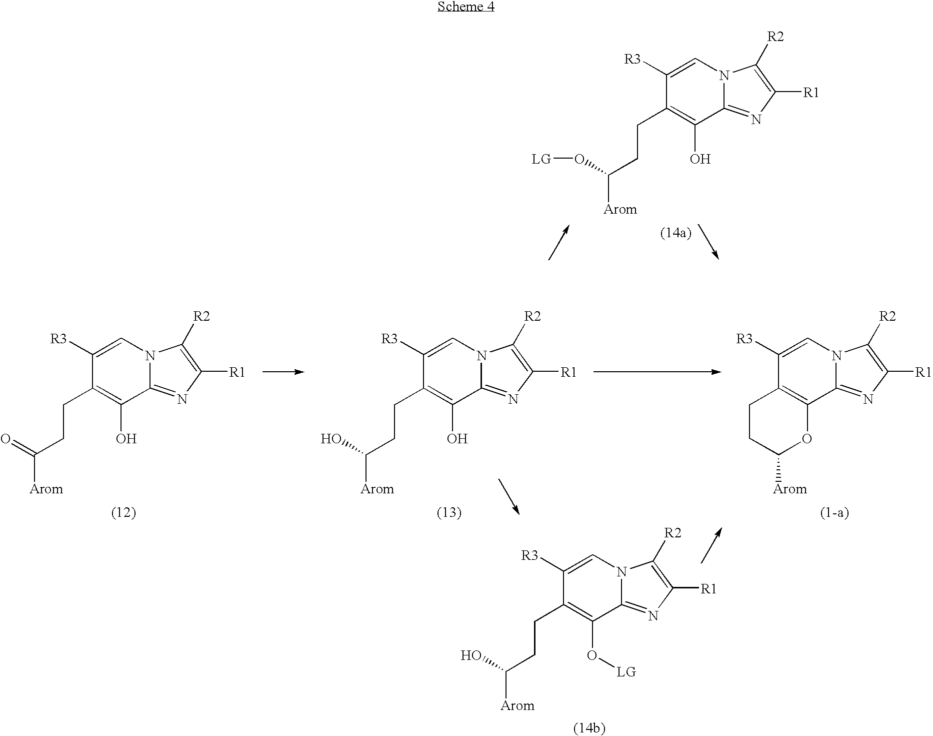

Compounds of the formula 12 are known for example from WO 03/014123, or they can be prepared in a known manner, analogously to known compounds. The purity of the compounds of the formula 12 has a major impact on the reaction conditions and the outcome of the asymmetric catalytic hydrogenation to compounds of the formula 13. In contrast to WO 03/014123 a further purification step is required, for example a crystallization step in the presence of a suitable organic acid. A convenient method to transform compounds of the formula 12 into other compounds of the formula 12 bearing a different substitutent R3 is shown in scheme 5 and might be illustrated by the following examples: Esters of 7-(3-aryl-3-oxo-propyl)-8-hydroxy-imidazo[1,2-a]pyridine-6-carboxylates of the formula 15, wherein R33 is for example a 1-4C-alkyl radical, can be transformed into acetals of the formula 16, for example by reaction with 2,2-dimethoxypropane in the presence of acids. Cleavage of the ester function, e.g. by saponification with sodium hydroxide, furnishes the corresponding carboxylic acids of the formula 17, which are then treated with a suitable coupling reagent, e.g. TBTU, followed by addition of the coupling partner, e.g. an amine, yielding derivatives of the formula 18. Alternatively, esters of the formula 16 can be reduced to the corresponding primary alcohol, e.g. using lithium aluminium hydride, and the hydroxyl group can be activated for example by conversion into a halide or a sulfonate using e.g. thionyl chloride or methanesulfonyl chloride. Interconversion of the substitutent R3 can then be accomplished by nucleophilic displacement reactions using nucleophiles like e.g. alkoxides. Finally, ketones of the formula 12 are obtained by cleavage of acetals of the formula 18, e.g. in the presence of acids like hydrochloric acid.

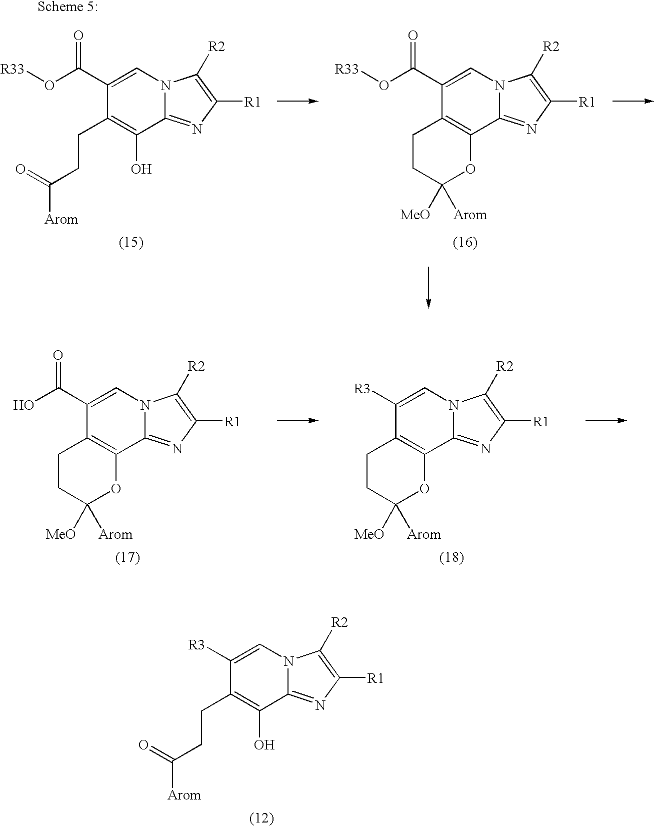

Another method suitable for asymmetric synthesis of compounds of the formula 1-a is depicted in Scheme 6. Compounds of the formula 19, which are obtained from compounds of the formula 9 by deprotection methods known to the person skilled in the art, can be transformed into chiral diols of the formula 13, for example by hydroboration of the double bond. Chiral reagents, which are suitable for this transformation, are discussed for example in Aldrichimica Acta 1987, 20(1), 9-24. An example that might be mentioned is isopinocampheylborane. Alternatively, achiral hydroboration reagents can be used in combination with a chiral catalyst. The transformation of chiral diols of the formula 13 into compounds of the formula 1-a was described above.

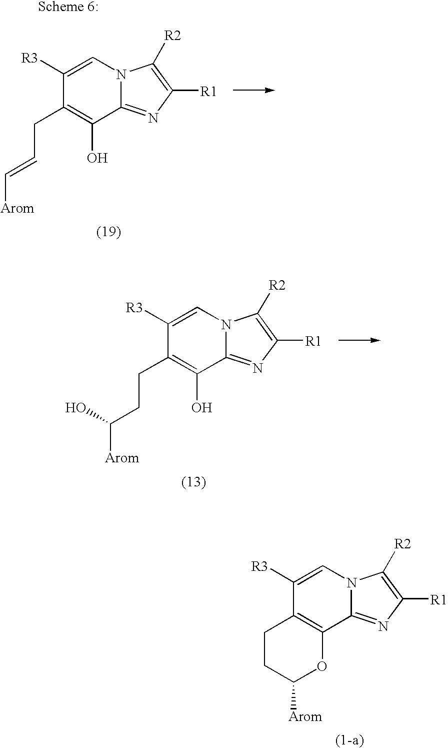

Likewise, the optical antipodes of the formula 1-b can be prepared in a stereoselective manner employing the methods, which are described above and illustrated in the schemes above. For this purpose, the transformations have to be conducted using the corresponding enantiomer of the chiral catalyst/chiral reagent, respectively.

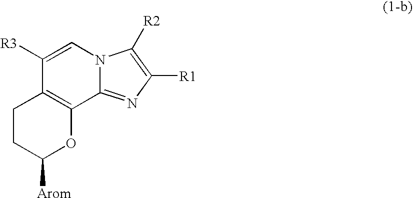

Another way to prepare compounds of the formula 1 is to reduce ketones of the formula 12, using e.g. sodium borohydride, followed by cyclization of the obtained diols, which might be accomplished by acid catalysis or under Mitsunobu conditions (see e.g. WO 03/014123).

The derivatization, if any, of the compounds of the formula 1 and of compounds obtained according to the above Schemes 1 to 6 (e.g. conversion of a group R3 into another group R3 or conversion of a group R2 into another group R2) is likewise carried out in a manner known to the expert. For example, if compounds where R2 and/or R3=—CO-1-4C-alkoxy, or where R3=—CO—NR31R32 are desired, an appropriate derivatization can be performed in a manner known to the expert (e.g. metal catalysed carbonylation of the corresponding halo compound or conversion of an ester into an amide), for example at the stage of an intermediate compound or more conveniently at a later point in time, for example conversion of a compound of the formula 1 into another compound of the formula 1.

Specific examples of such transformations are shown in scheme 7 and comprise e.g. condensation reactions between carboxylic acids of the formula 20 and N-nucleophiles, which might be mediated e.g. by TBTU (O-benzotriazol-1-yl-N,N,N′,N′-tetramethyluronium tetrafluoroborate) or CDI (N,N′-carbonyldiimidazole). Specific examples for N-nucleophiles are amines, sulfoneamines, hydroxylamines, and hydrazines. The compounds of the formula 21 are specific representatives of compounds of the formula 1 and/or are valuable intermediates for the preparation of such derivatives. Examples for further transformations of compounds of the formula 21 are the exchange of oxygen vs. sulfur or the N—OH group, e.g. using Lawesson's reagent or hydroxylamines, and elimination reactions, e.g. affording compounds where R3 is a nitrile group or a heterocyclic residue, e.g. a dihydrooxazole or a oxadiazole residue. Nitriles of the formula 22 can be converted into derivatives of the formula 1, where R3 is a heterocyclic group, e.g. a dihydrooxazole, dihydroimidazole, or tetrazole group. Compounds of the formula 23, where the R3 substitutent is a bromo atom, can also be considered as valuable intermediates for the synthesis of compounds of the formula 1 bearing different residues R3. A variety of different substitutents are accessible, e.g. by Palladium-catalyzed cross-coupling reactions using e.g. boronic acids, organotin derivatives, metal nitriles, alkenes, alkines, and combinations of carbon monoxide with amines/alcohols. If desired, the obtained compounds of the formula 1 can be transformed further by methods known to the person skilled in art. Specific examples of suitable transformations are described in the examples which follow without being limited to those.

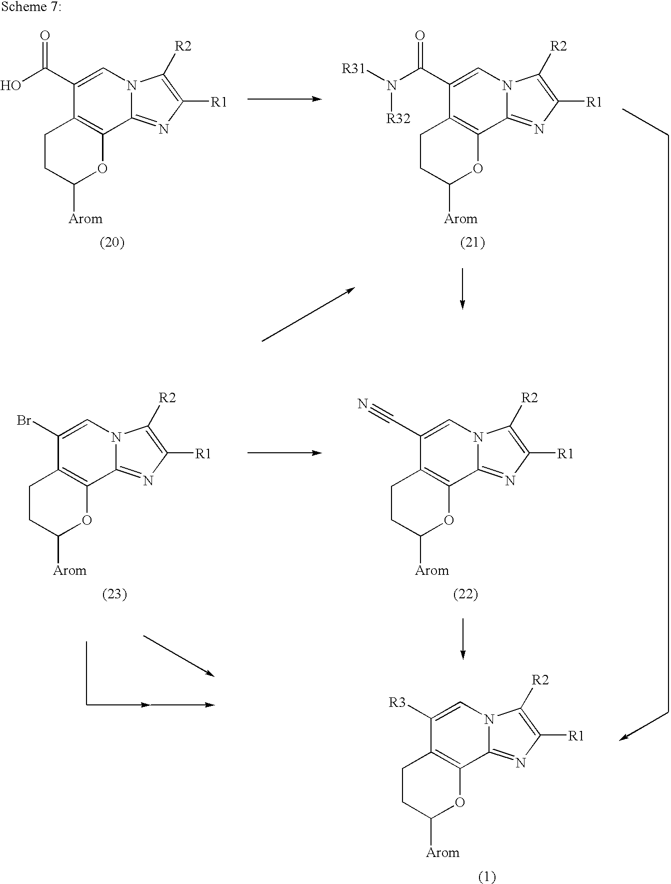

The invention further relates to a process for the synthesis of a compound of the formula 1, which comprises converting a compound of the formula 2, in which R1, R3 and Arom have the meanings as indicated in the outset, 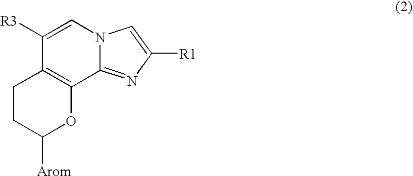
to a compound of the formula 1 wherein R1, R2, R3 and Arom have the meanings as indicated in the outset.

The invention further relates to a process for the synthesis of a compound of the formula 1-a which comprises,
an asymmetric reduction of a compound of the formula 12 to a compound of the formula 13 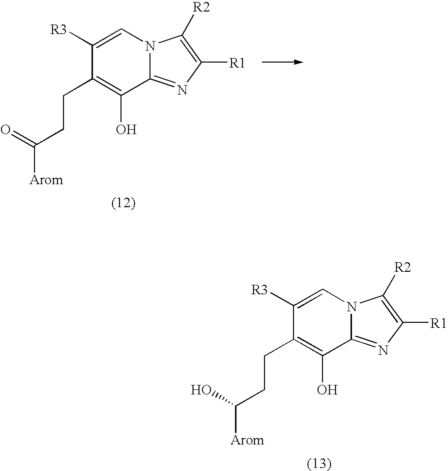
in which
R1, R2, R3 and Arom have the meanings as indicated in the outset
and conversion of a compound of the formula 13 into a compound of the formula 1-a or its salts.

The invention further relates to a process for the synthesis of a compound of the formula 1-a, which comprises
conversion of a compound of the formula 19 to a compound of the formula 13 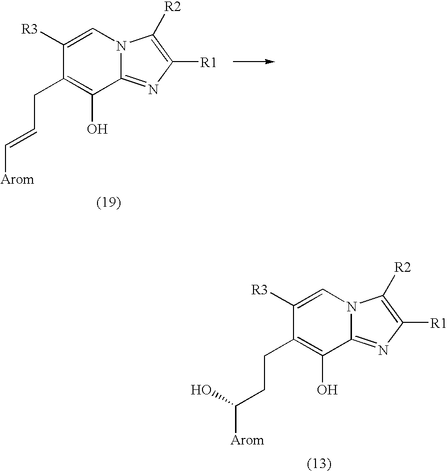
in which
R1, R2, R3 and Arom have the meanings as indicated in the outset
and conversion of a compound of the formula 13 into a compound of the formula 1-a or its salts.

The examples below serve to illustrate the invention in more detail without limiting it. Further compounds of the formula 1 whose preparation is not described explicitly can likewise be prepared in an analogous manner or in a manner known per se to the person skilled in the art, using customary process techniques. The abbreviation ee stands for enantiomeric excess, RT for retention time, SIC for substrate to catalyst ratio, TLC for thin layer chromatography, v for volume. For the assignment of NMR signals, the following abbreviations are used: s (singlet), d (duplet), t (triplet), q (quartet), m_{c} (multiplet centred), b (broad). The following units are used: ml (millilitre), l (litre), nm (nanometer), mm (millimeter), mg (milligramme), g (gramme), mmol (millimol), N (normal), M (molar), min (minute), MHz (megahertz).

Furthermore the following abbreviations are used for the chemical substances indicated:
DMSO dimethylsulfoxide
THF tetrahydrofuran
DMF dimethylformamide
DBU 1,8-diazabicyclo[5.4.0]undec-7-ene
TBTU O-benzotriazol-1-yl-N,N,N′,N′-tetramethyluronium tetrafluoroborate

The optical purity of the compounds of the formulae 1-a and 1-b was determined by capillary electrophoresis (CE) and/or high pressure liquid chromatography (HPLC). The experimental conditions for the separation of the enantiomers by HPLC are given for each example in the experimental section. The separation by CE was performed using one of the following experimental set-up:
Instrument: Agilent CE-3D
Capillary: 64.5 cm×75 μm, bubble-cell (Agilent)
Buffer: 50 mM sodium phosphate, pH 2.5 (Agilent)
Chiral selector: 40 mM heptakis(2,3,6-tri-O-methyl)-β-cyclodextrin (Cyclolab)
Voltage: 30 kV
Temperature: 10° C.

All of the HPLC columns used for preparative and analytical purposes are commercially available:
CHIRALPAK® AD, CHIRALPAK® AD-H, CHIRALPAK® 50801: DAICEL Chemical Industries Ltd, Tokyo or Chiral Technologies-Europe SARL, Ilkirch, France

If melting points were determined after crystallization of the compound, the solvent/solvent mixture that had been used for the purification is given in parentheses. If NMR (nuclear magnetic resonance) chemical shifts are given without integration, overlay of the signal of the corresponding proton of the compound with signals of the solvent, water, or impurities was observed.

I. Compounds of the Formula 1

### 1. 3-Dimethylaminomethyl-2-methyl-9-phenyl-7H-8,9-dihydro-pyrano[2,3-c]imidazo-[1,2-a]pyridine-6-carboxylic acid dimethylamide, Iodide Salt

2-Methyl-9-phenyl-7H-8,9-dihydro-pyrano[2,3-c]imidazo[1,2-a]pyridine-6-carboxylic acid dimethylamide (example ix, 0.250 g, 0.75 mmol) was dissolved in dry dichloromethane (10 ml) and N,N-dimethylmethyleneiminium iodide (0.138 g, 0.75 mmol) was added. The reaction mixture was stirred for 30 minutes at room temperature and was then evaporated to dryness. A colourless solid remained which was dried in vacuo. Thus, 0.377 g of the title compound was obtained (97% yield).

Melting point: 183-184° C.

¹H NMR (dmso-d₆, 200 MHz): δ=2.14, 2.27 (2 m_{c}, 2H), 2.40 (s, 3H), 2.55 (bs), 2.77, 2.90 (bs, s, 10H), 3.04 (s, 3H), 4.64 (bs, 2H), 5.31 (dd, 1H), 7.43 (m_{c}, 5H), 8.29 (s, 1H), 9.59 (bs, 1H).

### 2. 6-Dimethylcarbamoyl-2-methyl-9-phenyl-7H-8,9-dihydro-pyrano[2,3-c]imidazo[1,2-a]pyridine-3-carboxylic acid

A solution of 3-formyl-2-methyl-9-phenyl-7H-8,9-dihydro-pyrano[2,3-c]imidazo[1,2-a]pyridine-6-carboxylic acid dimethylamide (example xi, 1.10 g, 3.0 mmol) in THF (30 ml) and water (20 ml) was treated with sulfamic acid (0.50 g, 5.1 mmol) and was cooled to 0° C. An aqueous solution (5 ml) of sodium chlorite (80% purity, 0.47 g, 4.2 mmol) was added dropwise. The reaction mixture was stirred for 1.25 hours at 0° C. After addition of an aqueous solution (5 ml) of sodium sulfite (0.65 g, 5.2 mmol) stirring was continued for 5 minutes. The reaction mixture was extracted with dichloromethane (2×50 ml). The organic phases were dried over sodium sulfate and concentrated under reduced pressure. The residue (750 mg) was dissolved in dichloromethane (10 ml) and water (10 ml). A pH-value of 8 was adjusted by addition of 2 N sodium hydroxide solution (0.6 ml). The phases were separated and the aqueous phase was extracted with dichloromethane (2×10 ml). The organic phases were discarded and the aqueous phase was acidified to pH 5 by addition of 2 N hydrochloric acid (1 ml). The aqueous phase was extracted with dichloromethane (2×20 ml), diluted with saturated sodium chloride solution (5 ml), and extracted again with another portion of dichloromethane. The combined dichloromethane phases were dried over sodium sulfate and concentrated under reduced pressure to yield the title compound (450 mg of a colourless solid, 39% yield). The aqueous phase was concentrated to a volume of 5 ml. After addition of dichloromethane (10 ml) the pH-value was re-adjusted to 5 by addition of 2 N hydrochloric acid (0.5 ml). Following the procedure described above, another 300 mg of the title compound were obtained (26% yield).

Melting point: 138° C.

¹H NMR (CDCl₃, 200 MHz): δ=2.31 (m_{c}, 2H), 2.69, 2.74 (m_{c}, s, 4H), 2.91, 2.96 (m_{c}, s, 4H), 3.16 (s, 3H), 5.33 (dd, 1H), 7.29 (m_{c}), 7.43 (m_{c}, 2H), 8.93 (s, 1H).

### 3. 2-Methyl-9-phenyl-7H-8,9-dihydro-pyrano[2,3-c]imidazo[1,2-a]pyridine-3,6-dicarboxylic acid bis-dimethylamide

A solution of 6-dimethylcarbamoyl-2-methyl-9-phenyl-7H-8,9-dihydro-pyrano[2,3-c]imidazo[1,2-a]pyridine-3-carboxylic acid (example 2, 0.120 g, 0.32 mmol) in dichloromethane (20 ml) was treated with TBTU (0.107 g, 0.33 mmol). The suspension was stirred for 1 hour at room temperature. A 2 M solution of dimethylamine in THF (0.32 ml, 0.64 mmol) was added and stirring was continued for 1.5 hours at room temperature. The reaction mixture was quenched by addition of water (20 ml). The phases were separated and the aqueous phase was extracted with dichloromethane (2×10 ml). The combined organic phases were dried over sodium sulfate and concentrated under reduced pressure. A yellowish solid (0.124 g) remained which was dried in vacuo. The title compound was isolated in 97% yield.

Melting point: 190° C.

¹H NMR (CDCl₃, 200 MHz): δ=2.26 (m_{c}, 2H), 2.47 (s, 3H), 2.61 (m_{c}, 1H), 2.80 (m_{c}), 2.95 (s, 3H), 3.10, 3.12 (2s, 9H), 5.33 (dd, 1H), 7.39 (m_{c}, 5H), 8.06 (s, 1H).

### 4. 2-Methyl-9-phenyl-7H-8,9-dihydro-pyrano[2,3-c]imidazo[1,2-a]pyridine-3,6-dicarboxylic acid 3-[(2-methoxyethyl)amide]6-dimethylamide

6-Dimethylcarbamoyl-2-methyl-9-phenyl-7H-8,9-dihydro-pyrano[2,3-c]imidazo[1,2-a]pyridine-3-carboxylic acid (example 2, 0.200 g, 0.53 mmol) was dissolved in dichloromethane (30 ml) and was treated with TBTU (0.177 g, 0.55 mmol). The suspension was stirred for 1 hour at room temperature. Methoxyethylamine (0.130 g, 1.73 mmol) was added and the reaction was continued for 1 hour at room temperature. The reaction was quenched by addition of water (20 ml). The phases were separated and the aqueous phase was extracted with dichloromethane (2×20 ml). The combined organic phases were dried over sodium sulfate and concentrated under reduced pressure. The crude product (0.21 g) was purified by flash chromatography [6 g of silica gel, eluant: ethyl acetate/methanol=95:5 (v/v)]. A colourless solid (0.16 g, 70% yield) was isolated, which was the pure title compound.

Melting point: 208° C.

¹H NMR (CDCl₃, 200 MHz): δ=2.27 (m_{c}, 2H), 2.61, 2.71 (m_{c}, s, 4H), 2.84, 2.96 (m_{c}, s, 4H), 3.11 (s, 3H), 3.42 (s, 3H), 3.64 (m_{c}, 4H), 5.32 (dd, 1H), 6.23 (bt, 1H), 7.39 (m_{c}, 5H), 9.01 (s, 1H).

### 5. 3-(1-Hydroxy-2-butynyl)-2-methyl-9-phenyl-7H-8,9-dihydro-pyrano[2,3-q]imidazo[1,2-a]pyridine-6-carboxylic acid dimethylamide

In a flame-dried flask filled with argon, 3-formyl-2-methyl-9-phenyl-7H-8,9-dihydro-pyrano[2,3-c]imidazo[1,2-a]pyridine-6-carboxylic acid dimethylamide (example xi, 1.00 g, 2.8 mmol) was suspended in dry THF (50 ml). The suspension was cooled to −78° C. and propinylmagnesium bromide (11.0 ml of a 0.5 M solution in THF, 5.5 mmol) was added using a syringe. The reaction mixture was stirred for 1 hour at −78° C. and for 2 hours at 0° C. and was then quenched by addition of water (30 ml) and dichloromethane (70 ml). The phases were separated and the aqueous phase was extracted with dichloromethane (2×50 ml). The combined organic phases were washed with water (20 ml) and saturated sodium chloride solution (20 ml), dried over sodium sulfate, and concentrated in vacuo. A yellow foamy solid (1.07 g, 96% yield) was isolated which was characterized by ¹H-NMR spectroscopy as an almost pure diasteromeric mixture of the title compound.

¹H-NMR (CDCl₃, 200 MHz): δ=1.84, 1.85 (2 s), 2.25 (m_{c}, 2H), 2.39 (s, 3H), 2.60, 2.81 (2 m_{c}, 2H), 2.93, 2.96 (2 s, Σ 3H), 3.12 (s, 3H), 3.74 (m_{c}), 5.30 (m_{c}, 1H), 5.85 (m_{c}, 1H), 7.38 (m_{c}, 5H), 8.14, 8.15 (2 s, Σ 1H).

### 6. 2-Methyl-3-(1-oxo-2-butynyl)-9-phenyl-7H-8,9-dihydro-pyrano[2,3-c]imidazo[1,2-a]pyridine-6-carboxylic acid dimethylamide

A solution of 3-(1-hydroxy-2-butynyl)-2-methyl-9-phenyl-7H-8,9-dihydro-pyrano[2,3-c]imidazo[1,2-a]pyridine-6-carboxylic acid dimethylamide (example 5, 500 mg, 1.24 mmol) in dichloromethane (20 ml) was treated with manganese dioxide (4.0 g, 46 mmol). The suspension was stirred for 1 hour at room temperature and was then filtered over Celite®. Concentration of the filtrate yielded a yellow foamy solid, which was purified by flash chromatography (silica gel, eluant: ethyl acetate). After evaporation of the corresponding fractions the title compound was isolated in 86% yield (430 mg of a yellow solid, almost pure by means of ¹H-NMR spectroscopy).

Melting point: 216-217° C.

¹H-NMR (CDCl₃, 200 MHz): δ=2.16 (s, 3H), 2.30 (m_{c}, 2H), 2.70 (m_{c}, 1H), 2.90, 2.91, 2.94 (s, m_{c}, s, 7H), 3.14 (s, 3H), 3.48 (s), 5.34 (dd, 1H), 7.39 (m, 5H), 9.28 (s, 1H).

### 7. 3-(1-Hydroxypropynyl)-2-methyl-9-phenyl-7H-8,9-dihydro-pyrano[2,3-c]imidazo[1,2-a]pyridine-6-carboxylic acid dimethylamide

In a flame-dried flask filled with argon, 3-formyl-2-methyl-9-phenyl-7H-8,9-dihydro-pyrano[2,3-c]imidazo[1,2-a]pyridine-6-carboxylic acid dimethylamide (example xi, 0.67 g, 1.8 mmol) was suspended in dry THF (50 ml). The suspension was cooled to −78° C. and ethinylmagnesium bromide (7.4 ml of a 0.5 M solution in THF, 3.7 mmol) was added using a syringe. The reaction mixture was stirred for 1 hour at −78° C. and for 2 hours at 0° C. and was then quenched by addition of water (40 ml) and dichloromethane (60 ml). The phases were separated and the aqueous phase was extracted with dichloromethane (2×40 ml). The combined organic phases were washed with saturated sodium chloride solution (20 ml), dried over sodium sulfate, and concentrated in vacuo. The crude product was purified by flash chromatography [15 g of silica gel, eluant: dichloromethane/methanol=100:1 (v/v)]. A colourless foamy solid (0.63 g, 88% yield) was isolated which was characterized by ¹H-NMR spectroscopy as a pure diasteromeric mixture of the title compound.

¹H-NMR (CDCl₃, 200 MHz): δ=2.33 (m_{c}, s, 5H), 2.56, 2.58 (2 m_{c}, 2H), 2.79 (m_{c}, 1H), 2.93 (s, 3H), 3.11 (s, 3H), 5.30 (m_{c}, 1H), 5.86 (m_{c}, 1H), 7.38 (m_{c}, 5H), 8.11 (2 s, Σ 1H).

### 8. 2-Methyl-3-(1-oxopropynyl)-9-phenyl-7H-8,9-dihydro-pyrano[2,3-c]imidazo[1,2-a]pyridine-6-carboxylic acid dimethylamide

A solution of 3-(1-hydroxypropynyl)-2-methyl-9-phenyl-7H-8,9-dihydro-pyrano[2,3-c]imidazo[1,2-a]pyridine-6-carboxylic acid dimethylamide (example 7, 300 mg, 0.77 mmol) in dichloromethane (20 ml) was treated with manganese dioxide (2.4 g, 28 mmol). The suspension was stirred for 1 hour at room temperature and was then filtered over Celite®. Concentration of the filtrate yielded a yellow foamy solid, which was purified by flash chromatography (silica gel, eluant: ethyl acetate). After evaporation of the corresponding fractions the title compound was isolated in 67% yield (200 mg of a yellow solid).

Melting point: 220-222° C.

¹H-NMR (CDCl₃, 200 MHz): δ=2.29 (m_{c}, 2H), 2.71 (m_{c}, 1H), 2.92, 2.93, 2.94 (m, 2 s, 7H), 3.15 (s, 3H), 3.48 (s, 1H), 5.36 (dd, 1H), 7.39 (m_{c}, 5H), 9.26 (s, 1H).

### 9. 3-Acetyl-2-methyl-9-phenyl-7H-8,9-dihydro-pyrano[2,3-c]imidazo-[1,2-a]pyridine-6-carboxylic acid dimethylamide

2-Methyl-9-phenyl-7H-8,9-dihydro-pyrano[2,3-c]imidazo-[1,2-a]pyridine-6-carboxylic acid dimethylamide (example 1x, 1.10 g, 3.3 mmol) was dissolved in acetic anhydride (50 ml). After addition of methanesulfonic acid (0.38 g, 3.9 mmol), the solution was heated for 1.5 days at 140° C. The reaction mixture was concentrated and saturated sodium bicarbonate solution (90 ml) was added in order to adjust a pH-value of 7-8. The aqueous phase was extracted with dichloromethane (2×70 ml, 1×30 ml). The combined organic phases were dried over sodium sulfate and concentrated under reduced pressure. The brown residue was purified by flash chromatography (silica gel, eluant: ethyl acetate) yielding 0.57 g of the title compound (colourless solid, 46% yield).

Melting point: 249-251° C.

¹H-NMR (CDCl₃, 200 MHz): δ=2.29 (m_{c}, 2H), 2.61-3.00, 2.61, 2.80, 2.94 (m, 3 s, 11H), 3.14 (s, 3H), 5.34 (dd, 1H), 7.38 (m_{c}, 5H), 9.32 (s, 1H).

### 10. (2,3-Dimethyl-9-phenyl-7H-8,9-dihydro-pyrano[2,3-c]-imidazo[1,2-a]pyridin-6-yl)-aziridin-1-yl methanone

A suspension of 2,3-dimethyl-9-phenyl-7H-8,9-dihydro-pyrano[2,3-c]-imidazo[1,2-a]pyridine-6-carboxylic acid (example xiii, 0.50 g, 1.5 mmol) in dichloromethane (25 ml) was treated with TBTU (0.50 g, 1.6 mmol). After a reaction time of 50 minutes at reflux, the yellow suspension was cooled to room temperature and aziridine (60 mg, 1.39 mmol) was added. The reaction mixture was stirred for 40 minutes at room temperature, at which point a clear solution was obtained. The reaction mixture was poured onto saturated sodium bicarbonate solution, the phases were separated, and the aqueous phase was extracted with dichloromethane (2×20 ml). The combined organic phases were dried over sodium sulfate and concentrated in vacuo. The residue (0.75 g) was purified by flash chromatography [30 g of silica gel, eluant: dichloromethane/methanol=100:3 (v/v)]. A yellow oil was isolated which was treated with a mixture of acetone (5 ml), diethyl ether (5 ml) and methanol (1 drop). The pure title compound was obtained in 15% yield (82 mg of a colourless solid).

Melting point: 180-181° C. (acetone/diethyl ether)

¹H-NMR (CDCl₃, 200 MHz): δ=2.23 (m_{c}, 12H), 3.08 (m_{c}, 2H), 5.29 (dd, 1H), 7.39 (m_{c}, 5H), 8.31 (s, 1H).

### 11. (2,3-Dimethyl-9-phenyl-7H-8,9-dihydro-pyrano[2,3-c]-imidazo[1,2-a]pyridin-6-yl)-azetidin-1-yl methanone

A suspension of 2,3-dimethyl-9-phenyl-7H-8,9-dihydro-pyrano[2,3-c]-imidazo[1,2-a]pyridine-6-carboxylic acid (example xiii, 1.70 g, 5.3 mmol) in dichloromethane (50 ml) was treated with TBTU (1.85 g, 5.8 mmol). After a reaction time of 1.5 hours at reflux, azetidine (316 mg, 373 μl, 5.53 mmol) was added. The resulting solution was stirred for 2 hours at room temperature. The reaction was quenched with water (50 ml), the phases were separated, and the aqueous phase was extracted with dichloromethane (2×20 ml). The combined organic phases were dried over sodium sulfate and concentrated in vacuo. The residue (3.46 g of a foamy solid) was purified by flash chromatography [100 g of silica gel, eluant: dichloromethane/methanol=100:3 (v/v)]. The corresponding fractions were evaporated and the obtained solid was dissolved in a mixture of dichloromethane (50 ml) and water (25 ml). Sodium hydroxide solution (2 N) was added until a pH-value of 10 was obtained. The phases were separated and the aqueous phase was extracted with dichloromethane (20 ml). The combined organic phases were dried over sodium sulfate and concentrated under reduced pressure. This afforded the pure title compound [1.68 g of a colourless solid, 88% yield].

Melting point: 254° C.

¹H-NMR (CDCl₃, 200 MHz): δ=2.29, 2.37, 2.41 (m_{c}, 2 s, 10H), 2.76 (m, 1H), 2.99 (m_{c}, 1H), 4.18 (bs, 4H), 5.30 (dd, 1H), 7.38 (m_{c}, 6H).

### 12. (3-Hydroxy-azetidin-1-yl)-(2,3-dimethyl-9-phenyl-7H-8,9-dihydro-pyrano[2,3-c]-imidazo[1,2-a]pyridin-6-yl)-methanone

A suspension of 2,3-dimethyl-9-phenyl-7H-8,9-dihydro-pyrano[2,3-c]-imidazo[1,2-a]pyridine-6-carboxylic acid (example xiii, 0.400 g, 1.24 mmol) in dichloromethane (20 ml) was treated with TBTU (0.470 g, 1.46 mmol). After a reaction time of 2 hours at reflux, 3-hydroxyazetidine (95 mg, 1.30 mmol) and DMF (4 ml) was added at room temperature. The reaction mixture was heated for 2 hours at 50° C. The suspension was cooled to 0° C. and was poured onto a stirred mixture of sodium bicarbonate solution (20 ml) and dichloromethane (25 ml). Stirring was continued for several minutes, the phases were separated, and the aqueous phase was extracted with dichloromethane (3×10 ml). The combined organic phases were washed with water (2×10 ml), dried over sodium sulfate and concentrated in vacuo. The residue (1 g of a brown oil) was purified by flash chromatography [20 g of silica gel, eluant: dichloromethane/methanol=100:3 (v/v)]. The corresponding fractions were evaporated and the obtained solid was washed with diethyl ether (3 ml). This afforded the pure title compound [170 mg of a colourless solid, 36% yield].

Melting point: 306-308° C. (diethyl ether)

¹H-NMR (DMSO-d₆, 200 MHz): δ=2.10 (mc, 1H), 2.26 (s, mc, 4H), 2.37 (s, 3H), 2.66 (mc, 1H), 2.92 (mc, 1H), 3.85 (mc, 2H), 4.23 (mc, 2H), 4.50 (mc, 1H), 5.26 (dd, 1H), 5.75 (mc, 1H), 7.42 (mc, 5H), 7.85 (s, 1H).

### 13. 2,3-Dimethyl-9-phenyl-7H-8,9-dihydro-pyrano[2,3-c]-imidazo[1,2-a]pyridine-6-carboxylic acid cyclopropylamide

A suspension of 2,3-dimethyl-9-phenyl-7H-8,9-dihydro-pyrano[2,3-c]-imidazo[1,2-a]pyridine-6-carboxylic acid (example xiii, 1.70 g, 5.3 mmol) in dichloromethane (50 ml) was treated with TBTU (1.85 g, 5.8 mmol). After a reaction time of 1.5 hours at reflux, cyclopropylamine (314 mg, 381 μl, 5.50 mmol) was added. The resulting solution was stirred for 2 hours at room temperature. The reaction was quenched with water (50 ml), the phases were separated, and the aqueous phase was extracted with dichloromethane (2×20 ml). The combined organic phases were dried over sodium sulfate and concentrated in vacuo. The residue (3.2 g of a yellow foamy solid) was purified by flash chromatography [100 g of silica gel, eluant: dichloromethane/methanol=100:3 (v/v)]. The corresponding fractions were evaporated and the obtained sticky solid was suspended in a mixture of ethyl acetate (5 ml) and diethyl ether (40 ml). Stirring was continued for 1 hour at room temperature. The title compound was isolated by filtration and was dissolved in a mixture of dichloromethane (50 ml) and water (25 ml). Sodium hydroxide solution (2 N) was added until a pH-value of 10 was obtained. The phases were separated and the aqueous phase was extracted with dichloromethane (20 ml). The combined organic phases were dried over sodium sulfate and concentrated under reduced pressure. This afforded the pure title compound [0.86 g of a colourless solid, 45% yield, ¹H-NMR spectrum indicated the presence of methanol (7-8 weight-%)].

Melting point: 260° C.

¹H-NMR (dmso-d₆, 200 MHz): δ=0.57 (m_{c}, 2H), 0.70 (m_{c}, 2H), 2.06 (m_{c}, 1H), 2.26 (s, m_{c}, 4H), 2.37 (s, 3H), 2.66-3.08 (m, 3H), 3.17 (d, MeOH), 4.07 (q, MeOH), 5.23 (dd, 1H), 7.42 (m, 5H), 7.86 (s, 1H), 8.42 (d, 1H).

### 14. 2,3-Dimethyl-9-phenyl-7H-8,9-dihydro-pyrano[2,3-c]-imidazo[1,2-a]pyridine-6-carboxylic acid cyclobutylamide

A suspension of 2,3-dimethyl-9-phenyl-7H-8,9-dihydro-pyrano[2,3-c]-imidazo[1,2-a]pyridine-6-carboxylic acid (example xiii, 0.50 g, 1.5 mmol) in dichloromethane (25 ml) was treated with TBTU (0.50 g, 1.6 mmol). After a reaction time of 1 hour at reflux, the suspension was cooled to room temperature and cyclobutylamine (110 mg, 132 μl, 1.54 mmol) was added. The reaction mixture was stirred for 1 hour at room temperature and was then poured onto saturated sodium bicarbonate solution (50 ml). The phases were separated, and the aqueous phase was extracted with dichloromethane (2×10 ml). The combined organic phases were dried over sodium sulfate and concentrated in vacuo. The residue (0.62 g) was purified by flash chromatography [40 g of silica gel, eluant: dichloromethane/methanol=20:1 (v/v)]. The corresponding fractions were evaporated and the obtained solid (300 mg) was suspended in a mixture of acetone (20 ml) and diethyl ether (20 ml). Stirring was continued for 30 minutes at 0° C. and the pure title compound (270 mg, 47% yield) was isolated by filtration.

Melting point: 257-258° C. (acetone/diethyl ether)

¹H-NMR (CDCl₃, 200 MHz): δ=1.79 (m_{c}), 1.90-2.50, 2.33, 2.40 (m, 2 s, 12H), 2.84 (m_{c}, 1H), 3.01 (m, 1H), 4.55 (m_{c}, 1H), 5.22 (dd, 1H), 6.50 (d, 1H), 7.39 (m_{c}, 6H).

### 15. 2,3-Dimethyl-9-phenyl-7H-8,9-dihydro-pyrano[2,3-c]-imidazo[1,2-a]pyridine carboxylic acid phenylamide

A suspension of 2,3-dimethyl-9-phenyl-7H-8,9-dihydro-pyrano[2,3-c]-imidazo[1,2-a]pyridine-6-carboxylic acid (example xiii, 0.400 g, 1.24 mmol) in dichloromethane (20 ml) was treated with TBTU (0.470 g, 1.46 mmol). After a reaction time of 2 hours at reflux, aniline (120 l, 123 mg, 1.32 mmol) was added at room temperature. The suspension was stirred for 1 hour at room temperature and was diluted with DMF (6 ml). The reaction was continued for 1 hour at room temperature and for 30 minutes at 40° C. The brown suspension was cooled to 0° C. and was poured onto a stirred mixture of sodium bicarbonate solution (20 ml) and dichloromethane (20 ml). Stirring was continued for several minutes, the phases were separated, and the aqueous phase was extracted with dichloromethane (3×8 ml). The combined organic phases were washed with water (2×10 ml), dried over sodium sulfate, and concentrated in vacuo. The residue (900 mg of a yellow oil) was purified by flash chromatography [15 g of silica gel, eluant: dichloromethane/methanol=100:3 (v/v)]. The corresponding fractions were evaporated and the residue was dried in vacuo (390 mg of a yellow oil, 79% yield). The title compound was crystallized from acetone (1 ml), isolated by filtration, washed with cold acetone (1 ml) and diethyl ether (5 ml), and dried in vacuo. This afforded 190 mg of colourless crystals (39% yield).

Melting point: 285-287° C. (diethyl ether)

¹H-NMR (DMSO-d₆, 200 MHz): δ=2.09 (mc, 1H), 2.29 (mc, s, 4H), 2.41 (s, 3H), 2.77 (mc, 1H), 3.06 (mc, 1H), 5.28 (dd, 1H), 7.11 (t, 1H), 7.42 (mc, 7H), 7.73 (d, 2H), 8.14 (s, 5H), 10.37 (s, 1H).

### 16. 2,3-Dimethyl-9-phenyl-7H-8,9-dihydro-pyrano[2,3-c]-imidazo[1,2-a]pyridine-6-carboxylic acid (4-ethoxy-phenyl)-amide

A suspension of 2,3-dimethyl-9-phenyl-7H-8,9-dihydro-pyrano[2,3-c]-imidazo[1,2-a]pyridine-6-carboxylic acid (example xiii, 1.00 g, 3.1 mmol) in dichloromethane (50 ml) was treated with TBTU (1.08 g, 3.4 mmol). After a reaction time of 1 hour at room temperature, p-phenetidine (286 mg, 271 μl, 2.08 mmol) was added. The resulting solution was stirred for 2 hours at room temperature. More p phenetidine (143 mg, 135 μl, 1.04 mmol) was added and stirring was continued for 1 hour at room temperature. The reaction was quenched with sodium bicarbonate solution, the phases were separated, and the aqueous phase was extracted with dichloromethane (2×). The combined organic phases were dried over sodium sulfate and concentrated in vacuo. The residue (1.85 g) was crystallized from acetone/ethyl acetate/diethyl ether [2:10:10 (v/v/v)]. The resulting suspension was stirred for 1 hour at room temperature and the precipitate was isolated by filtration (880 mg). Upon concentration of the mother liquor more precipitate was formed, which was also isolated by filtration (193 mg). The two batches were combined and were purified by flash chromatography [silica gel, eluant: dichloromethane/methanol=100:3 (v/v)]. The corresponding fractions were evaporated and the obtained foamy solid was washed with diethyl ether. This afforded the pure title compound (670 mg, 49% yield).

Melting point: 223° C. (diethyl ether)

¹H-NMR (CHCl₃, 200 MHz): δ=1.42 (t, 3H), 2.11 (m_{c}, 1H), 2.27, 2.33, 2.39 (s, m_{c}, s, 7H), 2.89 (m, 1H), 3.10 (m_{c}, 1H), 4.04 (q, 2H), 5.14 (dd, 1H), 6.87 (d, 2H), 7.25 (m_{c}), 7.38 (m_{c}, 2H), 7.44 (s, 1H), 7.64 (d, 2H), 8.71 (bs, 1H).

### 17. N-(2,3-Dimethyl-9-phenyl-7H-8,9-dihydro-pyrano[2,3-c]-imidazo[1,2-a]pyridine-6-carbonyl)methanesulfonamide

A suspension of 2,3-dimethyl-9-phenyl-7H-8,9-dihydro-pyrano[2,3-c]-imidazo[1,2-a]pyridine-6-carboxylic acid (example xiii, 0.80 g, 2.5 mmol) in dry THF (30 ml) was treated with N,N′-carbonyldiimidazole (0.80 g, 4.9 mmol). After a reaction time of 2 hours at 40° C. a brown solution was obtained. DBU (0.75 g, 4.9 mmol) and methanesulfonamide (0.47 g, 4.9 mmol) was added and stirring was continued for 1 hour at room temperature. The reaction mixture was poured onto water (30 ml) and dichloromethane (50 ml), the phases were separated, and the aqueous phase was extracted with dichloromethane (30 ml). The combined organic phases were dried over sodium sulfate and concentrated in vacuo. The residue (1.5 g of a brown foamy solid) was purified by flash chromatography [45 g of silica gel, eluant: dichloromethane/methanol=100:3 (v/v)]. The corresponding fractions were evaporated and the title compound was isolated (0.70 g, 71% yield).

Melting point: 210° C.

¹H-NMR (dmso-d₆, 200 MHz): δ=2.09 (m_{c}, 1H), 2.30 (s, m_{c}, 4H), 2.41 (s, 3H), 3.00, 3.10 (s, m_{c}, 5H), 5.28 (dd, 1H), 7.44 (m_{c}, 5H), 8.14 (s, 1H).

### 18. (2,3-Dimethyl-9-phenyl-7H-8,9-dihydro-pyrano[2,3-c]-imidazo[1,2-a]pyridin-6-yl)-piperazin-1-yl-methanone

A suspension of 2,3-dimethyl-9-phenyl-7H-8,9-dihydro-pyrano[2,3-c]-imidazo[1,2-a]pyridine-6-carboxylic acid (example xiii, 0.90 g, 2.8 mmol) in dry THF (30 ml) was treated with N,N′-carbonyldiimidazole (0.91 g, 5.6 mmol). After a reaction time of 2 hours at 40° C. a brown solution was obtained. DBU (0.85 g, 5.6 mmol) and piperazine (0.48 g, 5.6 mmol) was added and stirring was continued for 2.5 days at room temperature. The reaction mixture was poured onto water (30 ml) and dichloromethane (50 ml), the phases were separated, and the aqueous phase was extracted with dichloromethane (30 ml). The combined organic phases were washed with water (20 ml), dried over sodium sulphate, and concentrated in vacuo. The residue (1.3 g) was purified by flash chromatography [40 g of silica gel, eluant: dichloromethane/methanol=100:5 (v/v)]. Evaporation of the corresponding fractions furnished a yellow sticky solid, which was washed with diethyl ether (30 ml). The precipitate was isolated by filtration, washed with diethyl ether (5 ml) and dried in vacuo. The title compound (0.24 g) was further purified by flash chromatography [10 g of silica gel, eluant: ethyl acetate/methanol=9:1 (v/v)] and was obtained in 13% yield (0.14 g of a foamy solid).

¹H-NMR (dmso-d₆, 200 MHz): δ=2.16, 2.25, 2.35 (m_{c}, 2 s, 8H), 2.73 (bs, overlay with dmso signal), 3.20 (bs, overlay with water signal), 3.58 (bs, 2H), 5.27 (dd, 1H), 7.43 (m_{c}, 6H), 7.76 (s, 1H).

### 19. 2,3-Dimethyl-9-phenyl-7H-8,9-dihydro-pyrano[2,3-c]-imidazo[1,2-a]pyridine-6-carboxylic acid methoxy-methyl-amide

A suspension of 2,3-dimethyl-9-phenyl-7H-8,9-dihydro-pyrano[2,3-c]-imidazo[1,2-a]pyridin-6-carboxylic acid (example xiii, 0.400 g, 1.24 mmol) and TBTU (0.470 g, 1.46 mmol) in dichloromethane (20 ml) was heated at reflux for a period of 2 hours. N,O Dimethylhydroxylamine hydrochloride (150 mg, 1.54 mmol) was treated with saturated sodium bicarbonate solution (3 ml) and diethyl ether (3 ml) and the phases were separated. At room temperature, the etherous phase (containing N,O dimethylhydroxylamine) was added to the reaction mixture. The suspension was stirred for 2 hours at room temperature. The same amount of N,O-dimethylhydroxylamine was added and the reaction mixture was heated for 2 hours at 50° C. Another equivalent of the reagent was added and the reaction was continued for 2 hours at 50° C. The brown suspension was cooled to 0° C. and was poured onto a stirred mixture of sodium bicarbonate solution (20 ml) and dichloromethane (25 ml). Stirring was continued for several minutes, the phases were separated, and the aqueous phase was extracted with dichloromethane (3×8 ml). The combined organic phases were washed with water (2×10 ml), dried over sodium sulfate, and concentrated in vacuo. The residue (380 mg of a brown oil) was purified by flash chromatography [15 g of silica gel, eluant: dichloromethane/methanol=100:3 (v/v)]. The corresponding fractions were evaporated and the residue (200 mg) was crystallized from acetone (0.5 ml) and diethyl ether (4 ml). The title compound was isolated by filtration, washed with a few drops of cold acetone and with diethyl ether (3 ml), and dried in vacuo. This afforded 150 mg of a colourless solid (33% yield).

Melting point: 190-192° C. (acetone/diethyl ether)

¹H-NMR (DMSO-d6, 200 MHz): δ=2.12 (mc, 1H), 2.26 (mc, s, 4H), 2.36 (s, 3H), 2.54 (mc), 2.81 (mc, 1H), 3.26 (s, 3H), 3.57 (s, 3H), 5.26 (dd, 1H), 7.42 (mc, 5H), 7.92 (s, 1H).

### 20. 6-(4,5-Dihydro-oxazol-2-yl)-2,3-dimethyl-9-phenyl-7H-8,9-dihydro-pyrano[2,3-c]-imidazo[1,2-a]pyridine

Three samples of 2,3-dimethyl-9-phenyl-7H-8,9-dihydro-pyrano[2,3-c]imidazo[1,2-a]pyridine-6-carboxylic acid (2-chloroethyl)-amide (example xv, 3×70 mg, 0.55 mmol) were transferred into microwave tubes and dissolved in dry DMF (3×3 ml). The yellow solution was heated to 150° C. for 20 minutes and to 170° C. for another 20 minutes. The reaction mixtures were combined and evaporated to dryness. The residue was purified by flash chromatography [22 g of silica gel, eluant: ethyl acetate/methanol=100:3 (v/v)]. Evaporation of the corresponding fractions furnished a red solid (106 mg, mixture of title compound with untransformed starting material as indicated by TLC analysis), which was further purified by preparative HPLC. The title compound was isolated in 14% yield (27 mg of a colourless solid).

¹H-NMR (CDCl₃, 200 MHz): δ=2.30, 2.40, 2.41 (m, 2 s, 8H), 3.15 (m_{c}, 2H), 4.09 (m_{c}, 2H), 4.38 (m_{c}, 2H), 5.30 (dd, 1H), 7.39 (m_{c}, 5H), 8.09 (s, 1H).

### 21. 6-Cyano-2,3-dimethyl-9-phenyl-7H-8,9-dihydro-pyrano[2,3-c]-imidazo[1,2-a]pyridine

A suspension of 2,3-dimethyl-9-phenyl-7H-8,9-dihydro-pyrano[2,3-c]imidazo[1,2-a]pyridine-6-carboxylic acid amide (example xvi, 200 mg, 0.62 mmol) in dry acetonitrile (10 ml) was treated with sodium azide (590 mg, 9.08 mmol) and tetrachlorosilane (0.35 ml, 0.52 g, 3.0 mmol). The white suspension was heated at 95° C. for 3 days. The reaction mixture was cooled and poured onto saturated sodium bicarbonate solution (3 ml), water (15 ml), and dichloromethane (20 ml). The phases were separated and the aqueous phase was extracted with dichloromethane (2×5 ml). The combined organic phases were washed with water (5 ml), dried over sodium sulfate, and evaporated to dryness. The yellow-brown residue (130 mg) was purified by flash chromatography (10 g of silica gel, eluant: dichloromethane/methanol=20:1 (v/v)]. The corresponding fractions were evaporated and the residue was dried in vacuo. This afforded the pure title compound (90 mg of a slightly brown solid, 49% yield).

Melting point: 266-268° C.

¹H-NMR (CDCl₃, 200 MHz): δ=2.18 (m_{c}, 1H), 2.27 (m_{c}, s, 4H), 2.39 (s, 3H), 2.74 (m_{c}, 1H), 3.00 (m_{c}, 1H), 5.27 (dd, 1H), 7.42 (m_{c}, 5H), 8.65 (s, 1H).

II. Compounds of the Formula 1-a

### A. (9S)-3-Acetyl-2-methyl-9-phenyl-7H-8,9-dihydro-pyrano[2,3-c]-imidazo[1,2-a]pyridine-6-carboxylic acid dimethylamide

Resolution of racemic 3-acetyl-2-methyl-9-phenyl-7H-8,9-dihydro-pyrano[2,3-c]-imidazo[1,2-a]pyridine-6-carboxylic acid dimethylamide (example 9, 202 mg, 0.54 mmol) was achieved by preparative chromatography using a 250×20 mm CHIRALPAK® AD-H 5 μm column. The mobile phase consisted of a mixture of n-heptane and ethanol [85:15 (v/v)]. The separation was performed at room temperature with a flow rate of 20 ml/min. The products were detected at a wavelength of 300 nm. The second-eluting enantiomer was identified as the title compound ((9S)-enantiomer) (97 mg, 48% yield, 99.4% ee).

Melting point: 261° C.

The set-up of the analytical method for the HPLC determination of the optical purity was as follows: column: 250×4.6 mm CHIRALPAK® AD 10 μm; mobile phase: n-heptane/ethanol [85:15 (v/v)]; flow rate: 1.5 ml/min; 35° C. The title compound (detection at 220 nm) was eluted after 16.66 min (99.4% ee).

Determination of the optical purity by CE: RT=14.9 min/99.4% ee.

Optical rotation: [α]^{D}₂₀=−30° (c=0.46, chloroform).

¹H-NMR (dmso-d₆, 200 MHz): δ=2.24 (m_{c}, 2H), 2.57 (s, m_{c}, 4H), 2.69 (s, 3H), 2.86 (s, m_{c}, 4H), 3.03 (s, 3H), 5.35 (dd, 1H), 7.44 (m, 5H), 9.10 (s, 1H).

### B. (9S)-2,3-Dimethyl-9-phenyl-7H-8,9-dihydro-pyrano[2,3-c]-imidazo[1,2-a]pyridine-6-carboxylic acid cyclopropylamide

Resolution of racemic 2,3-dimethyl-9-phenyl-7H-8,9-dihydro-pyrano[2,3-c]-imidazo[1,2-a]pyridine-6-carboxylic acid cyclopropylamide (example 10, 207 mg, 0.57 mmol) was achieved by preparative chromatography using a 250×20 mm CHIRALPAK® AD-H 5 μm column. The mobile phase consisted of a mixture of n-heptane and ethanol [85:15 (v/v)]. The separation was performed at room temperature with a flow rate of 20 ml/min. The products were detected at a wavelength of 300 nm. The second-eluting enantiomer was identified as the title compound ((9S)-enantiomer) (100 mg, 48% yield, 99.0-99.5% ee, sample contained 10 weight-% of ethanol).

Melting point: 273° C.

The set-up of the analytical method for the HPLC determination of the optical purity was as follows: column: 250×4.6 mm CHIRALPAK® AD 10 μm; mobile phase: n-heptane/ethanol [85:15 (v/v)]; flow rate: 1.0 ml/min; 25° C. The title compound (detection at 220 nm) was eluted after 8.14 min (99.5% ee).

Determination of the optical purity by CE: RT=16.3 min/99.0% ee.

Optical rotation: [α]^{D}₂₀=−50° (c=0.56, chloroform).

¹H-NMR (dmso-d₆, 200 MHz): δ=0.57 (m, 2H), 0.70 (m_{c}, 2H), 1.06 (t, EtOH), 2.06 (m_{c}, 1H), 2.26 (s, m_{c}, 4H), 2.37 (s, 3H), 2.66-3.08 (m, 3H), 3.44 (dq, EtOH), 4.32 (t, EtOH), 5.23 (dd, 1H), 7.42 (m_{c}, 5H), 7.86 (s, 1H), 8.42 (d, 1H).

C. (9S)-(2,3-Dimethyl-9-phenyl-7H-8,9-dihydro-pyrano[2,34-imidazo[1,2-a]pyridin-6-yl)-azetidin-1-yl methanone

Resolution of racemic (2,3-dimethyl-9-phenyl-7H-8,9-dihydro-pyrano[2,3-c]-imidazo[1,2-a]pyridin-6-yl)-azetidin-1-yl methanone (example 11, 209 mg, 0.58 mmol) was achieved by preparative chromatography using a 250×50 mm CHIRALPAK® 50801 20 μm column. Ethanol was used as mobile phase. The separation was performed at room temperature with a flow rate of 120 ml/min. The products were detected at a wavelength of 300 nm. The first-eluting enantiomer was identified as the title compound ((9S)-enantiomer) (100 mg, 48% yield, 100% ee).

Melting point: 248° C.

The set-up of the analytical method for the HPLC determination of the optical purity was as follows: column: 250×4.6 mm CHIRALPAK® 50801 20 μm; mobile phase: ethanol; flow rate: 1.0 ml/min; 30° C. The title compound (detection at 220 nm) was eluted after 11.48 min (100% ee).

Determination of the optical purity by CE: RT=14.8 min/100% ee.

Optical rotation: [α]^{D}₂₀=−50° (c=0.50, chloroform).

¹H-NMR (dmso-d₆, 200 MHz): δ=2.12, 2.25 (m_{c}, s, 7H), 2.37 (s, 3H), 2.66 (m_{c}, 1H), 2.92 (m_{c}, 1H), 4.06 (m, 4H), 5.25 (dd, 1H), 7.42 (m_{c}, 5H), 7.86 (s, 1H).

D. (9S)-2,3-Dimethyl-9-phenyl-7H-8,9-dihydro-pyrano[2,3-c]-imidazo[1,2-a]pyridine-6-carbothioic acid dimethylamide

A suspension of (9S)-2,3-dimethyl-9-phenyl-7H-8,9-dihydro-pyrano[2,3-c]-imidazo[1,2-a]pyridine-6-carboxylic acid dimethylamide (800 mg, 2.29 mmol) and Lawesson reagent (1.10 g, 2.7 mmol) in 1,2-dimethoxyethane (20 ml) was heated to 50° C. After the reaction temperature had been reached, the mixture was diluted with more 1,2-dimethoxyethane (15 ml) and stirring was continued for 2 hours at 50° C. The reaction was cooled and poured onto a mixture of saturated bicarbonate solution (25 ml) and dichloromethane (60 ml). The phases were separated and the aqueous phase was extracted with dichloromethane (2×10 ml). The combined organic phases were washed with water (2×20 ml), dried over sodium sulfate and concentrated under reduced pressure. The residue (1.8 g of a yellow oil) was purified by flash chromatography [15 g of silica gel, eluant: dichloromethane/methanol=100:4 (v/v)]. Evaporation of the corresponding fractions afforded an oily residue (750 mg), which was crystallized from acetone (1 ml). After a period of 1 hour, the precipitate was isolated by filtration, washed with acetone (0.5 ml) and diethyl ether (5 ml), and dried in vacuo. The title compound was obtained in the form of a colourless solid (44% yield).

Melting point: 244-245° C. (acetone/diethyl ether)

¹H-NMR (DMSO-d6, 200 MHz): =2.12 (mc, 1H), 2.26, 2.29, 2.34 (s, mc, s, 7H), 2.63 (mc, 1H), 2.85 (mc, 1H), 3.05, 3.17 (2 s, 3H), 3.51, 3.52 (2 s, 3H), 5.26 (mc, 1H), 7.41 (mc, 5H), 7.64, 7.65 (2 s, 1H).

III. Compounds of the Formula 1-b

### a. (9R)-3-Acetyl-2-methyl-9-phenyl-7H-8,9-dihydro-pyrano[2,3-c]-imidazo[1,2-a]pyridine-6-carboxylic acid dimethylamide

Resolution of racemic 3-acetyl-2-methyl-9-phenyl-7H-8,9-dihydro-pyrano[2,3-c]-imidazo[1,2-a]pyridine-6-carboxylic acid dimethylamide (example 9, 202 mg, 0.54 mmol) was performed as described in example A. The first-eluting enantiomer was identified as the title compound ((9R)-enantiomer) (97 mg, 48% yield, 99.4-99.6% ee).

Melting point: 260° C.

The set-up of the analytical method for the HPLC determination of the optical purity is described in example A. The title compound (detection at 220 nm) was eluted after 14.38 min (99.6% ee).

Determination of the optical purity by CE: RT=15.3 min/99.4% ee.

Optical rotation: [α]^{D}₂₀=25° (c=0.46, chloroform).

¹H-NMR (dmso-d₆, 200 MHz): δ=2.24 (m_{c}, 2H), 2.57 (s, m_{c}, 4H), 2.69 (s, 3H), 2.86 (s, m_{c}, 4H), 3.03 (s, 3H), 5.35 (dd, 1H), 7.44 (m_{c}, 5H), 9.10 (s, 1H).

### b. (9R)-2,3-Dimethyl-9-phenyl-7H-8,9-dihydro-pyrano[2,3-c]-imidazo[1,2-a]pyridine-6-carboxylic acid cyclopropylamide

Resolution of racemic 2,3-dimethyl-9-phenyl-7H-8,9-dihydro-pyrano[2,3-c]-imidazo[1,2-a]pyridine-6-carboxylic acid cyclopropylamide (example 10, 207 mg, 0.57 mmol) was performed as described in example B. The first-eluting enantiomer was identified as the title compound ((9R)-enantiomer) (100 mg, 48% yield, 99.2-99.4% ee, sample contained 10 weight-% of ethanol).

Melting point: 270° C.

The set-up of the analytical method for the HPLC determination of the optical purity is described in example B. The title compound (detection at 220 nm) was eluted after 6.54 min (99.2% ee).

Determination of the optical purity by CE: RT=17.0 min/99.4% ee.

Optical rotation: [α]^{D}₂₀₌₃₅° (c=0.44, chloroform).

¹H-NMR (dmso-d₆, 200 MHz): δ=0.57 (m_{c}, 2H), 0.70 (m_{c}, 2H), 1.06 (t, EtOH), 2.06 (m_{c}, 1H), 2.26 (s, m_{c}, 4H), 2.37 (s, 3H), 2.66-3.08 (m, 3H), 3.44 (dq, EtOH), 4.32 (t, EtOH), 5.23 (dd, 1H), 7.42 (m_{c}, 5H), 7.86 (s, 1H), 8.42 (d, 1H).

### c. (9R)-(2,3-Dimethyl-9-phenyl-7H-8,9-dihydro-pyrano[2,3-c]-imidazo[1,2-a]pyridin-6-yl)-azetidin-1-yl methanone

Resolution of racemic (2,3-dimethyl-9-phenyl-7H-8,9-dihydro-pyrano[2,3-c]-imidazo[1,2-a]pyridin-6-yl)-azetidin-1-yl methanone (example 11, 209 mg, 0.58 mmol) was performed as described in example C. The second-eluting enantiomer was identified as the title compound ((9R)-enantiomer) (100 mg, 48% yield, 99.6% ee).

Melting point: 247° C.

The set-up of the analytical method for the HPLC determination of the optical purity is described in example C. The title compound (detection at 220 nm) was eluted after 18.93 min (99.6% ee).

Determination of the optical purity by CE: RT=15.2 min/99.6% ee.

Optical rotation: [α]_{D}²⁰−26° (c=0.50, chloroform).

¹H-NMR (dmso-d₆, 200 MHz): δ=2.12, 2.25 (m_{c}, s, 7H), 2.37 (s, 3H), 2.66 (m_{c}, 1H), 2.92 (m_{c}, 1H), 4.06 (m_{c}, 4H), 5.25 (dd, 1H), 7.42 (m_{c}, 5H), 7.86 (s, 1H).

IV. Starting Compounds and Intermediates

### Synthesis of Intermediates for Racemic 7H-8,9-dihydro-pyrano[2,3-c]-imidazo[1,2-a]pyridines Via Cross Metathesis

### i. 2-Amino-3-benzyloxy-5-bromo-pyridine

2-Amino-3-benzyloxypyridine (85.0 g, 0.42 mol) was dissolved in a 10% aqueous solution of sulphuric acid (1000 ml). The yellow solution was cooled to 0 to 4° C. and a solution of bromine (80.5 g, 0.50 mol) in acetic acid (276 g, 4.6 mol) was added dropwise over a period of 2 h. A red suspension was obtained which was stirred for 2.5 h at 0° C. and was then poured onto a mixture of ice water (500 ml) and dichloromethane (1000 ml). A pH-value of 8 was adjusted by addition of 25% aqueous ammonia solution (approx. 600 ml) to the well-stirred biphasic mixture. The phases were separated and the aqueous phase was extracted with dichloromethane (3×500 ml). The combined organic phases were washed with water (400 ml) and dried over sodium sulfate. The solvent was removed under reduced pressure and the residue was purified by flash chromatography [1 kg of silica gel, eluant: petrol ether/ethyl acetate=7:3 (v/v)]. Thus, 96.0 g of the title compound were isolated in form of a brown solid (81% yield).

Melting point: 109-110° C.

¹H-NMR (CDCl₃, 200 MHz): δ=4.73 (bs, 2H), 5.04 (s, 2H), 7.08 (d, 1H), 7.40 (m_{c}, 5H), 7.73 (d, 1H).

### ii. 8-Benzyloxy-6-bromo-2-methyl-imidazo[1,2-a]pyridine

A well-stirred solution of 2-amino-3-benzyloxy-5-bromo-pyridine (96.0 g, 0.34 mol) and chloroacetone (50 ml, 58.0 g, 0.63 mol) in dry THF (300 ml) was heated to 60° C. After 3.5 days, the precipitate formed in the course of the reaction was removed by filtration, washed with THF (30 ml), and dried in vacuo. The mother liquor was treated with more chloroacetone (50 ml, 58.0 g, 0.63 mol) and the reaction mixture was stirred at 60° C. for another 8 days. More precipitate was formed which was again isolated by filtration, washed with THF (30 ml), and dried in vacuo. The two crops (55+48 g), were combined and were crystallized from hot isopropanol (800 ml). The obtained colourless crystals (55 g) were dissolved in a biphasic mixture of water and dichloromethane. The mixture was neutralized by addition of a 6 N aqueous solution of sodium hydroxide. The phases were separated and the aqueous phase was extracted with dichloromethane (2×50 ml). The combined organic phases were dried over sodium sulfate and concentrated under reduced pressure. The obtained solid was purified by flash chromatography [1.7 kg of silica gel, eluant: petrol ether/ethyl acetate=8:2 (v/v)]. The mother liquor of the crystallization step was concentrated and the residue (48 g) was purified as described above. A total amount of 63.7 g (59% yield) of a sticky yellow solid was isolated, which was the pure title compound as indicated by ¹H-NMR analysis.

¹H-NMR (CDCl₃, 200 MHz): δ=2.43 (s, 3H), 5.28 (s, 2H), 6.52 (d, 1H), 7.37 (m_{c}, 6H), 7.79 (d, 1H).

### iii. 8-Benzyloxy-2-methyl-imidazo[1,2-a]pyridine-6-carboxylic acid dimethylamide

A solution of 8-benzyloxy-6-bromo-2-methyl-imidazo[1,2-a]pyridine (146.0 g, 0.46 mol) in dry THF (31) was transferred into an autoclave. After addition of palladium acetate (11.5 g, 0.05 mol), triphenylphosphine (71.0 g, 0.27 mol), triethylamine (132 ml, 0.94 mol), and a 2 M solution of dimethylamine in THF (1.2 l, 2.4 mol), the autoclave was pressurized with carbon monoxide (6 bar) and was heated to 120° C. After a reaction time of 18 hours the reaction mixture was cooled, filtered, and concentrated in vacuo. The residue was dissolved in dichloromethane (700 ml) and water (300 ml). The phases were separated and the aqueous phase was extracted with dichloromethane (100 ml). The combined organic phases were dried over sodium sulfate and concentrated under reduced pressure. A sticky brown residue (219 g) remained which was purified by flash chromatography (4.4 kg of silica gel, eluant: ethyl acetate, then ethyl acetate/methanol=9:1). The title compound was isolated as a beige solid (110 g, 77% yield), pure by means of ¹H-NMR spectroscopy.

¹H-NMR (CDCl₃, 200 MHz): δ=2.47 (s, 3H), 2.95 (bs, 6H), 5.35 (s, 2H), 6.43 (d, 1H), 7.40 (m_{c}, 6H), 7.88 (d, 1H).

### iv. 8-Hydroxy-2-methyl-imidazo[1,2-a]pyridine-6-carboxylic acid dimethylamide

A solution of 8-benzyloxy-2-methyl-4-imidazo[1,2-a]pyridine-6-carboxylic acid dimethylamide (58.0 g, 0.19 mol) in methanol (500 ml) was treated with the hydrogenation catalyst (10% Palladium on charcoal, 7 g) and a hydrogen pressure of 1 bar was applied. After the suspension had been stirred for 18 hours at room temperature, the catalyst was removed by filtration and the filtrate was concentrated in vacuo. The title compound (40.1 g, 98% yield) was isolated as a beige solid.

¹H-NMR (CDCl₃, 200 MHz): δ=2.44 (s, 3H), 3.10 (bs, 6H), 6.74 (d, 1H), 7.31 (s, 1H), 7.89 (d, 1H), 8.96 (bs, 1H).

### v. 8-Allyloxy-2-methyl-imidazo[1,2-a]pyridine-6-carboxylic acid dimethylamide

The alcohol 8-hydroxy-2-methyl-imidazo[1,2-a]pyridine-6-carboxylic acid dimethylamide (4.74 g, 21.6 mmol) was dissolved in dry DMF (50 ml). Potassium carbonate (2.98 g, 21.6 mmol) and allyl bromide (3.14 g, 25.9 mmol) was added and the reaction mixture was stirred at room temperature for 18.5 hours. The solvent was removed under reduced pressure and the residue was dissolved in saturated ammonium chloride solution (100 ml) and chloroform (150 ml). The phases were separated and the aqueous phase was extracted with chloroform (2×150 ml). The combined organic phases were dried over sodium sulfate and concentrated under reduced pressure. The obtained dark-brown liquid (8.5 g) was purified by flash chromatography [250 g of silica gel, eluant: ethyl acetate/methanol=4:1 (v/v)]. The title compound was isolated in 70% yield (5.05 g) in form of a yellowish oil. Traces of impurities (approximately 5 mol-%) were visible in the ¹H-NMR spectrum.

¹H-NMR (CDCl₃, 200 MHz): δ=2.46 (s, 3H), 3.09 (s, 6H), 4.79 (dt, 2H), 5.33 (dd, 1H), 5.45 (dd, 1H), 6.15 (ddt, 1H), 6.48 (d, 1H), 7.33 (s, 1H), 7.87 (d, 1H).

### vi. 7-Allyl-8-hydroxy-2-methyl-imidazo[1,2-a]pyridine-6-carboxylic acid dimethylamide

A flask containing neat 8-allyloxy-2-methyl-imidazo[1,2-a]pyridine-6-carboxylic acid dimethylamide (3.93 g, 15.2 mmol) was put into an oil-bath, which had been pre-heated to 160° C. After a period of 50 minutes at 160° C., the reaction mixture solidified forming a dark brown solid. The crude product was cooled to room temperature and was treated with a mixture of acetone and diethyl ether [1:1 (v/v), 20 ml]. A colourless solid precipitated, which was removed by filtration, washed with diethyl ether (10 ml), and dried in vacuo. Thus, 2.10 g of the pure title compound were isolated. The mother liquor was concentrated under reduced pressure and purified by flash chromatography (70 g of silica gel, eluant: ethyl acetate/methanol=9:1 then 4:1 (v/v)] yielding another 0.48 g of the title compound (2.58 g, 66% overall yield).

¹H-NMR (CDCl₃, 200 MHz): δ=2.43 (s, 3H), 2.88 (s, 3H), 3.11 (s, 3H), 3.55 (bd, 2H), 5.00, 5.07 (2 dd, 2H), 5.98 (m_{c}, 1H), 7.22 (s, 1H), 7.53 (s, 1H), 9.57 (bs, 1H).

### vii. Pivaloic acid [7-allyl-6-dimethylcarbamoyl-2-methyl-imidazo[1,2-a]pyridin-8-yl]ester

To a suspension of 7-allyl-8-hydroxy-2-methyl-imidazo[1,2-a]pyridine-6-carboxylic acid dimethylamide (1.00 g, 3.9 mmol) in acetone (30 ml), potassium carbonate (0.53 g, 3.9 mmol) and pivaloyl chloride (0.93 g, 7.7 mmol) was added. The yellow suspension was stirred for 3 hours at room temperature. After addition of saturated ammonium chloride solution (20 ml) and water (10 ml) the reaction mixture was extracted with dichloromethane (3×50 ml). The combined organic phases were dried over sodium sulfate and concentrated under reduced pressure. The crude product (1.46 g of a colourless solid) was purified by flash chromatography (30 g of silica gel, eluant: ethyl acetate). The title compound was obtained in 72% yield (0.96 g).

Melting point: 178-180° C.

¹H-NMR (CDCl₃, 200 MHz): δ=1.48 (s, 9H), 2.41 (s, 3H), 2.89 (s, 3H), 3.08 (s, 3H), 3.35 (d, 2H), 5.04 (m_{c}, 2H), 5.78 (m_{c}, 1H), 7.28 (s, 1H), 7.82 (s, 1H).

### viii. (E)-Pivaloic acid [6-dimethylcarbamoyl-2-methyl-7-(3-phenyl-allyl)-imidazo[1,2-a]pyridin-8-yl]ester

Pivaloic acid [7-allyl-6-dimethylcarbamoyl-2-methyl-imidazo[1,2-a]pyridin-8-yl]ester (9.30 g, 27.1 mmol) was dissolved in dichloromethane (140 ml), which had been degassed with argon. After addition of trans-stilbene (19.53 g, 108.4 mmol) and second-generation Grubbs catalyst (CAS 246047-72-3, 920 mg, 1.08 mmol, 4 mol-%) a red solution was obtained. The reaction mixture was heated to 40° C. and was stirred for 18 hours at this temperature. The crude product obtained on concentration of the green solution was purified by flash chromatography [1.2 kg of silica gel, eluant: petrolether (to remove excess trans-stilbene), then ethyl acetate]. A slightly green solid (6.6 g) was isolated which consisted of the title compound (90 mol-%, 53% yield) and untransformed starting material (10 mol-%, ratio determined by ¹H-NMR analysis).

¹H-NMR data of the title compound, derived from a 9:1 mixture with untransformed starting material (CDCl₃, 200 MHz): δ=1.49 (s, 9H), 2.42 (s, 3H), 2.79 (s, 3H), 3.01 (s, 3H), 3.53 (d, 2H), 6.12 (dt, 1H), 6.43 (d, 1H), 7.24 (m_{c}, 6H), 7.81 (s, 1H). The NMR-signals of the starting material are reported above.

### ix. 2-Methyl-9-phenyl-7H-8,9-dihydro-pyrano[2,3-c]imidazo[1,2-a]pyridine-6-carboxylic acid dimethylamide

The product of the cross-metathesis reaction (example viii, 6.6 g), containing (E)-pivaloic acid [6-dimethylcarbamoyl-2-methyl-7-(3-phenyl-allyl)-imidazo[1,2-a]pyridin-8-yl]ester (6.05 g, 14.4 mmol) and pivaloic acid [7-allyl-6-dimethylcarbamoyl-2-methyl-imidazo[1,2-a]pyridin-8-yl]ester (0.55 g, 1.6 mmol) was treated with 200 ml of orthophosphoric acid (85%). The resulting green solution was heated for 50 minutes to 80° C. The reaction mixture was cooled to room temperature, diluted with dichloromethane (200 ml), and neutralized with a 6 N solution of sodium hydroxide at 0° C. The phases were separated and the aqueous phase was extracted with dichloromethane (2×200 ml). The combined organic phases were dried over sodium sulfate and concentrated under reduced pressure. The crude product was purified by flash chromatography [210 g of silica gel, eluant: ethyl acetate/methanol=9:1 (v/v)]. A colourless solid (4.4 g, 91% yield) was obtained, which was the pure title compound as indicated by ¹H-NMR analysis.

Melting point: 189° C.

¹H-NMR (CDCl₃, 200 MHz): δ=2.26 (m_{c}, 2H), 2.41 (s, 3H), 2.58, 2.77 (2 m_{c}, 2H), 2.94 (s, 3H), 3.12 (s, 3H), 5.31 (dd, 1H), 7.40 (m_{c}, 6H), 7.67 (s, 1H).

### x. 2-Methyl-9-phenyl-7H-8,9-dihydro-pyrano[2,3-c]imidazo[1,2-a]pyridine-6-carboxylic acid dimethylamide Prepared by One-Pot Synthesis

The title compound can also be obtained by application of a one-pot procedure: In a flame-dried flask filled with argon, pivaloic acid [7-allyl-6-dimethylcarbamoyl-2-methyl-imidazo[1,2-a]pyridin-8-yl]ester (example vii, 4.80 g, 14.0 mmol) was dissolved in dichloromethane (100 ml) which had been degassed with argon. After addition of trans-stilbene (10.10 g, 56.0 mmol) and second-generation Grubbs catalyst (CAS 246047-72-3, 475 mg, 0.56 mmol, 4 mol-%) the solution was heated to 40° C. The reaction mixture was stirred for 18 hours at this temperature and was then concentrated under reduced pressure. A green solid was obtained which was treated with 100 ml of orthophosphoric acid (85%). The suspension was heated to 80° C. After a period of 1 hour, a clear solution was obtained which was cooled to room temperature and poured onto a mixture of ice water (50 ml) and dichloromethane (50 ml). A pH-value of 8 was adjusted by addition of 6 N sodium hydroxide solution. The phases were separated and the aqueous phase was extracted with dichloromethane (2×20 ml). The combined organic phases were dried over sodium sulfate and concentrated under reduced pressure. The residue, 16 g of a green solid, was purified by flash chromatography [320 g of silica gel, eluant: petrol ether (to remove excess trans-stilbene), then ethyl acetate/methanol=100:2 (v/v)]. The title compound (3.0 g, 64% yield) was isolated as a green foamy solid, pure by means of ¹H-NMR spectroscopy.

¹H-NMR (CDCl₃, 200 MHz): δ=2.26 (m_{c}, 2H), 2.41 (s, 3H), 2.58, 2.77 (2 m_{c}, 2H), 2.94 (s, 3H), 3.12 (s, 3H), 5.31 (dd, 1H), 7.40 (m, 6H), 7.67 (s, 1H).

### xi. 3-Formyl-2-methyl-9-phenyl-7H-8,9-dihydro-pyrano[2,3-c]imidazo[1,2-a]pyridine-6-carboxylic acid dimethylamide

A flask containing dry DMF (12 ml) was cooled to 0° C. and phosphorus oxychloride (0.914 g, 5.96 mmol) was added. The cooling bath was removed and the solution was stirred for 1 hour at mom temperature. The red reaction mixture was treated with a solution of 2-methyl-9-phenyl-7H-8,9-dihydro-pyrano[2,3-c]imidazo[1,2-a]pyridine-6-carboxylic acid dimethylamide (0.800 g, 2.39 mmol) in dry DMF (12 ml) and was heated to 60° C. After a period of 5 hours, the reaction mixture was poured on ice water (10 ml), neutralized by addition of 6 N sodium hydroxide solution, and then extracted with dichloromethane (3×20 ml). The combined organic phases were dried over sodium sulfate and concentrated in vacuo. The title compound (0.700 g, 81% yield) was obtained as a yellow solid, pure by means of ¹H-NMR spectroscopy.

¹H-NMR (CDCl₃, 200 MHz): δ=2.31 (m_{c}, 2H), 2.72 (s, m_{c}, 4H), 2.89, 2.95 (m_{c}, s, 4H), 3.15 (s, 3H), 5.34 (dd, 1H), 7.39 (m_{c}, 5H), 9.09 (s, 1H), 9.99 (s, 1H).

### xii. 2-Methyl-3-nitroso-9-phenyl-7H-8,9-dihydro-pyrano[2,3-c]imidazo[1,2-a]pyridine-6-carboxylic acid dimethylamide

In a flame-dried flask filled with argon, a solution of 2-methyl-9-phenyl-7H-8,9-dihydro-pyrano[2,3-c]imidazo[1,2-a]pyridine-6-carboxylic acid dimethylamide (example 1x, 0.70 g, 2.1 mmol) in dry THF (15 ml) was treated with isopentyl nitrite (2.44 g, 20.8 mmol). The reaction mixture was stirred for 2.5 hours at 40° C. and was then concentrated in vacuo. The dark crude product was purified by flash chromatography (16 g of silica gel, eluant: ethyl acetate). Evaporation of the corresponding fractions yielded the title compound in the form of a green, foamy solid (0.56 g, 74% yield).

¹H-NMR (CDCl₃, 200 MHz): δ=2.32 (m_{c}, 2H), 2.83, 2.92 (m_{c}, s, 5H), 3.15, 3.16 (2 s, 6H), 5.37 (dd, 1H), 7.39 (m_{c}, 5H), 9.37 (s, 1H), additional signals at 7.10 (d) and 7.94 (d).

### Synthesis of Intermediates for Racemic 7H-8,9-dihydro-pyrano[2,3-c]-imidazo[1,2-]pyridines via saponification of ethyl 2,3-dimethyl-9-phenyl-7H-8,9-dihydro-pyrano[2,3-c]-imidazo[1,2-a]pyridine-6-carboxylic acid

### xiii. 2,3-Dimethyl-9-phenyl-7H-8,9-dihydro-pyrano[2,3-c]-imidazo[1,2-a]pyridine-6-carboxylic acid

A suspension of ethyl 2,3-dimethyl-9-phenyl-7H-8,9-dihydro-pyrano[2,3-c]-imidazo[1,2-a]pyridine-6-carboxylate (synthesis described in WO 03/014123, 16.7 g, 48 mmol) in methanol (170 ml) and water (35 ml) was treated with potassium hydroxide (4.5 g, 80 mmol) and was heated to 50° C. After a reaction time of 2 hours, the methanol was removed in vacuo. Water (400 ml) and dichloromethane (300 ml) was added, a pH-value of 4.8 (isoelectric point of the title compound) was adjusted by addition of 6 N hydrochloric acid, and stirring was continued for 30 minutes. A precipitate was formed, which slowly dissolved after addition of dichloromethane (100 ml) and methanol (100 ml). The phases were separated and the aqueous phase was extracted with dichloromethane (2×50 ml). The combined organic phases were dried over sodium sulfate and concentrated to a volume of 50 ml. Upon addition of diethyl ether (100 ml) a colourless precipitate was formed. Stirring was continued for 30 minutes at 0° C. The precipitate was removed by filtration and dried in vacuo yielding 9.1 g of the pure title compound (58% yield). The aqueous phase was saturated with sodium chloride and extracted with chloroform (1×400 ml, 2×100 ml). The combined organic phases were dried over sodium sulfate and concentrated in vacuo. The residue (2.0 g, 13% yield) was pure title compound as judged by ¹H-NMR spectroscopy.

Melting point: 318-320° C. (diethyl ether)

¹H-NMR (dmso-d₆, 200 MHz): δ=2.09 (m_{c}, 1H), 2.28 (s, m_{c}, 4H), 2.40 (s, 3H), 3.10 (m_{c}, 2H), 5.25 (dd, 1H), 7.43 (m_{c}, 5H), 8.32 (s, 1H), exchangeable protons not visible.

Elemental analysis: calculated for C₁₉H₁₈N₂O₃.(H₂O)_{0.5} (322.37+9.0): C, 68.87; H, 5.78; N, 8.45. found: C, 68.95, H 5.49, N 8.40.

### xiv. 2,3-Dimethyl-9-phenyl-7H-8,9-dihydro-pyrano[2,3-c]imidazo[1,2-a]pyridine-6-carboxylic acid (2-hydroxyethyl)-amide

A mixture of 2,3-dimethyl-9-phenyl-7H-8,9-dihydro-pyrano[2,3-c]-imidazo[1,2-a]pyridine-6-carboxylic acid (0.50 g, 1.6 mmol) and thionyl chloride (0.34 ml, 0.55 g, 4.6 mmol) was diluted with dry dichloro methane (7 ml). The suspension was treated with DBU (0.24 ml, 0.24 g, 1.6 mmol) and was stirred for 24 hours at room temperature. The light-brown reaction mixture was evaporated to dryness and the residue was dissolved in dry dichloromethane (15 ml). The resulting suspension was cooled to 0° C. and a solution of 2-aminoethanol (0.17 ml, 0.17 g, 2.8 mmol) in dichloromethane (5 ml) was added. The reaction mixture was stirred for 2.5 hours at room temperature. The precipitate was removed by filtration. The filtrate was concentrated in vacuo and the brown residue (0.9 g) was purified by flash chromatography [36 g of silica gel, eluant: ethyl acetate/methanol=10:1 (v/v)]. Evaporation of the corresponding fractions yielded the pure title compound (0.25 g of a colourless solid, 44% yield).

¹H-NMR (CDCl₃, 200 MHz): δ=1.92 (m_{c}), 2.27 (m_{c}, s, 4H), 2.41 (s, 3H), 2.68 (m_{c}, 2H), 3.46 (m_{c}, 2H), 3.71 (m_{c}, 2H), 4.97 (dd, 1H), 7.14 (bt, 1H), 7.27 (s), 7.42 (m, 5H).

### xv. 2,3-Dimethyl-9-phenyl-7H-8,9-dihydro-pyrano[2,3-c]imidazo[1,2-a]pyridine-6-carboxylic acid (2-chloroethyl)-amide

A solution of 2,3-dimethyl-9-phenyl-7H-8,9-dihydro-pyrano[2,3-c]imidazo[1,2-a]pyridine-6-carboxylic acid (2-hydroxyethyl)-amide (0.30 g, 0.8 mmol) in thionyl chloride (0.40 ml, 0.65 g, 5.5 mmol) was stirred for 1 hour at room temperature. It was then diluted with dichloromethane (30 ml) and water (5 ml) and a neutral pH-value was adjusted by addition of saturated sodium bicarbonate solution. The phases were separated and the aqueous phase was extracted with dichloromethane (20 ml). The combined organic phases were dried over sodium sulfate, concentrated under reduced pressure, and dried in vacuo. The title compound was obtained in 70% yield (0.22 g of a colourless solid).

¹H-NMR (CDCl₃, 200 MHz): δ=2.14 (m_{c}), 2.32, 2.38 (2 s, m_{c}, 7H), 2.85 (m_{c}, 1H), 3.08 (m_{c}, 1H), 3.75 (s, 4H), 5.21 (dd, 1H), 6.90 (bs, 1H), 7.36 (m_{c}, 5H), 7.60 (s, 1H).

### xvi. 2,3-Dimethyl-9-phenyl-7H-8,9-dihydro-pyrano[2,3-c]-imidazo[1,2-a]pyridine-6-carboxylic acid amide

A suspension of 2,3-dimethyl-9-phenyl-7H-8,9-dihydro-pyrano[2,3-c]-imidazo[1,2-a]pyridine-6-carboxylic acid (example xiii, 500 mg, 1.54 mmol) in dichloromethane (20 ml) was treated with TBTU (504 mg, 1.57 mmol). The reaction mixture was heated for 1 hour at 40° C. and was then allowed to cool to room temperature. Ammonia gas was bubbled through the suspension over a period of 30 minutes. The reaction mixture was poured onto water (20 ml), dichloromethane (30 ml) was added, and a pH-value of 6 was adjusted by addition of 2 N hydrochloric acid. In order to facilitate the separation of the phases, a 10 ml portion of methanol was added. The phases were separated, and the aqueous phase was extracted with dichloromethane (2×10 ml). The combined organic phases were dried over sodium sulfate and concentrated in vacuo. The title compound (310 mg, 64% yield) was isolated in the form of a colourless solid, pure by means of ¹H-NMR spectroscopy.

Melting point: 303-305° C.

¹H-NMR (dmso-d₆, 200 MHz): δ=2.09 (m_{c}, 1H), 2.26 (m_{c}, s, 4H), 2.38 (s, 3H), 2.97 (m_{c}, 2H), 5.24 (dd, 1H), 7.41 (bs, m_{c}, 6H), 7.85 (bs, 1H), 7.98 (s, 1H).

Asymmetric Hydroboration of Prochiral Olefins

### xvii. (E)-8-Hydroxy-2-methyl-7-(3-phenyl-allyl)-imidazo[1,2-a]pyridin-6-carboxylic acid dimethylamide

Pure (E)-Pivaloic acid [6-dimethylcarbamoyl-2-methyl-7-(3-phenyl-allyl)-imidazo[1,2-a]pyridin-8-yl]ester (synthesis as described in example viii) was dissolved in methanol (200 ml). After dropwise addition of a 6N sodium hydroxide solution (12 ml), the reaction mixture was stirred for 1 hour at room temperature and for another hour at 50° C. The dark solution was concentrated to a volume of 30 ml. Water (30 ml) and dichloromethane (50 ml) was added and the biphasic mixture was neutralized by addition of 6 N hydrochloric acid. The phases were separated and the aqueous phase was extracted with dichloromethane (3×15 ml). The combined organic phases were washed with water (20 ml), dried over sodium sulfate, and evaporated to dryness. A dark solid (5 g) was obtained, which was dissolved in a hot mixture of dichloromethane (20 ml) and acetone (60 ml). The stirred solution was allowed to cool to room temperature, at which point crystallization took place. Stirring was continued for 1 hour at room temperature. The precipitate was isolated by filtration, washed with diethyl ether (10 ml) and dried in vacuo. The title compound was isolated in the form of a colourless solid (2.6 g, 55% yield).

Melting point: 188-190° C. (dichloromethane/acetone)

¹H-NMR (dmso-d₆, 200 MHz): δ=2.35 (s, 3H), 2.75 (s, 3H), 2.94 (s, 3H), 3.48 (d, 2H), 6.28 (m_{c}, 2H), 7.26 (m_{c}, 5H), 7.59 (s, 1H), 7.97 (s, 1H).

### viii. (3R)-8-Hydroxy-7-(3-hydroxy-3-phenyl-propyl)-2-methyl-imidazo[1,2-a]pyridine-6-carboxylic acid dimethylamide

A flame-dried flask filled with argon was charged with (R)-Alpine-boramine™ (CAS 67826-92-0, 1.50 g, 3.6 mmol). After addition of dry THF (8 ml) a colourless solution was obtained, which was treated with boron trifluoride diethyl etherate (0.92 ml, 1.03 g, 7.3 mmol). The solution was stirred for 2.5 hours at room temperature and for 1 hour at 0° C. A colourless precipitate was obtained which was removed by filtration and washed with cold THF (6 ml, argon atmosphere). The filtrates [containing (−)-monoisopinocampheylborane] were combined. A suspension of 8-hydroxy-2-methyl-7-(3-phenyl-allyl)imidazo[1,2-a]pyridine-6-carboxylic acid dimethylamide (405 mg, 1.21 mmol) in dry THF (15 ml) was added at room temperature, at which point a yellow solution was obtained. After a reaction time of 2 hours, the solution was slowly added to a cold mixture of aqueous potassium hydroxide solution (230 mg in 1.6 ml of water), ethanol (4 ml), and hydrogen peroxide (30 weight-% in water, 1.6 ml). After a period of 15 minutes, the reaction mixture was poured onto saturated ammonium chloride solution (20 ml) and dichloromethane (40 ml). The phases were separated and the aqueous phase was extracted with dichloromethane (2×10 ml). The combined organic phases were washed with water (10 ml), dried over sodium sulfate, and concentrated under reduced pressure. The crude product (1.7 g of an oil) was purified by flash chromatography [20 g of silica gel, eluant: dichloromethane (to remove isopinocampheol), then dichloromethane/methanol=20:1 (v/v)]. Evaporation of the corresponding fractions furnished a solid (220 mg), which was washed with acetone (1 ml), isolated by filtration, and dried in vacuo. The title compound was isolated in 18% yield (75 mg of a colourless solid, optical purity: 32.2% ee).

Melting point: 223-224° C. (acetone)

Determination of the optical purity by CE: RT [(3S)-enantiomer]=17.6 min/33.2 area-%; RT [(3R)-enantiomer]=17.8 min/64.8 area-%; 32.2% ee (A).

¹H-NMR (dmso-d₆, 200 MHz): δ=1.81 (m_{c}, 2H), 2.33 (s, m_{c}, 4H), 2.65 (m_{c}), 2.77, 2.89 (2 s, 6H), 4.50 (t, 1H), 7.25 (m_{c}, 5H), 7.55 (s, 1H), 7.88 (s, 1H).

Commercial Utility

The compounds of the formula 1 and their salts have valuable pharmacological properties which make them commercially utilizable. In particular, they exhibit marked inhibition of gastric acid secretion and an excellent gastric and intestinal protective action in warm-blooded animals, in particular humans. In this connection, the compounds according to the invention are distinguished by a high selectivity of action, an advantageous duration of action, a particularly good enteral activity, the absence of significant side effects and a large therapeutic range.

“Gastric and intestinal protection” in this connection is understood as meaning the prevention and treatment of gastrointestinal diseases, in particular of gastrointestinal inflammatory diseases and lesions (such as, for example, gastric ulcer, peptic ulcer, including peptic ulcer bleeding, duodenal ulcer, gastritis, hyperacidic or medicament-related functional dyspepsia), which can be caused, for example, by microorganisms (e.g. Helicobacter pylori), bacterial toxins, medicaments (e.g. certain antiinflammatories and antirheumatics, such as NSAIDs and COX-inhibitors), chemicals (e.g. ethanol), gastric acid or stress situations. “Gastric and intestinal protection” is understood to include, according to general knowledge, gastroesophageal reflux disease (GERD), the symptoms of which include, but are not limited to, heartburn and/or acid regurgitation.

In their excellent properties, the compounds according to the invention surprisingly prove to be clearly superior to the compounds known from the prior art in various models in which the antiulcerogenic and the antisecretory properties are determined. On account of these properties, the compounds of the formula 1 and their pharmacologically acceptable salts are outstandingly suitable for use in human and veterinary medicine, where they are used, in particular, for the treatment and/or prophylaxis of disorders of the stomach and/or intestine

A further subject of the invention are therefore the compounds according to the invention for use in the treatment and/or prophylaxis of the abovementioned diseases.

The invention likewise includes the use of the compounds according to the invention for the production of medicaments which are employed for the treatment and/or prophylaxis of the abovementioned diseases.

The invention furthermore includes the use of the compounds according to the invention for the treatment and/or prophylaxis of the abovementioned diseases.

A further subject of the invention are medicaments which comprise one or more compounds of the formula 1 and/or their pharmacologically acceptable salts.

The medicaments are prepared by processes which are known per se and familiar to the person skilled in the art. As medicaments, the pharmacologically active compounds according to the invention (=active compounds) are either employed as such, or preferably in combination with suitable pharmaceutical auxiliaries or excipients in the form of tablets, coated tablets, capsules, suppositories, patches (e.g. as TTS), emulsions, suspensions or solutions, the active compound content advantageously being between 0.1 and 95% and it being possible to obtain a pharmaceutical administration form exactly adapted to the active compound and/or to the desired onset and/or duration of action (e.g. a sustained-release form or an enteric form) by means of the appropriate selection of the auxiliaries and excipients.

The auxiliaries and excipients which are suitable for the desired pharmaceutical formulations are known to the person skilled in the art on the basis of his/her expert knowledge. In addition to solvents, gel-forming agents, suppository bases, tablet auxiliaries and other active compound excipients, it is possible to use, for example, antioxidants, dispersants, emulsifiers, antifoams, flavor corrigents, preservatives, solubilizers, colorants or, in particular, permeation promoters and complexing agents (e.g. cyclodextrins).

The active compounds can be administered orally, parenterally or percutaneously.

In general, it has proven advantageous in human medicine to administer the active compound(s) in the case of oral administration in a daily dose of approximately 0.01 to approximately 20, preferably 0.05 to 5, in particular 0.1 to 1.5, mg/kg of body weight, if appropriate in the form of several, preferably 1 to 4, individual doses to achieve the desired result. In the case of a parenteral treatment, similar or (in particular in the case of the intravenous administration of the active compounds), as a rule, lower doses can be used. The establishment of the optimal dose and manner of administration of the active compounds necessary in each case can easily be carried out by any person skilled in the art on the basis of his/her expert knowledge.

If the compounds according to the invention and/or their salts are to be used for the treatment of the abovementioned diseases, the pharmaceutical preparations can also contain one or more pharmacologically active constituents of other groups of medicaments, for example: tranquilizers (for example from the group of the benzodiazepines, for example diazepam), spasmolytics (for example, bietamiverine or camylofine), anticholinergics (for example, oxyphencyclimine or phencarbamide), local anesthetics, (for example, tetracaine or procaine), and, if appropriate, also enzymes, vitamins or amino acids.

To be emphasized in this connection is in particular the combination of the compounds according to the invention with pharmaceuticals which inhibit acid secretion, such as, for example, H₂ blockers (e.g. cimetidine, ranitidine), H⁺/K⁺ ATPase inhibitors (e.g. omeprazole, pantoprazole), or further with so-called peripheral anticholinergics (e.g. pirenzepine, telenzepine) and with gastrin antagonists with the aim of increasing the principal action in an additive or super-additive sense and/or of eliminating or of decreasing the side effects, or further the combination with antibacterially active substances (such as, for example, cephalosporins, tetracyclines, penicillins, macrolides, nitroimidazoles or alternatively bismuth salts) for the control of Helicobacter pylori. Suitable antibacterial co-components which may be mentioned are, for example, mezlocillin, ampicillin, amoxicillin, cefalothin, cefoxitin, cefotaxime, imipenem, gentamycin, amikacin, erythromycin, ciprofloxacin, metronidazole, clarithromycin, azithromycin and combinations thereof (for example clarithromycin+metronidazole).

In view of their excellent gastric and intestinal protection action, the compounds of formula 1 are suited for a free or fixed combination with those medicaments (e.g. certain antiinflammatories and antirheumatics, such as NSAIDs), which are known to have a certain ulcerogenic potency. In addition, the compounds of formula 1 are suited for a free or fixed combination with motility-modifying drugs.

Pharmacology

The excellent gastric protective action and the gastric acid secretion-inhibiting action of the compounds according to the invention can be demonstrated in investigations on animal experimental models. The compounds of the formula 1 according to the invention investigated in the model mentioned below have been provided with numbers which correspond to the numbers of these compounds in the examples.

Testing of the Secretion-Inhibiting Action on the Perfused Rat Stomach

In Table A which follows, the influence of the compounds of the formula 1 according to the invention on the pentagastrin-stimulated acid secretion of the perfused rat stomach after intraduodenal administration in vivo is shown.

TABLE A

| | Dose | Inhibition of |
|---|---|---|
| | (μmol/kg) | acid secretion |
| No./letters | i.d. | (%) |
| 4 | 1 | >40 |
| 6 | 1 | >40 |
| 9 | 1 | >40 |
| 11 | 1 | >70 |
| 13 | 1 | >70 |

In Table B which follows, the influence of the compounds of the formula 1-a according to the invention and of their optical antipodes of the formula 1-b on the pentagastrin-stimulated acid secretion of the perfused rat stomach after intraduodenal administration in vivo is shown.

TABLE B

| | Dose | Inhibition of | | Dose | Inhibition of |
|---|---|---|---|---|---|
| | (μmol/kg) | acid secretion | | (μmol/kg) | acid secretion |
| No. | i.d. | (%) | Letters | i.d. | (%) |
| A | 1 | >50 | a | 3 | <40 |
| B | 1 | 100 | b | 3 | <50 |
| C | 1 | 100 | c | 3 | <50 |

Methodology

The abdomen of anesthetized rats (CD rat, female, 200-250 g; 1.5 g/kg i.m. urethane) was opened after tracheotomy by a median upper abdominal incision and a PVC catheter was fixed transorally in the esophagus and another via the pylorus such that the ends of the tubes just projected into the gastric lumen. The catheter leading from the pylorus led outward into the right abdominal wall through a side opening.

After thorough rinsing (about 50-100 ml), warm (37° C.) physiological NaCl solution was continuously passed through the stomach (0.5 ml/min, pH 6.8-6.9; Braun-Unita I). The pH (pH meter 632, glass electrode EA 147; φ=5 mm, Metrohm) and, by titration with a freshly prepared 0.01N NaOH solution to pH 7 (Dosimat 665 Metrohm), the secreted HCl were determined in the effluent in each case collected at an interval of 15 minutes.

The gastric secretion was stimulated by continuous infusion of 1 μg/kg (=1.65 ml/h) of i.v. pentagastrin (left femoral vein) about 30 min after the end of the operation (i.e. after determination of 2 preliminary fractions). The substances to be tested were administered intraduodenally in a 2.5 ml/kg liquid volume 60 min after the start of the continuous pentagastrin infusion. The body temperature of the animals was kept at a constant 37.8-38° C. by infrared irradiation and heat pads (automatic, stepless control by means of a rectal temperature sensor).

1. A compound of the formula 1 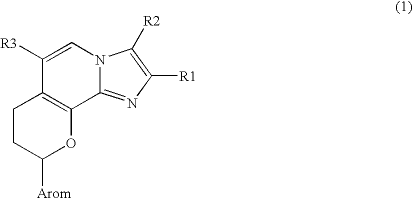
in which
R1 is hydrogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl or hydroxy-1-4C-alkyl,
R2 is hydrogen, 1-4C-alkyl, 3-7C-cycloalkyl, 3-7C-cycloalkyl-1-4C-alkyl, 1-4C-alkoxycarbonyl, hydroxy-1-4C-alkyl, hydroxy-3-4-C-alkenyl, hydroxy 3-4C-alkinyl, halogen, 2-4C-alkenyl, 2-4C-alkynyl, fluoro-1-4C-alkyl, cyanomethyl, hydroxy, 1-4C-alkoxy, amino, mono- or di-1-4C-alkylamino, 1-4C-alkylcarbonylamino, 1-4C-alkoxycarbonylamino, 1-4C-alkoxy-1-4C-alkoxycarbonylamino, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR21R22,
where
R21 is hydrogen, 1-7C-alkyl, hydroxy-1-4C-alkyl, 1-4C-alkoxy-1-4C-alkyl or 3-7C-cycloalkyl and
R22 is hydrogen, 1-7C-alkyl, hydroxy-1-4C-alkyl, 1-4C-alkoxy-1-4C-alkyl or 3-7C-cycloalkyl,
or where
R21 and R22 together and including the nitrogen atom to which they are attached form a pyrrolidino, piperidino, morpholino, aziridino or azetidino radical,
R3 is 1-4C-alkylcarbonyl, hydroxy-1-4C-alkyl, 1-4C-alkoxy-1-4C-alkyl, 1-4C-alkoxy-1-4C-alkoxy-1-4C-alkyl, 1-4C-alkoxycarbonyl, fluoro-1-4C-alkoxy-1-4C-alkyl, cyano, the radical —CO—NR31R32, the radical —SO₂—NR31R32, the radical —CS—NR31R32, the radical —C═N(OH)—NR31R32 or the group Het,
where
R31 is hydrogen, amino, 1-7C-alkyl, hydroxy, hydroxy-1-4C-alkyl, 1-4C-alkoxy, 1-4C-alkoxy-1-4C-alkyl, 3-7C-cycloalkyl, 1-4C-alkylsulfonyl, arylsulfonyl, aryl-1-4C-alkylsulfonyl or aryl, and
R32 is hydrogen, 1-7C-alkyl, hydroxy-1-4C-alkyl, 1-4C-alkoxy-1-4C-alkyl or 3-7C-cycloalkyl,
or where
R31 and R32 together and including the nitrogen atom to which they are attached form a cyclic residue, substituted by R33, R34 and R35, selected from the group consisting of pyrrolidino, piperidino, piperazino, morpholino, aziridino and azetidino, and
Het is a heterocyclic residue, substituted by R33, R34 and R35, selected from the group consisting of oxadiazol, dihydrooxazol, dihydroimidazol, oxazol, imidazol, isoxazol, dihydroisoxazol, pyrazol, and tetrazol,
where
R33 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R34 is hydrogen, 1-4C-alkyl, 1-4C-alkoxy, 1-4C-alkoxycarbonyl, halogen, trifluoromethyl or hydroxy,
R35 is hydrogen, 1-4C-alkyl, 1-4C-alkoxy, 1-4C-alkoxycarbonyl, halogen, trifluoromethyl or hydroxy,
Arom is a R4-, R5-, R6- and R7-substituted mono- or bicyclic aromatic radical selected from the group consisting of phenyl, naphthyl, pyrrolyl, pyrazolyl, imidazolyl, 1,2,3-triazolyl, indolyl, benzimidazolyl, furanyl (furyl), benzofuranyl (benzofuryl), thiophenyl (thienyl), benzothiophenyl (benzothienyl), thiazolyl, isoxazolyl, pyridinyl, pyrimidinyl, quinolinyl and isoquinolinyl,
where
R4 is hydrogen, 1-4C-alkyl, hydroxy-1-4C-alkyl, 1-4C-alkoxy, 2-4C-alkenyloxy, 1-4C-alkylcarbonyl, carboxyl, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryloxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R5 is hydrogen, 1-4C-alkyl, 1-4C-alkoxy, 1-4C-alkoxycarbonyl, halogen, trifluoromethyl or hydroxy,
R6 is hydrogen, 1-4C-alkyl or halogen and
R7 is hydrogen, 1-4C-alkyl or halogen,
where
aryl is phenyl or substituted phenyl having one, two or three identical or different substitutents selected from the group consisting of 1-4C-alkyl, 1-4C-alkoxy, carboxyl, 1-4C-alkoxycarbonyl, halogen, trifluoromethyl, nitro, trifluoromethoxy, hydroxy and cyano,
with the proviso that,
when
R2 is hydrogen, 1-4C-alkyl, 3-7C-cycloalkyl, 3-7C-cycloalkyl-1-4C-alkyl, 1-4C-alkoxycarbonyl, hydroxy-1-4C-alkyl, halogen, 2-4C-alkenyl, 2-4C-alkynyl, fluoro-1-4C-alkyl or cyanomethyl,
then
R3 is 1-4C-alkylcarbonyl, cyano, the radical —CO—NR31R32, the radical —SO₂—NR31R32, the radical —CS—NR31R32, the radical C═N(OH)—NR31R32 or the group Het,
where for the radical —CO—NR31R32,
R31 is amino, hydroxy, 1-4C-alkoxy, 3-7C-cycloalkyl, 1-4C-alkylsulfonyl, arylsulfonyl, aryl-1-4C-alkylsulfonyl or aryl and
R32 is hydrogen, 1-7C-alkyl, hydroxy-1-4C-alkyl, 1-4C-alkoxy-1-4C-alkyl or 3-7C-cycloalkyl,
and for the radicals —SO₂—NR31R32, —CS—NR31R32, and C═N(OH)—NR31R32,
R31 is hydrogen, amino, 1-7C-alkyl, hydroxy, hydroxy-1-4C-alkyl, 1-4C-alkoxy, 1-4C-alkoxy-1-4C-alkyl, 3-7C-cycloalkyl, 1-4C-alkylsulfonyl, arylsulfonyl, aryl-1-4C-alkylsulfonyl or aryl, and
R32 is hydrogen, 1-7C-alkyl, hydroxy-1-4C-alkyl, 1-4C-alkoxy-1-4C-alkyl or 3-7C-cycloalkyl,
or where
R31 and R32 together and including the nitrogen atom to which they are attached form a cyclic residue, substituted by R33, R34 and R35, selected from the group consisting of pyrrolidino, piperidino, piperazino, morpholino, aziridino and azetidino, where in the case of pyrrolidino, piperidino, or morpholino, at least one of the substitutents R33, R34, or R35 has to be different from hydrogen, and
Het is a heterocyclic residue, substituted by R33, R34 and R35, selected from the group consisting of oxadiazol, dihydrooxazol, dihydroimidazol, oxazol, imidazol, isoxazol, dihydroisoxazol, pyrazol, and tetrazol,
where
R33 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R34 is hydrogen, 1-4C-alkyl, 1-4C-alkoxy, 1-4C-alkoxycarbonyl, halogen, trifluoromethyl or hydroxy,
R35 is hydrogen, 1-4C-alkyl, 1-4C-alkoxy, 1-4C-alkoxycarbonyl, halogen, trifluoromethyl or hydroxy,
or a salt thereof.

2. A compound of the formula 1 as claimed in claim 1, in which
R1 is hydrogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl or hydroxy-1-4C-alkyl,
R2 is hydrogen, 1-4C-alkyl, 3-7C-cycloalkyl, 3-7C-cycloalkyl-1-4C-alkyl, 1-4C-alkoxycarbonyl, hydroxy-1-4C-alkyl, hydroxy-3-4C-alkenyl, hydroxy-3-4C-alkinyl, halogen, 2-4C-alkenyl, 2-4C-alkynyl, fluoro-1-4C-alkyl, cyanomethyl, hydroxy, 1-4C-alkoxy, amino, mono- or di-1-4C-alkylamino, 1-4C-alkylcarbonylamino, 1-4C-alkoxycarbonylamino, 1-4C-alkoxy-1-4C-alkoxycarbonylamino, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR21R22,
where
R21 is hydrogen, 1-7C-alkyl, hydroxy-1-4C-alkyl, 1-4C-alkoxy-1-4C-alkyl or 3-7C-cycloalkyl and
R22 is hydrogen, 1-7C-alkyl, hydroxy-1-4C-alkyl, 1-4C-alkoxy-1-4C-alkyl or 3-7C-cycloalkyl,
or where
R21 and R22 together and including the nitrogen atom to which they are attached form a pyrrolidino, piperidino, morpholino, aziridino or azetidino radical,
R3 is hydroxy-1-4C-alkyl, 1-4C-alkoxy-1-4C-alkyl, 1-4C-alkoxy-1-4C-alkoxy-1-4C-alkyl, 1-4C-alkoxycarbonyl, fluoro-1-4C-alkoxy-1-4C-alkyl, an imidazolyl, tetrazolyl or oxazolyl radical or the radical —CO—NR31R32,
where
R31 is hydrogen, 1-7C-alkyl, hydroxy-1-4C-alkyl, 1-4C-alkoxy-1-4C-alkyl or 3-7C-cycloalkyl, and
R32 is hydrogen, 1-7C-alkyl, hydroxy-1-4C-alkyl, 1-4C-alkoxy-1-4C-alkyl or 3-7C-cycloalkyl,
or where
R31 and R32 together and including the nitrogen atom to which they are attached form a pyrrolidino, piperidino, morpholino, aziridino or azetidino radical,
Arom is a R4-, R5-, R6- and R7-substituted mono- or bicyclic aromatic radical selected from the group consisting of phenyl, naphthyl, pyrrolyl, pyrazolyl, imidazolyl, 1,2,3-triazolyl, indolyl, benzimidazolyl, furanyl (furyl), benzofuranyl (benzofuryl), thiophenyl (thienyl), benzothiophenyl (benzothienyl), thiazolyl, isoxazolyl, pyridinyl, pyrimidinyl, quinolinyl and isoquinolinyl,
where
R4 is hydrogen, 1-4C-alkyl, hydroxy-1-4C-alkyl, 1-4C-alkoxy, 2-4C-alkenyloxy, 1-4C-alkylcarbonyl, carboxyl, 1-4C-alkoxycarbonyl, carboxy-1-4C-alkyl, 1-4C-alkoxycarbonyl-1-4C-alkyl, halogen, hydroxyl, aryl, aryl-1-4C-alkyl, aryloxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R5 is hydrogen, 1-4C-alkyl, 1-4C-alkoxy, 1-4C-alkoxycarbonyl, halogen, trifluoromethyl or hydroxyl,
R6 is hydrogen, 1-4C-alkyl or halogen and
R7 is hydrogen, 1-4C-alkyl or halogen,
where
aryl is phenyl or substituted phenyl having one, two or three identical or different substitutents selected from the group consisting of 1-4C-alkyl, 1-4C-alkoxy, carboxyl, 1-4C-alkoxycarbonyl, halogen, trifluoromethyl, nitro, trifluoromethoxy, hydroxyl and cyano,
with the proviso that,
when
R2 is hydrogen, 1-4C-alkyl, 3-7C-cycloalkyl, 3-7C-cycloalkyl-1-4C-alkyl, 1-4C-alkoxycarbonyl, hydroxy-1-4C-alkyl, halogen, 2-4C-alkenyl, 2-4C-alkynyl, fluoro-1-4C-alkyl or cyanomethyl,
then
R3 is an imidazolyl, tetrazolyl or oxazolyl radical or the radical —CO—NR31R32,
where
R31 is 3-7C-cycloalkyl and
R32 is hydrogen, 1-7C-alkyl, hydroxy-1-4C-alkyl, 1-4C-alkoxy-1-4C-alkyl or 3-7C-cycloalkyl,
or where R31 and R32 together and including the nitrogen atom to which they are attached form an aziridino or azetidino radical,
or a salt thereof.

3. A compound of the formula 1 as claimed in claim 1, in which
R1 is hydrogen, 1-4C-alkyl or 3-7C-cycloalkyl,
R2 is hydrogen, 1-4C-alkyl, 3-7C-cycloalkyl, 3-7C-cycloalkyl-1-4C-alkyl, 1-4C-alkoxycarbonyl, hydroxy-1-4C-alkyl, hydroxy-3-4C-alkenyl, hydroxy-3-4C-alkinyl, halogen, 2-4C-alkenyl, 2-4C-alkynyl, fluoro-1-4C-alkyl, cyanomethyl, hydroxy, 1-4C-alkoxy, amino, mono- or di-1-4C-alkylamino, 1-4C-alkylcarbonylamino, 1-4C-alkoxycarbonylamino, 1-4C-alkoxy-1-4C-alkoxycarbonylamino, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR21R22,
where
R21 is hydrogen, 1-7C-alkyl, hydroxy-1-4C-alkyl, 1-4C-alkoxy-1-4C-alkyl or 3-7C-cycloalkyl, and
R22 is hydrogen, 1-7C-alkyl, hydroxy-1-4C-alkyl, 1-4C-alkoxy-1-4C-alkyl or 3-7C-cycloalkyl,
or where
R21 and R22 together and including the nitrogen atom to which they are attached form a pyrrolidino, piperidino, morpholino, aziridino or azetidino radical,
R3 is 1-4C-alkylcarbonyl, hydroxy-1-4C-alkyl, 1-4C-alkoxy-1-4C-alkyl, 1-4C-alkoxy-1-4C-alkoxy-1-4C-alkyl, 1-4C-alkoxycarbonyl, fluoro-1-4C-alkoxy-1-4C-alkyl, cyano, the radical —CO—NR31R32, the radical —SO₂—NR31R32, the radical —CS—NR31R32, the radical C═N(OH)—NR31R32 or the group Het,
where
R31 is hydrogen, amino, 1-7C-alkyl, hydroxy, hydroxy-1-4C-alkyl, 1-4C-alkoxy, 1-4C-alkoxy-1-4C-alkyl or 3-7C-cycloalkyl, 1-4C-alkylsulfonyl, arylsulfonyl, aryl-1-4C-alkylsulfonyl or aryl and
R32 is hydrogen, 1-7C-alkyl, hydroxy-1-4C-alkyl, 1-4C-alkoxy-1-4C-alkyl or 3-7C-cycloalkyl,
or where
R31 and R32 together and including the nitrogen atom to which they are attached form a cyclic residue, substituted by R33, R34 and R35, selected from the group consisting of pyrrolidino, piperidino, piperazino, morpholino, aziridino and azetidino, and
Het is a heterocyclic residue, substituted by R33, R34 and R35, selected from the group consisting of oxadiazol, dihydrooxazol, dihydroimidazol, oxazol, imidazol, isoxazol, dihydroisoxazol, pyrazol, and tetrazol,
where
R33 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R34 is hydrogen, 1-4C-alkyl, 1-4C-alkoxy, 1-4C-alkoxycarbonyl, halogen, trifluoromethyl or hydroxy,
R35 is hydrogen, 1-4C-alkyl, 1-4C-alkoxy, 1-4C-alkoxycarbonyl, halogen, trifluoromethyl or hydroxy,
where
aryl is phenyl or substituted phenyl having one, two or three identical or different substitutents selected from the group consisting of 1-4C-alkyl, 1-4C-alkoxy, carboxyl, 1-4C-alkoxycarbonyl, halogen, trifluoromethyl, nitro, trifluoromethoxy, hydroxy and cyano,
Arom is a R4- and R5-substituted phenyl, pyrrolyl, furanyl (furyl) or thiophenyl (thienyl) radical,
where
R4 is hydrogen, 1-4C-alkyl, halogen, 1-4C-alkoxy or trifluoromethyl,
R5 is hydrogen, 1-4C-alkyl or halogen,
with the proviso that,
when
R2 is hydrogen, 1-4C-alkyl, 3-7C-cycloalkyl, 3-7C-cycloalkyl-1-4C-alkyl, 1-4C-alkoxycarbonyl, hydroxy-1-4C-alkyl, halogen, 2-4C-alkenyl, 2-4C-alkynyl, fluoro-1-4C-alkyl or cyanomethyl,
then
R3 is 1-4C-alkylcarbonyl, cyano, the radical —CO—NR31R32, the radical —SO₂—NR31R32, the radical —CS—NR31R32, the radical C═N(OH)—NR31R32 or the group Het, where for the radical —CO—NR31R32,
R31 is amino, hydroxy, 1-4C-alkoxy, 3-7C-cycloalkyl, 1-4C-alkylsulfonyl, arylsulfonyl, aryl-1-4C-alkylsulfonyl or aryl and
R32 is hydrogen, 1-7C-alkyl, hydroxy-1-4C-alkyl, 1-4C-alkoxy-1-4C-alkyl or 3-7C-cycloalkyl,
and for the radicals —SO₂—NR31R32, —CS—NR31R32, and C═N(OH)—NR31R32,
R31 is hydrogen, amino, 1-7C-alkyl, hydroxy, hydroxy-1-4C-alkyl, 1-4C-alkoxy, 1-4C-alkoxy-1-4C-alkyl, 3-7C-cycloalkyl, 1-4C-alkylsulfonyl, arylsulfonyl, aryl-1-4C-alkylsulfonyl or aryl, and
R32 is hydrogen, 1-7C-alkyl, hydroxy-1-4C-alkyl, 1-4C-alkoxy-1-4C-alkyl or 3-7C-cycloalkyl,
or where
R31 and R32 together and including the nitrogen atom to which they are attached form a cyclic residue, substituted by R33, R34 and R35, selected from the group consisting of pyrrolidino, piperidino, piperazino, morpholino, aziridino and azetidino, where in the case of pyrrolidino, piperidino, or morpholino, at least one of the substitutents R33, R34, or R35 has to be different from hydrogen, and Het is a heterocyclic residue, substituted by R33, R34 and R35, selected from the group consisting of oxadiazol, dihydrooxazol, dihydroimidazol, oxazol, imidazol, isoxazol, dihydroisoxazol, pyrazol, and tetrazol,
where
R33 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R34 is hydrogen, 1-4C-alkyl, 1-4C-alkoxy, 1-4C-alkoxycarbonyl, halogen, trifluoromethyl or hydroxy,
R35 is hydrogen, 1-4C-alkyl, 1-4C-alkoxy, 1-4C-alkoxycarbonyl, halogen, trifluoromethyl or hydroxy,
where
aryl is phenyl or substituted phenyl having one, two or three identical or different substitutents selected from the group consisting of 1-4C-alkyl, 1-4C-alkoxy, carboxyl, 1-4C-alkoxycarbonyl, halogen, trifluoromethyl, nitro, trifluoromethoxy, hydroxy and cyano,
or a salt thereof.

4. A compound of the formula 1 as claimed in claim 1, in which
R1 is hydrogen, 1-4C-alkyl or 3-7C-cycloalkyl,
R2 is hydrogen, 1-4C-alkyl, hydroxy-3-4C-alkenyl, hydroxy-3-4C-alkinyl, hydroxy, 1-4C-alkoxy, amino, mono- or di-1-4C-alkylamino, 1-4C-alkylcarbonylamino, 1-4C-alkoxycarbonylamino, 1-4C-alkoxy-1-4C-alkoxycarbonylamino, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR21R22,
where
R21 is hydrogen, 1-7C-alkyl, hydroxy-1-4C-alkyl, 1-4C-alkoxy-1-4C-alkyl or 3-7C-cycloalkyl and
R22 is hydrogen, 1-7C-alkyl, hydroxy-1-4C-alkyl, 1-4C-alkoxy-1-4C-alkyl or 3-7C-cycloalkyl,
or where
R21 and R22 together and including the nitrogen atom to which they are attached form a pyrrolidino, piperidino, morpholino, aziridino or azetidino radical,
R3 is 1-4C-alkylcarbonyl, hydroxy-1-4C-alkyl, 1-4C-alkoxy-1-4C-alkyl, 1-4C-alkoxy-1-4C-alkoxy-1-4C-alkyl, 1-4C-alkoxycarbonyl, fluoro-1-4C-alkoxy-1-4C-alkyl, cyano, the radical —CO—NR31R32, the radical —SO₂—NR31R32, the radical —CS—NR31R32, the radical C═N(OH)—NR31R32 or the group Het,
where
R31 is hydrogen, amino, 1-7C-alkyl, hydroxy, hydroxy-1-4C-alkyl, 1-4C-alkoxy, 1-4C-alkoxy-1-4C-alkyl, 3-7C-cycloalkyl, 1-4C-alkylsulfonyl, arylsulfonyl, aryl-1-4C-alkylsulfonyl or aryl, and
R32 is hydrogen, 1-7C-alkyl, hydroxy-1-4C-alkyl, 1-4C-alkoxy-1-4C-alkyl or 3-7C-cycloalkyl,
or where
R31 and R32 together and including the nitrogen atom to which they are attached form a cyclic residue, substituted by R33, R34 and R35, selected from the group consisting of pyrrolidino, piperidino, piperazino, morpholino, aziridino and azetidino, and
Het is a heterocyclic residue, substituted by R33, R34 and R35, selected from the group consisting of oxadiazol, dihydrooxazol, dihydroimidazol, oxazol, imidazol, isoxazol, dihydroisoxazol, pyrazol, and tetrazol,
where
R33 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R34 is hydrogen, 1-4C-alkyl, 1-4C-alkoxy, 1-4C-alkoxycarbonyl, halogen, trifluoromethyl or hydroxy,
R35 is hydrogen, 1-4C-alkyl, 1-4C-alkoxy, 1-4C-alkoxycarbonyl, halogen, trifluoromethyl or hydroxy,
where
aryl is phenyl or substituted phenyl having one, two or three identical or different substitutents selected from the group consisting of 1-4C-alkyl, 1-4C-alkoxy, carboxyl, 1-4C-alkoxycarbonyl, halogen, trifluoromethyl, nitro, trifluoromethoxy, hydroxy and cyano,
Arom is a R4- and R5-substituted phenyl, pyrrolyl, furanyl (furyl) or thiophenyl (thienyl) radical,
where
R4 is hydrogen, 1-4C-alkyl, halogen, 1-4C-alkoxy or trifluoromethyl,
R5 is hydrogen, 1-4C-alkyl or halogen
with the proviso that,
when
R2 is hydrogen or 1-4C-alkyl,
then
R3 is 1-4C-alkylcarbonyl, cyano, the radical —CO—NR31R32, the radical —SO₂—NR31R32, the radical —CS—NR31R32, the radical C═N(OH)—NR31R32 or the group Het,
where for the radical —CO—NR31R32,
R31 is amino, hydroxy, 1-4C-alkoxy, 3-7C-cycloalkyl, 1-4C-alkylsulfonyl, arylsulfonyl, aryl-1-4C-alkylsulfonyl or aryl and
R32 is hydrogen, 1-7C-alkyl, hydroxy-1-4C-alkyl, 1-4C-alkoxy-1-4C-alkyl or 3-7C-cycloalkyl,
and for the radicals —SO₂—NR31R32, —CS—NR31R32, and C═N(OH)—NR31R32,
R31 is hydrogen, amino, 1-7C-alkyl, hydroxy, hydroxy-1-4C-alkyl, 1-4C-alkoxy, 1-4C-alkoxy-1-4C-alkyl, 3-7C-cycloalkyl, 1-4C-alkylsulfonyl, arylsulfonyl, aryl-1-4C-alkylsulfonyl or aryl and
R32 is hydrogen, 1-7C-alkyl, hydroxy-1-4C-alkyl, 1-4C-alkoxy-1-4C-alkyl or 3-7C-cycloalkyl,
or where
R31 and R32 together and including the nitrogen atom to which they are attached form a cyclic residue, substituted by R33, R34 and R35, selected from the group consisting of pyrrolidino, piperidino, piperazino, morpholino, aziridino and azetidino, where in the case of pyrrolidino, piperidino, or morpholino, at least one of the substitutents R33, R34, or R35 has to be different from hydrogen, and
Het is a heterocyclic residue, substituted by R33, R34 and R35, selected from the group consisting of oxadiazol, dihydrooxazol, dihydroimidazol, oxazol, imidazol, isoxazol, dihydroisoxazol, pyrazol, and tetrazol,
where
R33 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R34 is hydrogen, 1-4C-alkyl, 1-4C-alkoxy, 1-4C-alkoxycarbonyl, halogen, trifluoromethyl or hydroxy,
R35 is hydrogen, 1-4C-alkyl, 1-4C-alkoxy, 1-4C-alkoxycarbonyl, halogen, trifluoromethyl or hydroxy,
where
aryl is phenyl or substituted phenyl having one, two or three identical or different substitutents selected from the group consisting of 1-4C-alkyl, 1-4C-alkoxy, carboxyl, 1-4C-alkoxycarbonyl, halogen, trifluoromethyl, nitro, trifluoromethoxy, hydroxy and cyano,
or a salt thereof.

5. A compound of the formula 1 as claimed in claim 1, in which
R1 is 1-4C-alkyl,
R2 is 1-4C-alkyl, hydroxy-3-4C-alkenyl, hydroxy-3-4C-alkinyl, hydroxy, 1-4C-alkoxy, amino, mono- or di-1-4C-alkylamino, 1-4C-alkylcarbonylamino, 1-4C-alkoxycarbonylamino, 1-4C-alkoxy-1-4C-alkoxycarbonylamino, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR21R22,
where
R21 is hydrogen, 1-4C-alkyl, hydroxy-1-4C-alkyl, 1-4C-alkoxy-1-4C-alkyl or 3-7C-cycloalkyl and
R22 is hydrogen, 1-4C-alkyl, hydroxy-1-4C-alkyl, 1-4C-alkoxy-1-4C-alkyl or 3-7C-cycloalkyl,
or where
R21 and R22 together and including the nitrogen atom to which they are attached form a pyrrolidino, piperidino, morpholino, aziridino or azetidino radical,
R3 is cyano, the radical —CO—NR31R32, the radical —CS—NR31R32, or the group Het,
where
R31 is hydrogen, 1-7C-alkyl, 1-4C-alkoxy, 3-7C-cycloalkyl, 1-4C-alkylsulfonyl, arylsulfonyl, aryl-1-4C-alkylsulfonyl or aryl and
R32 is hydrogen, 1-7C-alkyl, or 3-7C-cycloalkyl,
or where
R31 and R32 together and including the nitrogen atom to which they are attached form a cyclic residue, substituted by R33, selected from the group consisting of pyrrolidino, piperidino, piperazino, morpholino, aziridino and azetidino, and
Het is a heterocyclic residue, substituted by R33, selected from the group consisting of oxadiazol, dihydrooxazol, dihydroimidazol, oxazol, imidazol, isoxazol, dihydroisoxazol, pyrazol, and tetrazol,
where
R33 is hydrogen, 1-4C-alkyl, 1-4C-alkoxy, 1-4C-alkylcarbonyl, 1-4C-alkoxycarbonyl, halogen or hydroxy,
where
aryl is phenyl or substituted phenyl having one, two or three identical or different substitutents selected from the group consisting of 1-4C-alkyl, 1-4C-alkoxy, carboxyl, 1-4C-alkoxycarbonyl, halogen, trifluoromethyl, nitro, trifluoromethoxy, hydroxy and cyano,
Arom is a R4- and R5-substituted phenyl, pyrrolyl, furanyl (furyl) or thiophenyl (thienyl) radical,
where
R4 is hydrogen, 1-4C-alkyl, halogen, 1-4C-alkoxy or trifluoromethyl,
R5 is hydrogen, 1-4C-alkyl or halogen,
with the proviso that,
when
R2 is 1-4C-alkyl,
then
R3 is cyano, the radical —CO—NR31R32, the radical —CS—NR31R32, or the group Het,
where for the radical —CO—NR31R32,
R31 is 1-4C-alkoxy, 3-7C-cycloalkyl, 1-4C-alkylsulfonyl, arylsulfonyl, aryl-1-4C-alkylsulfonyl or aryl, and
R32 is hydrogen, 1-7C-alkyl, or 3-7C-cycloalkyl,
and for the radical —CS—NR31R32,
R31 is hydrogen, 1-7C-alkyl, 1-4C-alkoxy, 3-7C-cycloalkyl, 1-4C-alkylsulfonyl, arylsulfonyl, aryl-1-4C-alkylsulfonyl or aryl and
R32 is hydrogen, 1-7C-alkyl, or 3-7C-cycloalkyl,
or where
R31 and R32 together and including the nitrogen atom to which they are attached form a cyclic residue, substituted by R33, selected from the group consisting of pyrrolidino, piperidino, piperazino, morpholino, aziridino and azetidino, where in the case of pyrrolidino, piperidino, or morpholino, the substitutent R33 has to be different from hydrogen, and
Het is a heterocyclic residue, substituted by R33, selected from the group consisting of oxadiazol, dihydrooxazol, dihydroimidazol, oxazol, imidazol, isoxazol, dihydroisoxazol, pyrazol, and tetrazol,
where
R33 is hydrogen, 1-4C-alkyl, 1-4C-alkoxy, 1-4C-alkylcarbonyl, 1-4C-alkoxycarbonyl, halogen or hydroxy,
where
aryl is phenyl or substituted phenyl having one, two or three identical or different substitutents selected from the group consisting of 1-4C-alkyl, 1-4C-alkoxy, carboxyl, 1-4C-alkoxycarbonyl, halogen, trifluoromethyl, nitro, trifluoromethoxy, hydroxy and cyano,
or a salt thereof.

6. A compound of the formula 1 as claimed in claim 1, in which
R1 is 1-4C-alkyl,
R2 is 1-4C-alkyl, hydroxy-3-4C-alkenyl, hydroxy-3-4C-alkinyl, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR21R22,
where
R21 is hydrogen, 1-4C-alkyl or 1-4C-alkoxy-1-4C-alkyl and
R22 is hydrogen, 1-4C-alkyl or 1-4C-alkoxy-1-4C-alkyl,
R3 is cyano, the radical —CO—NR31R32, the radical —CS—NR31R32, or the group Het,
where
R31 is hydrogen, 1-7C-alkyl, 1-4C-alkoxy, 3-7C-cycloalkyl, 1-4C-alkylsulfonyl, arylsulfonyl, aryl-1-4C-alkylsulfonyl or aryl and
R32 is hydrogen, 1-7C-alkyl, or 3-7C-cycloalkyl,
or where
R31 and R32 together and including the nitrogen atom to which they are attached form a cyclic residue, substituted by R33, selected from the group consisting of pyrrolidino, piperazino, aziridino and azetidino, and
Het is a heterocyclic residue, substituted by R33, selected from the group consisting of dihydrooxazol, dihydroimidazol, oxazol, imidazol, isoxazol, dihydroisoxazol, and tetrazol,
where
R33 is hydrogen, 1-4C-alkyl, 1-4C-alkoxy, 1-4C-alkylcarbonyl, 1-4C-alkoxycarbonyl, halogen or hydroxy,
where
aryl is phenyl or substituted phenyl having one, two or three identical or different substitutents selected from the group consisting of 1-4C-alkyl, 1-4C-alkoxy, 1-4C-alkoxycarbonyl, halogen or hydroxy,
Arom is a R4-substituted phenyl, pyrrolyl, furanyl (furyl) or thiophenyl (thienyl) radical,
where
R4 is hydrogen or 1-4C-alkyl, halogen, 1-4C-alkoxy or trifluoromethyl,
with the proviso that,
when
R2 is 1-4C-alkyl,
then
R3 is cyano, the radical —CO—NR31R32, the radical —CS—NR31R32, or the group Het,
where for —CO—NR31R32,
R31 is 1-4C-alkoxy, 3-7C-cycloalkyl, 1-4C-alkylsulfonyl, arylsulfonyl, aryl-1-4C-alkylsulfonyl or aryl and
R32 is hydrogen, 1-7C-alkyl, or 3-7C-cycloalkyl, and for —CS—NR31R32,
R31 is hydrogen, 1-7C-alkyl, 1-4C-alkoxy, 3-7C-cycloalkyl, 1-4C-alkylsulfonyl, arylsulfonyl, aryl-1-4C-alkylsulfonyl or aryl and
R32 is hydrogen, 1-7C-alkyl, or 3-7C-cycloalkyl,
or where
R31 and R32 together and including the nitrogen atom to which they are attached form a cyclic residue, substituted by R33, selected from the group consisting of pyrrolidino, piperazino, aziridino and azetidino, where in the case of pyrrolidino, the substitutent R33 has to be different from hydrogen, and
Het is a heterocyclic residue, substituted by R33, selected from the group consisting of dihydrooxazol, dihydroimidazol, oxazol, imidazol, isoxazol, dihydroisoxazol, and tetrazol,
where
R33 is hydrogen, 1-4C-alkyl, 1-4C-alkoxy, 1-4C-alkylcarbonyl, 1-4C-alkoxycarbonyl, halogen or hydroxy,
where
aryl is phenyl or substituted phenyl having one, two or three identical or different substitutents selected from the group consisting of 1-4C-alkyl, 1-4C-alkoxy, 1-4C-alkoxycarbonyl, halogen and hydroxy,
or a salt thereof.

7. A compound of the formula 1 as claimed in claim 1, in which
R1 is 1-4C-alkyl,
R2 is 1-4C-alkyl, hydroxy-3-4C-alkinyl, carboxyl, mono- or di-1-4C-alkylamino-1-4C-alkyl, 1-4C-alkylcarbonyl, 2-4C-alkinylcarbonyl or the radical —CO—NR21R22,
where
R21 is 1-4C-alkyl or 1-4C-alkoxy-1-4C-alkyl, and
R22 is hydrogen or 1-4C-alkyl,
R3 is cyano, an oxazolyl radical, the radical —CO—NR31R32, or the radical —CS—NR31R32,
where
R31 is 1-4C-alkyl, 3-7C-cycloalkyl, 1-4C-alkylsulfonyl, aryl or 1-4C-alkoxy,
R32 is hydrogen or 1-4C-alkyl,
or where
R31 and R32 together and including the nitrogen atom to which they are attached form an aziridino, azetidino, hydroxyazetidino, or piperazino radical, where aryl is phenyl or phenyl substituted with 1-4C-alkoxy,
Arom is phenyl,
with the proviso that
when
R2 is 1-4C-alkyl,
then
R3 is cyano, an oxazolyl radical, the radical —CO—NR31R32, or the radical —CS—NR31R32,
where for —CO—NR31R32,
R31 is 3-7C-cycloalkyl, 1-4C-alkylsulfonyl, aryl or 1-4C-alkoxy,
R32 is hydrogen or 1-4C-alkyl,
and for —CS—NR31R32,
R31 is 1-4C-alkyl,
R32 is 1-4C-alkyl,
or where
R31 and R32 together and including the nitrogen atom to which they are attached form an aziridino, azetidino, hydroxyazetidino, or piperazino radical,
or a salt thereof.

8. A compound of the formula 1 as claimed in claim 1, in which
R1 is 1-4C-alkyl,
R2 is hydroxy-3-4C-alkinyl, carboxyl, mono- or di-1-4C-alkylamino-1-4C-alkyl, 1-4C-alkylcarbonyl, 2-4C-alkinylcarbonyl or the radical —CO—NR21R22,
where
R21 is 1-4C-alkyl or 1-4C-alkoxy-1-4C-alkyl, and
R22 is hydrogen or 1-4C-alkyl,
R3 is the radical —CO—NR31R32,
where
R31 is 1-4C-alkyl,
R32 is 1-4C-alkyl,
Arom is phenyl,
or a salt thereof.

9. A compound of the formula 1 as claimed in claim 1, in which
R1 is 1-4C-alkyl,
R2 is 1-4C-alkyl,
R3 is cyano, an oxazolyl radical, the radical —CO—NR31R32, or the radical —CS—NR31R32,
where for —CO—NR31R32,
R31 is 3-7C-cycloalkyl, 1-4C-alkylsulfonyl, aryl or 1-4C-alkoxy,
R32 is hydrogen or 1-4C-alkyl,
and for —CS—NR31R32,
R31 is 1-4C-alkyl,
R32 is 1-4C-alkyl,
or where
R31 and R32 together and including the nitrogen atom to which they are attached form an aziridino, azetidino, hydroxyazetidino, or piperazino radical, where aryl is phenyl or phenyl substituted with 1-4C-alkoxy,
Arom is phenyl,
or a salt thereof.

10. A compound of the formula 1 as claimed in claim 1, in which
R1 is 1-4C-alkyl,
R2 is 1-4C-alkyl, hydroxy-3-4C-alkinyl, carboxyl, mono- or di-1-4C-alkylamino-1-4C-alkyl, 1-4C-alkylcarbonyl, 2-4C-alkinylcarbonyl or the radical —CO—NR21R22,
where
R21 is hydrogen, 1-4C-alkyl or 1-4C-alkoxy-1-4C-alkyl, and
R22 is hydrogen, 1-4C-alkyl or 1-4C-alkoxy-1-4C-alkyl,
R3 is an oxazolyl radical or the radical —CO—NR31R32,
where
R31 is 1-4C-alkyl or 3-7C-cycloalkyl,
R32 is hydrogen or 1-4C-alkyl,
or where
R31 and R32 together and including the nitrogen atom to which they are attached form an aziridino or azetidino radical,
Arom is phenyl,
with the proviso that
when
R2 is 1-4C-alkyl,
then
R3 is an oxazolyl radical or the radical —CO—NR31R32,
where
R31 is 3-7C-cycloalkyl,
R32 is hydrogen,
or where
R31 and R32 together and including the nitrogen atom to which they are attached form an aziridino or azetidino radical,
or a salt thereof.

11. A compound of the formula 1 as claimed in claim 1, in which
R1 is 1-4C-alkyl,
R2 is hydroxy-3-4C-alkinyl, carboxyl, mono- or di-1-4C-alkylamino-1-4C-alkyl, 1-4C-alkylcarbonyl, 2-4C-alkinylcarbonyl or the radical —CO—NR21R22,
where
R21 is hydrogen, 1-4C-alkyl or 1-4C-alkoxy-1-4C-alkyl, and
R22 is hydrogen, 1-4C-alkyl or 1-4C-alkoxy-1-4C-alkyl,
R3 is the radical —CO—NR31R32,
where
R31 is 1-4C-alkyl,
R32 is 1-4C-alkyl,
Arom is phenyl,
or a salt thereof.

12. A compound of the formula 1 as claimed in claim 1, in which
R1 is 1-4C-alkyl,
R2 is 1-4C-alkyl,
R3 is an oxazolyl radical or the radical —CO—NR31R32,
where
R31 is 3-7C-cycloalkyl,
R32 is hydrogen,
or where
R31 and R32 together and including the nitrogen atom to which they are attached form an aziridino or azetidino radical,
Arom is phenyl,
or a salt thereof.

13. A compound of the formula 1 as claimed in claim 1, in which
R1 is 1-4C-alkyl,
R2 is carboxyl, mono- or di-1-4C-alkylamino-1-4C-alkyl or the radical —CO—NR21R22,
where
R21 is hydrogen, 1-4C-alkyl or 1-4C-alkoxy-1-4C-alkyl, and
R22 is hydrogen, 1-4C-alkyl or 1-4C-alkoxy-1-4C-alkyl,
R3 is the radical —CO—NR31R32,
where
R31 is 1-4C-alkyl and
R32 is 1-4C-alkyl,
Arom is phenyl,
or a salt thereof.

14. A compound of the formula 1 as claimed in claim 1, characterized by the formula 1-a 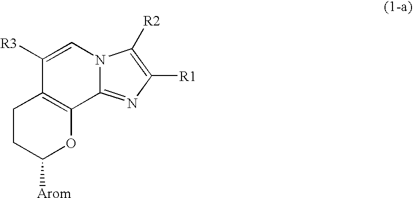
in which
R1 is hydrogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl or hydroxy-1-4C-alkyl,
R2 is hydrogen, 1-4C-alkyl, 3-7C-cycloalkyl, 3-7C-cycloalkyl-1-4C-alkyl, 1-4C-alkoxycarbonyl, hydroxy-1-4C-alkyl, hydroxy-3-4C-alkenyl, hydroxy-3-4C-alkinyl, halogen, 2-4C-alkenyl, 2-4C-alkynyl, fluoro-1-4C-alkyl, cyanomethyl, hydroxy, 1-4C-alkoxy, amino, mono- or di-1-4C-alkylamino, 1-4C-alkylcarbonylamino, 1-4C-alkoxycarbonylamino, 1-4C-alkoxy-1-4C-alkoxycarbonylamino, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR21R22,
where
R21 is hydrogen, 1-7C-alkyl, hydroxy-1-4C-alkyl, 1-4C-alkoxy-1-4C-alkyl or 3-7C-cycloalkyl and
R22 is hydrogen, 1-7C-alkyl, hydroxy-1-4C-alkyl, 1-4C-alkoxy-1-4C-alkyl or 3-7C-cycloalkyl,
or where
R21 and R22 together and including the nitrogen atom to which they are attached form a pyrrolidino, piperidino, morpholino, aziridino or azetidino radical,
R3 is 1-4C-alkylcarbonyl, hydroxy-1-4C-alkyl, 1-4C-alkoxy-1-4C-alkyl, 1-4C-alkoxy-1-4C-alkoxy-1-4C-alkyl, 1-4C-alkoxycarbonyl, fluoro-1-4C-alkoxy-1-4C-alkyl, cyano, the radical —CO—NR31R32, the radical —SO₂—NR31R32, the radical —CS—NR31R32, the radical —C═N(OH)—NR31R32 or the group Het,
where
R31 is hydrogen, amino, 1-7C-alkyl, hydroxy, hydroxy-1-4C-alkyl, 1-4C-alkoxy, 1-4C-alkoxy-1-4C-alkyl, 3-7C-cycloalkyl, 1-4C-alkylsulfonyl, arylsulfonyl, aryl-1-4C-alkylsulfonyl or aryl and
R32 is hydrogen, 1-7C-alkyl, hydroxy-1-4C-alkyl, 1-4C-alkoxy-1-4C-alkyl or 3-7C-cycloalkyl,
or where
R31 and R32 together and including the nitrogen atom to which they are attached form a cyclic residue, substituted by R33, R34 and R35, selected from the group consisting of pyrrolidino, piperidino, piperazino, morpholino, aziridino and azetidino, and
Het is a heterocyclic residue, substituted by R33, R34 and R35, selected from the group consisting of oxadiazol, dihydrooxazol, dihydroimidazol, oxazol, imidazol, isoxazol, dihydroisoxazol, pyrazol, and tetrazol,
where
R33 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R34 is hydrogen, 1-4C-alkyl, 1-4C-alkoxy, 1-4C-alkoxycarbonyl, halogen, trifluoromethyl or hydroxy,
R35 is hydrogen, 1-4C-alkyl, 1-4C-alkoxy, 1-4C-alkoxycarbonyl, halogen, trifluoromethyl or hydroxy,
Arom is a R4-, R5-, R6- and R7-substituted mono- or bicyclic aromatic radical selected from the group consisting of phenyl, naphthyl, pyrrolyl, pyrazolyl, imidazolyl, 1,2,3-triazolyl, indolyl, benzimidazolyl, furanyl (furyl), benzofuranyl (benzofuryl), thiophenyl (thienyl), benzothiophenyl (benzothienyl), thiazolyl, isoxazolyl, pyridinyl, pyrimidinyl, quinolinyl and isoquinolinyl,
where
R4 is hydrogen, 1-4C-alkyl, hydroxy-1-4C-alkyl, 1-4C-alkoxy, 2-4C-alkenyloxy, 1-4C-alkylcarbonyl, carboxyl, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryloxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R5 is hydrogen, 1-4C-alkyl, 1-4C-alkoxy, 1-4C-alkoxycarbonyl, halogen, trifluoromethyl or hydroxy,
R6 is hydrogen, 1-4C-alkyl or halogen and
R7 is hydrogen, 1-4C-alkyl or halogen,
where
aryl is phenyl or substituted phenyl having one, two or three identical or different substitutents selected from the group consisting of 1-4C-alkyl, 1-4C-alkoxy, carboxyl, 1-4C-alkoxycarbonyl, halogen, trifluoromethyl, nitro, trifluoromethoxy, hydroxy and cyano,
with the proviso that,
when
R2 is hydrogen, 1-4C-alkyl, 3-7C-cycloalkyl, 3-7C-cycloalkyl-1-4C-alkyl, 1-4C-alkoxycarbonyl, hydroxy-1-4C-alkyl, halogen, 2-4C-alkenyl, 2-4C-alkynyl, fluoro-1-4C-alkyl or cyanomethyl,
then
R3 is 1-4C-alkylcarbonyl, cyano, the radical —CO—NR31R32, the radical —SO₂—NR31R32, the radical —CS—NR31R32, the radical C═N(OH)—NR31R32 or the group Het,
where for the radical —CO—NR31R32,
R31 is amino, hydroxy, 1-4C-alkoxy, 3-7C-cycloalkyl, 1-4C-alkylsulfonyl, arylsulfonyl, aryl-1-4C-alkylsulfonyl or aryl and
R32 is hydrogen, 1-7C-alkyl, hydroxy-1-4C-alkyl, 1-4C-alkoxy-1-4C-alkyl or 3-7C-cycloalkyl,
and for the radicals —SO₂—NR31R32, —CS—NR31R32, and C═N(OH)—NR31R32,
R31 is hydrogen, amino, 1-7C-alkyl, hydroxy, hydroxy-1-4C-alkyl, 1-4C-alkoxy, 1-4C-alkoxy-1-4C-alkyl, 3-7C-cycloalkyl, 1-4C-alkylsulfonyl, arylsulfonyl, aryl-1-4C-alkylsulfonyl or aryl and
R32 is hydrogen, 1-7C-alkyl, hydroxy-1-4C-alkyl, 1-4C-alkoxy-1-4C-alkyl or 3-7C-cycloalkyl,
or where
R31 and R32 together and including the nitrogen atom to which they are attached form a cyclic residue, substituted by R33, R34 and R35, selected from the group consisting of pyrrolidino, piperidino, piperazino, morpholino, aziridino and azetidino, where in the case of pyrrolidino, piperidino, or morpholino, at least one of the substitutents R33, R34, or R35 has to be different from hydrogen, and
Het is a heterocyclic residue, substituted by R33, R34 and R35, selected from the group consisting of oxadiazol, dihydrooxazol, dihydroimidazol, oxazol, imidazol, isoxazol, dihydroisoxazol, pyrazol, and tetrazol,
where
R33 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R34 is hydrogen, 1-4C-alkyl, 1-4C-alkoxy, 1-4C-alkoxycarbonyl, halogen, trifluoromethyl or hydroxy,
R35 is hydrogen, 1-4C-alkyl, 1-4C-alkoxy, 1-4C-alkoxycarbonyl, halogen, trifluoromethyl or hydroxy,
or a salt thereof.

15. A compound of the formula 1 as claimed in claim 1, characterized by the formula 1-a 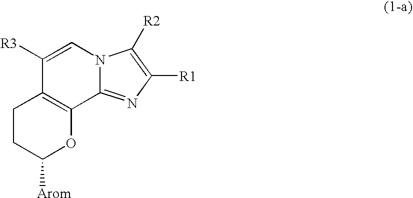
in which
R1 is 1-4C-alkyl,
R2 is 1-4C-alkyl or 1-4C-alkylcarbonyl,
R3 is the radical —CO—NR31R32 or the radical —CS—NR31R32,
where
R31 is 1-4C-alkyl or 3-7C-cycloalkyl,
R32 is hydrogen or 1-4C-alkyl,
or where
R31 and R32 together and including the nitrogen atom to which they are attached form an azetidino radical,
Arom is phenyl,
with the proviso that
when
R2 is 1-4C-alkyl,
then
R3 is the radical —CO—NR31R32 or the radical —CS—NR31R32,
where for —CO—NR31R32,
R31 is 3-7C-cycloalkyl,
R32 is hydrogen,
and for —CS—NR31R32,
R31 is 1-4C-alkyl,
R32 is 1-4C-alkyl,
or where
R31 and R32 together and including the nitrogen atom to which they are attached form an azetidino radical,
or a salt thereof.

16. A compound of the formula 1 as claimed in claim 1, characterized by the formula 1-a 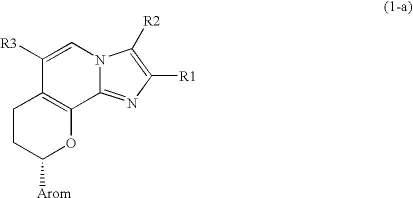
in which
R1 is 1-4C-alkyl,
R2 is 1-4C-alkyl,
R3 is the radical —CO—NR31R32 or the radical —CS—NR31R32,
where for —CO—NR31R32,
R31 is 3-7C-cycloalkyl,
R32 is hydrogen,
and for —CS—NR31R32,
R31 is 1-4C-alkyl,
R32 is 1-4C-alkyl,
or where
R31 and R32 together and including the nitrogen atom to which they are attached form an azetidino radical,
Arom is phenyl,
or a salt thereof.

17. A compound of the formula 1 as claimed in claim 1, characterized by the formula 1-a 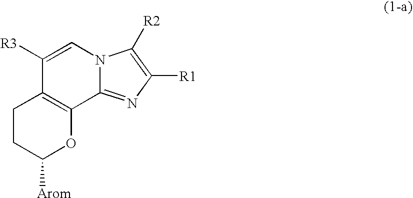
in which
R1 is 1-4C-alkyl,
R2 is 1-4C-alkylcarbonyl,
R3 is the radical —CO—NR31R32,
where
R31 is 1-4C-alkyl,
R32 is 1-4C-alkyl,
Arom is phenyl,
or a salt thereof.

18. The compound (9S)-2,3-Dimethyl-9-phenyl-7H-8,9-dihydro-pyrano[2,3-c]-imidazo[1,2-a]pyridine-6-carboxylic acid cyclopropylamide or a salt thereof.

19. The compound (9S)-(2,3-Dimethyl-9-phenyl-7H-8,9-dihydro-pyrano[2,3-c]-imidazo[1,2-a]pyridin-6-yl)-azetidin-1-yl methanone or a salt thereof.

20. A pharmaceutical composition comprising a compound as claimed in claim 1 and/or a pharmacologically acceptable salt thereof together with a pharmaceutically acceptable auxiliary and/or excipient.

21. (canceled)

22. A method of preventing or treating a gastrointestinal disorder in a patient comprising administering to a patient in need thereof a therapeutically effective amount of a compound as claimed in claim 1 or a pharmaceutically acceptable salt thereof.
